(19) United States
(12) Patent Application Publication
(10) Pub. No.: US 2014/0309168 A1
(43) Pub. Date: Oct. 16, 2014

(54) MODIFIED PEPTIDES AND PROTEINS

(71) Applicant: AmideBio, LLC, Boulder, CO (US)

(72) Inventor: Mary S. Rosendahl, Broomfield, CO (US)

(73) Assignee: AmideBio, LLC, Boulder, CO (US)

(21) Appl. No.: 14/219,792

(22) Filed: Mar. 19, 2014

Related U.S. Application Data
(63) Continuation of application No. 13/810,327, filed on Jan. 25, 2013, now abandoned.
(60) Provisional application No. 61/365,588, filed on Jul. 19, 2010.
(60) Provisional application No. 61/377,410, filed on Aug. 26, 2010.
(60) Provisional application No. 61/384,812, filed on Sep. 21, 2010.

(51) Int. Cl. C07K 14/605; C07K 14/00

(52) U.S. Cl. CPC C07K 14/605; C07K 14/001
USPC 514/11.7; 530/324; 530/308; 514/21.3

(57) ABSTRACT

Provided are compounds and methods of making compounds containing two or three groups derived from a peptide, such as enfuvirtide or exenatide, covalently bound to a linker. The compounds may contain polyethylene glycol groups to enhance solubility and pharmacokinetic properties. The compounds are useful for the treatment of diseases or conditions subject to treatment with the parent peptide, such as HIV and AIDS in the case of enfuvirtide, or diabetes in the case of exenatide.

This application claims priority from U.S. 61/365,588, filed Jul. 19, 2010, U.S. 61/377,410, filed Aug. 26, 2010, and U.S. 61/384,812, filed Sep. 21, 2010, the entire disclosures of which are herein incorporated by reference in their entirety.

### FIELD OF THE INVENTION

The invention describes compounds containing two or three groups derived from a peptide, such as enfuvirtide or exenatide, covalently bound to a linker. The compounds may contain polyethylene glycol groups to enhance solubility and pharmacokinetic properties. Compounds of the invention are useful for the treatment of diseases or conditions subject to treatment with the parent peptide, such as HIV and AIDS in the case of enfuvirtide, or diabetes in the case of exenatide. Compounds and methods of making and using the same are described.

### BACKGROUND

Naturally occurring peptides and proteins play an important role in modulating many physiological processes. Increasingly, proteins and peptides have proven to be useful for the treatment of disease.

One example of a therapeutic peptide is enfuvirtide, sold under the name Fuzeon®. Enfuvirtide is an FDA-approved antiviral fusion inhibitor, which prevents human immunodeficiency virus (HIV) from entering a cell. Enfuvirtide is believed to bind gp41, a viral fusion protein. Ordinarily, gp41 is complexed with gp120, but further complexation with CD4 is believed to expose gp41 to antagonism by enfuvirtide. Enfuvirtide administration can attenuate the symptoms or proliferation of HIV in a subject and improve the overall quality of life for patients with HIV or AIDS. However, a typical regimen requires subcutaneous injections twice daily of 90 mg of enfuvirtide.

Another example of a therapeutic peptide is exenatide, sold under the name Byetta®. Exenatide is an FDA-approved treatment for diabetes mellitus type 2, and is thought to be an insulin secretagogue with glucoregulatory effects. The peptide is a 39 amino acid synthetic version of exendin-4, a hormone found in the saliva of the Gila monster. Exenatide has a half-life of 2.4 hours. Thus, a 5 mcg dose of exenatide is typically administered as a subcutaneous injection to the abdomen, thigh, or arm, 30 to 60 minutes before the first and last meal of the day.

As seen with enfuvirtide and exenatide, one drawback of administering peptides as therapeutics is the limited half life of peptides in vivo. Additional drawbacks include limited bioavailability, undesired immunogenic responses, and limited efficacy.

Previous attempts to improve the properties of proteins and peptides have been made. For example, the covalent attachment of a polyethylene glycol (PEG) moiety to a protein or polypeptide (“PEGylation”) has been reported in U.S. Pat. No. 7,049,415, which discloses compounds comprising an enfuvirtide group and a single PEG group. The PEG-enfuvirtide complexes demonstrated IC₅₀ and IC₉₀ values in HIV inhibition assays. U.S. Pat. No. 7,049,415 is incorporated herein by reference in its entirety. However, such covalent attachment often leads to product heterogeneity due to attachment of the PEG moiety at random positions on the protein or peptide of interest.

Thus, there remains a continuing need for improving the properties of proteins and peptides.

### SUMMARY OF THE INVENTION

The present invention is directed to modified proteins and peptides with improved properties compared to unmodified versions of the proteins and peptides. Where the unmodified proteins and peptides have a therapeutic use, the modified versions may have properties leading to an improvement in the therapeutic use. A specific embodiment of the present invention is directed to compounds with improved properties compared to enfuvirtide. Such compounds may be useful for the treatment of HIV and AIDS in subjects. Another specific embodiment of the present invention is directed to compounds with improved properties compared to exenatide. Compounds of the invention based on exenatide may be useful for the treatment of diabetes mellitus type 2 in subjects diagnosed with diabetes or for the treatment of pre-diabetic individuals.

Compounds according to the invention include peptides modified by at least one covalent bond or an analog of the peptide, wherein said modified peptide or analog has an in vivo elimination half-life greater than the half-life of the unmodified peptide. Alternatively, the modified peptide or analog thereof has a higher binding affinity for its target than the binding affinity of the unmodified peptide for its target. In various embodiments, the modified peptide or analog thereof has a decreased affinity for non-therapeutic targets, thus resulting in greater specificity for the desired target apart from the actual affinity for the target, with potentially fewer adverse effects.

The modified peptide according to the invention differs from an unmodified peptide by the placement of a covalent bond. The difference between the peptide analog according to the invention and an unmodified peptide may be more extensive, including a difference in at least one modified or unmodified amino acid, at least one modified or unmodified non-natural amino acid, at least one amino acid analog, and combinations thereof. Such differences between the peptide analog and an unmodified peptide may result from addition, insertion, substitution, deletion, and combinations thereof. In one embodiment, the peptide analog has one additional amino acid, which may be a cysteine added at the amino terminus, added at the carboxy terminus, inserted between any two amino acids in the unmodified peptide, or inserted as a substitution for an amino acid in the unmodified peptide. In various embodiments, the peptide analog may have a sequence homology with the unmodified peptide of greater than about 75%, greater than about 80%, greater than about 85%, greater than about 90%, or greater than about 95%.

In some embodiments, compounds according to the invention include enfuvirtide modified by at least one covalent bond, or an enfuvirtide analog, wherein said modified enfuvirtide or analog has an in vivo elimination half-life of greater than about 3.8 hours and binds gp41 with about the same or greater affinity than enfuvirtide. In one embodiment, the compounds bind gp41 with a similar affinity as compared to enfuvirtide. Alternatively, compounds according to the invention include enfuvirtide modified by at least one covalent bond, or an enfuvirtide analog, wherein said modified enfuvirtide or analog has an in vivo elimination half-life of greater than about 3.8 hours and binds anti-thrombin with about the same or less affinity than enfuvirtide. In various embodiments, the enfuvirtide analog differs from enfuvirtide by at least one modified or unmodified amino acid, at least one modified or unmodified non-natural amino acid, at least one amino acid analog, or combinations thereof. For example, the enfuvirtide analog may have a sequence homology with enfuvirtide of greater than about 75%, greater than about 80%, greater than about 85%, greater than about 90%, or greater than 95%. The difference between enfuvirtide and the enfuvirtide analog may result from the addition, insertion, substitution, or deletion of one or more amino acids, including combinations of addition, insertion, substitution, and deletion of amino acids. In one embodiment, the enfuvirtide analog contains a cysteine, which may occur as an addition to the 36-amino acid sequence of enfuvirtide at the amino terminus, at the carboxy terminus, or as a non-terminal insertion between two amino acids, or which may occur as a substitution of any amino acid in the 36-amino acid chain of enfuvirtide. In one embodiment, the enfuvirtide analog has 37 amino acids.

In some embodiments compounds according to the invention include exenatide modified by at least one covalent bond or an exenatide analog, wherein said modified exenatide or analog has an in vivo elimination half-life of greater than about 2.4 hours and binds glucagon-like polypeptide-1 (GLP-1) receptor with about the same or greater affinity than exenatide. In one embodiment, compounds according to the invention bind GLP-1 receptor with a similar or greater affinity as compared to exenatide. In one embodiment, compounds according to the invention have a higher in vivo efficacy compared to exenatide because they have a longer half life and/or bivalent binding to the cell surface. In various embodiments, the exenatide analog differs from exenatide by at least one modified or unmodified amino acid, at least one modified or unmodified non-natural amino acid, at least one amino acid analog, or combinations thereof. For example, the exenatide analog may have a sequence homology with exenatide of greater than about 75%, greater than about 80%, greater than about 85%, greater than about 90%, or greater than 95%. The difference between exenatide and the exenatide analog may result from the addition, insertion, substitution, or deletion of one or more amino acids, including combinations of addition, insertion, substitution, and deletion of amino acids. In one embodiment, the exenatide analog contains a cysteine, which may occur as an addition to the 39-amino acid sequence of exenatide at the amino terminus, at the carboxy terminus, or as a non-terminal insertion between two amino acids, or which may occur as a substitution of any amino acid in the 39-amino acid chain of exenatide. In one embodiment, the exenatide analog has 40 amino acids.

Preferably, the compounds according to the invention have a longer period of physiological efficacy than unmodified peptide. For example, the compound may have an in vivo elimination half-life of greater than about 2.4 hours, greater than about 3.8 hours, greater than about 6 hours, greater than about 12 hours, or greater than about 18 hours, or more. In some embodiments, for example, the modified peptide is enfuvirtide and the compound may have an in vivo elimination half-life of greater than about 3.8 hours, greater than about 6 hours, greater than about 12 hours, or greater than about 18 hours, or more. In other embodiments, for example, the modified peptide is exenatide and the compound may have an in vivo elimination half-life of greater than about 2.4 hours, greater than about 6 hours, greater than about 12 hours, or greater than about 18 hours, or more.

In various embodiments, compounds according to the invention may contain two or three groups derived from a peptide, joined by a linker. For example, compounds according to the invention may be represented by the formula:

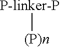

wherein P is a peptide modified to include a covalent bond to said linker, or P is a peptide analog, and n is 0 or 1. In various embodiments, the peptide analog has at least about 50% of the bioactivity of the non-modified peptide. For example, in certain embodiments, the peptide analog differs from the parent peptide in that the peptide analog contains at least one cysteine more than the parent peptide, or at least one modified natural amino acid, at least one modified or unmodified non-natural amino acid, at least one amino acid analog, or combinations thereof. Where n=0, compounds according to the invention may be represented by the formula:

P-linker-P.

In various embodiments, compounds according to the invention may contain two or three groups derived from enfuvirtide, joined by a linker. For example, compounds according to the invention may be represented by the formula:

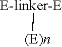

wherein E is enfuvirtide modified to include a covalent bond to said linker, or E is an enfuvirtide analog, and n is 0 or 1. In various embodiments, the enfuvirtide analog has at least about 50% of the bioactivity of enfuvirtide. For example, in certain embodiments, the enfuvirtide analog contains at least one cysteine, at least one modified natural amino acid, at least one modified or unmodified non-natural amino acid, at least one amino acid analog, or combinations thereof. Where n=0, compounds according to the invention may be represented by the formula:

E-linker-E.

In various embodiments, compounds according to the invention may contain two or three groups derived from exenatide, joined by a linker. For example, compounds according to the invention may be represented by the formula:

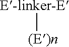

wherein E′ is exenatide modified to include a covalent bond to said linker, or E′ is an exenatide analog, and n is 0 or 1. In various embodiments, the exenatide analog has at least about 50% of the bioactivity of exenatide. For example, in certain embodiments, the exenatide analog contains at least one cysteine more than exenatide, at least one modified natural amino acid, at least one modified or unmodified non-natural amino acid, at least one amino acid analog, or combinations thereof. Where n=0, compounds according to the invention may be represented by the formula:

E′-linker-E′.

The linker, in addition be providing a covalent connection between the two or three appended groups, may also provide functional characteristics. For example, the linker may contain groups to enhance solubility and pharmacokinetic properties. In various embodiments, the linker contains a group selected from polyethylene glycol, polypropylene glycol, polyamine, polyamide, polyurethane, polyester and combinations thereof.

In some embodiments, the invention provides a compound of the formula (I):

Pep-S-L-(CH₂CH₂O)ₙ—CH₂CH₂-L-S-Pep,

wherein
each Pep is independently a peptide of the sequence:
X¹-(X)^{m}-X^{m+1}, wherein:
X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy;
each (X) independently represents an amino acid or G;
m is the number of independent (X) groups ranging from 0-1000;
G is a sulfur-containing moiety selected from Cys and a non-natural amino acid, such that each Pep includes at least one G;
and X^{m+1} is —N(R^{A})₂, -G-N(R^{A})₂, —O(R^{O}), or -G-O(R^{O}) wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl;
   each S is independently the sulfur atom of a G residue;
   each L is a linker group; and
   and n is an integer from 0-1,000;
   or a pharmaceutically-acceptable salt thereof.

In various embodiments, each linker group L independently comprises an electron-withdrawing group and a hydrocarbyl group, and optionally further comprises one or more of a polyethylene glycol group, an ester linkage, an amide linkage, a carbamate linkage, an ether linkage, and an amine linkage.

In some embodiments, the invention provides a compound of the formula (I):

Pep-S-L-(CH₂CH₂O)ₙ—CH₂CH₂-L-S-Pep,

wherein
each Pep is independently a peptide of the sequence:
X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy: X² is Tyr or G; X³ is Thr or G; X⁴ is Ser or G; X⁵ is Leu or G; X⁶ is Ile or G; X⁷ is His or G; X⁸ is Ser or G; X⁹ is Leu or G; X¹⁰ is Ile or G; X¹¹ is Glu or G; X¹² is Glu or G; X¹³ is Ser or G; X¹⁴ is Gln or G; X¹⁵ is Asn or G; X¹⁶ is Gln or G; X¹⁷ is Gln or G; X¹⁸ is Glu or G; X¹⁹ is Lys or G; X²⁰ is Asn or G; X²¹ is Glu or G; X²² is Gln or G; X²³ is Glu or G; X²⁴ is Leu or G; X²⁵ is Leu or G; X²⁶ is Glu or G; X²⁷ is Leu or G; X²⁸ is Asp or G; X²⁹ is Lys or G; X³⁰ is Trp or G; X³¹ is Ala or G; X³² is Ser or G; X³³ is Leu or G; X³⁴ is Trp or G; X³⁵ is Asn or G; X³⁶ is Trp or G; X³⁷ is Phe or G; and X³⁸ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently the sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and n is an integer from 0-1,000, or a pharmaceutically-acceptable salt thereof.

In some embodiments, the invention provides a compound of formula (I):

Pep-S-L-(CH₂CH₂O)ₙ—CH₂CH₂-L-S-Pep,

wherein
each Pep is independently a peptide of the sequence:
X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸-X³⁹-X⁴⁰-X⁴¹, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy; X² is His or G; X³ is Gly or G; X⁴ is Glu or G; X⁵ is Gly or G; X⁶ is Thr or G; X⁷ is Phe or G; X⁸ is Thr or G, X⁹ is Ser or G; X¹⁰ is Asp or G; X¹¹ is Leu or G; X¹² is Ser or G; X¹³ is Lys or G; X¹⁴ is Gln or G; X¹⁵ is Met or G; X¹⁶ is Glu or G; X¹⁷ is Glu or G; X¹⁸ is Glu or G; X¹⁹ is Ala or G; X²⁰ is Val or G; X²¹ is Arg or G; X²² is Leu or G; X²³ is Phe or G; X²⁴ is Ile or G; X²⁵ is Glu or G; X²⁶ is Trp or G; X²⁷ is Leu or G; X²⁸ is Lys or G; X²⁹ is Asn or G; X³⁰ is Gly or G; X³¹ is Gly or G; X³² is Pro or G; X³³ is Ser or G; X³⁴ is Ser or G; X³⁵ is Gly or G; X³⁶ is Ala or G; X³⁷ is Pro or G; X³⁸ is Pro or G; X³⁹ is Pro or G; X⁴⁰ is Ser or G; and X⁴¹ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently the sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and n is an integer from 0-1.000, or a pharmaceutically-acceptable salt thereof.

In some embodiments, the invention provides a pharmaceutical composition comprising (i) a compound of the formula as described herein, or a pharmaceutically-acceptable salt thereof, and (ii) one or more pharmaceutically-acceptable excipients.

The invention also provides a method of making a compound of the formula (I) as described herein, the method comprising contacting a compound of the formula (II):

RG-(CH₂CH₂O)ₙ—CH₂CH₂-RG

with a compound of the formula (III):

Pep-S—H

wherein each Pep, n, and S are independently as described herein, and further wherein RG is a reactive group, wherein each reactive group independently comprises a multiple bond in conjugation with an electron-withdrawing group, and optionally further comprises one or more of a hydrocarbyl group, a polyethylene glycol group, an ester linkage, an amide linkage, a carbamate linkage, an ether linkage, and an amine linkage. In various embodiments, the Pep group is enfuvirtide or an enfuvirtide analog. Alternatively, the Pep group is exenatide or an exenatide analog.

In some embodiments, the invention provides a method of treating HIV and/or AIDS in a subject, the method comprising administering to the subject a pharmaceutical composition comprising a therapeutically-effective amount of a compound according to formula (I), wherein the Pep group is enfuvirtide or an enfuvirtide analog.

In some embodiments, the invention provides a method of treating diabetes mellitus type 2 in a subject, the method comprising administering to the subject a pharmaceutical composition comprising a therapeutically-effective amount of a compound according to formula (I), wherein the Pep group is exenatide or an exenatide analog.

In alternate embodiments, the invention provides a compound of the formula (IV):

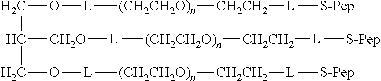

wherein
each Pep is independently a peptide of the sequence:
X¹-(X)^{m}-X^{m+1}, wherein:
X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy;
each (X) independently represents an amino acid or G;
m is the number of independent (X) groups ranging from 0-1000;
G is a sulfur-containing moiety selected from Cys and a non-natural amino acid, such that each Pep includes at least one G;
and X^{m+1} is —N(R^{A})₂, -G-N(R^{A})₂, —O(R^{O}), or -G-O(R^{O}) wherein R^{A} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl; each S is independently the sulfur atom of a G residue;
   each L is a linker group; and
   and n is an integer from 0-1,000;
   or a pharmaceutically-acceptable salt thereof.

In various embodiments, each linker group L independently comprises an electron-withdrawing group and a hydrocarbyl group, and optionally further comprises one or more of a polyethylene glycol group, an ester linkage, an amide linkage, a carbamate linkage, an ether linkage, and an amine linkage.

In alternate embodiments, the invention provides a compound of the formula (IV):

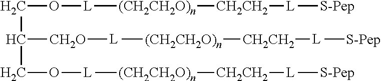

wherein
each Pep is independently a peptide of the sequence:

X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy: X² is Tyr or G; X³ is Thr or G; X⁴ is Ser or G; X⁵ is Leu or G; X⁶ is Ile or G; X⁷ is His or G; X⁸ is Ser or G; X⁹ is Leu or G; X¹⁰ is Ile or G; X¹¹ is Glu or G; X¹² is Glu or G; X¹³ is Ser or G; X¹⁴ is Gln or G; X¹⁵ is Asn or G; X¹⁶ is Gln or G; X¹⁷ is Gln or G; X¹⁸ is Glu or G; X¹⁹ is Lys or G; X²⁰ is Asn or G; X²¹ is Glu or G; X²² is Gln or G; X²³ is Glu or G; X²⁴ is Leu or G; X²⁵ is Leu or G; X²⁶ is Glu or G; X²⁷ is Leu or G; X²⁸ is Asp or G; X²⁹ is Lys or G; X³⁰ is Trp or G; X³¹ is Ala or G; X³² is Ser or G; X³³ is Leu or G; X³⁴ is Trp or G; X³⁵ is Asn or G; X³⁶ is Trp or G; X³⁷ is Phe or G; and X³⁸ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently the sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and n is an integer from 0-1,000, or a pharmaceutically-acceptable salt thereof.

In alternate embodiments, the invention provides a compound of the formula (IV):

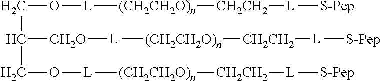

wherein
each Pep is independently a peptide of the sequence:
X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸-X³⁹-X⁴⁰-X⁴¹, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy; X² is His or G; X³ is Gly or G; X⁴ is Glu or G; X⁵ is Gly or G; X⁶ is Thr or G; X⁷ is Phe or G; X⁸ is Thr or G, X⁹ is Ser or G; X¹⁰ is Asp or G; X¹¹ is Leu or G; X¹² is Ser or G; X¹³ is Lys or G; X¹⁴ is Gln or G; X¹⁵ is Met or G; X¹⁶ is Glu or G; X¹⁷ is Glu or G; X¹⁸ is Glu or G; X¹⁹ is Ala or G; X²⁰ is Val or G; X²¹ is Arg or G; X²² is Leu or G; X²³ is Phe or G; X²⁴ is Ile or G; X²⁵ is Glu or G; X²⁶ is Trp or G; X²⁷ is Leu or G; X²⁸ is Lys or G; X²⁹ is Asn or G; X³⁰ is Gly or G; X³¹ is Gly or G; X³² is Pro or G; X³³ is Ser or G; X³⁴ is Ser or G; X³⁵ is Gly or G; X³⁶ is Ala or G; X³⁷ is Pro or G; X³⁸ is Pro or G; X³⁹ is Pro or G; X⁴⁰ is Ser or G; and X⁴¹ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently a sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and each n is independently an integer from 0-1,000, or a pharmaceutically-acceptable salt thereof.

In some embodiments, the invention provides a pharmaceutical composition comprising (i) a compound of the formula IV, or a pharmaceutically-acceptable salt thereof, and (ii) one or more pharmaceutically-acceptable excipients.

In some embodiments, the invention provides a method of making a compound of the formula (IV) comprising contacting a compound of the formula (VI):

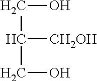

with a compound of the formula (II):

RG-(CH₂CH₂O)ₙ—CH₂CH₂-RG

thereby providing a compound of the formula (VII):

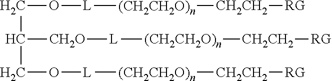

wherein each n and L are independently as described herein, and further wherein RG is a reactive group, wherein each reactive group independently comprises a multiple bond in conjugation with an electron-withdrawing group, and optionally further comprises one or more of a hydrocarbyl group, a polyethylene glycol group, an ester linkage, an amide linkage, a carbamate linkage, an ether linkage, and an amine linkage. The method further comprises contacting a compound of formula (VII) with a compound of the formula (III):

Pep-S—H

thereby providing a compound of the formula (IV), wherein Pep is as described herein. In various embodiments, the Pep group is enfuvirtide or an enfuvirtide analog. Alternatively, the Pep group is exenatide or an exenatide analog.

In some embodiments, the invention provides a method of making a compound of the formula (IV) comprising contacting a compound of the formula (II):

RG-(CH₂CH₂O)ₙ—CH₂CH₂-RG

with a compound of the formula (III):

Pep-S—H

thereby providing a compound of the formula (VIII):

RG-(CH₂CH₂O)ₙ—CH₂CH₂-L-S-Pep

wherein each n, L, S, and Pep are independently as described herein, and further wherein RG is a reactive group, wherein each reactive group independently comprises a multiple bond in conjugation with an electron-withdrawing group, and optionally further comprises one or more of a hydrocarbyl group, a polyethylene glycol group, an ester linkage, an amide linkage, a carbamate linkage, an ether linkage, and an amine linkage. The method further comprises contacting a compound of formula (VIII) with a compound of formula (VI):

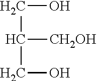

thereby providing a compound of formula (IV). In various embodiments, the Pep group is enfuvirtide or an enfuvirtide analog. Alternatively, the Pep group is exenatide or an exenatide analog.

In some embodiments, the invention provides a method of treating HIV and/or AIDS in a subject, the method comprising administering to the subject a pharmaceutical composition comprising a therapeutically-effective amount of a compound according to formula (IV), wherein the Pep group is enfuvirtide or an enfuvirtide analog.

In some embodiments, the invention provides a method of treating diabetes mellitus type 2 in a subject, the method comprising administering to the subject a pharmaceutical composition comprising a therapeutically-effective amount of a compound according to formula (IV), wherein the Pep group is exenatide or an exenatide analog.

In addition, the invention is directed to a vector comprising at least one promoter and sequences encoding each of the following: a) an affinity tag, b) an inclusion body targeting tag, c) a chemically cleavable tag, and d) a peptide. The affinity tag may be poly-histidine, poly-lysine, poly-aspartic acid, or poly-glutamic acid. The inclusion body targeting tag may be a ketoisomerase protein or fragment thereof. In various embodiments, the chemically cleavable tag is Trp, His-Met, or Pro-Met. In certain embodiments, the peptide is SEQ ID NO: 1 (enfuvirtide) or an enfuvirtide analog. In one embodiment, the enfuvirtide analog has at least 70% of the bioactivity of enfuvirtide and/or at least 80% sequence homology with enfuvirtide. Moreover, the enfuvirtide analog may contain at least one additional amino acid such as cysteine, at least one substitution of an amino acid for an amino acid in SEQ ID NO: 1, at least one modified natural amino acid, at least one modified or unmodified non-natural amino acid, at least one amino acid analog, or combinations thereof. In some embodiments, the peptide is SEQ ID NO: 78 (exenatide) or an exenatide analog. In one embodiment, the exenatide analog has at least 70% of the bioactivity of exenatide and/or at least 80% sequence homology with exenatide. Moreover, the exenatide analog may contain at least one additional amino acid such as cysteine, at least one substitution of an amino acid for an amino acid in SEQ ID NO: 78, at least one modified natural amino acid, at least one modified or unmodified non-natural amino acid, at least one amino acid analog, or combinations thereof.

In certain embodiments, the invention is directed to a method of producing a peptide or a peptide analog, said method comprising: i) obtaining a vector as described above; ii) transforming said vector into a host cell; iii) incubating the host cell for a time sufficient for production of peptides from said vector; iv) isolating the peptide or peptide analog from said incubating step. In one embodiment, the peptide analog has at least 70% of the bioactivity of the unmodified peptide. The method may also comprise the steps of v) separating inclusion bodies from the host cell; vi) extracting said inclusion bodies; vii) adding the extract to an affinity material; viii) washing the affinity material; ix) adding a chemical cleavage agent to the affinity material; x) separating cleaved product from the affinity material; and xi) optionally performing chemical modification of the amino and/or carboxy terminus and/or one or more amino acid side chains of the cleaved product.

In certain embodiments, the invention is directed to a method of producing enfuvirtide or an enfuvirtide analog, said method comprising: i) obtaining a vector as described above; ii) transforming said vector into a host cell; iii) incubating the host cell for a time sufficient for production of peptides from said vector; iv) isolating enfuvirtide or an enfuvirtide analog from said incubating step. In one embodiment, the enfuvirtide analog has at least 70% of the bioactivity of enfuvirtide. The method may also comprise the steps of v) separating inclusion bodies from the host cell; vi) extracting said inclusion bodies; vii) adding the extract to an affinity material; viii) washing the affinity material; ix) adding a chemical cleavage agent to the affinity material; x) separating cleaved product from the affinity material; and xi) optionally performing chemical modification of the amino and/or carboxy terminus and/or one or more amino acid side chains of the cleaved product.

In certain embodiments, the invention is directed to a method of producing exenatide or an exenatide analog, said method comprising: i) obtaining a vector as described above; ii) transforming said vector into a host cell; iii) incubating the host cell for a time sufficient for production of peptides from said vector; iv) isolating exenatide or an exenatide analog from said incubating step. In one embodiment, the exenatide analog has at least 70% of the bioactivity of exenatide. The method may also comprise the steps of v) separating inclusion bodies from the host cell; vi) extracting said inclusion bodies; vii) adding the extract to an affinity material; viii) washing the affinity material; ix) adding a chemical cleavage agent to the affinity material; x) separating cleaved product from the affinity material; and xi) optionally performing chemical modification of the amino and/or carboxy terminus and/or one or more amino acid side chains of the cleaved product.

### BRIEF DESCRIPTION OF THE DRAWINGS

FIG. 1 illustrates general formulas (I) and (IV) of compounds of the invention.

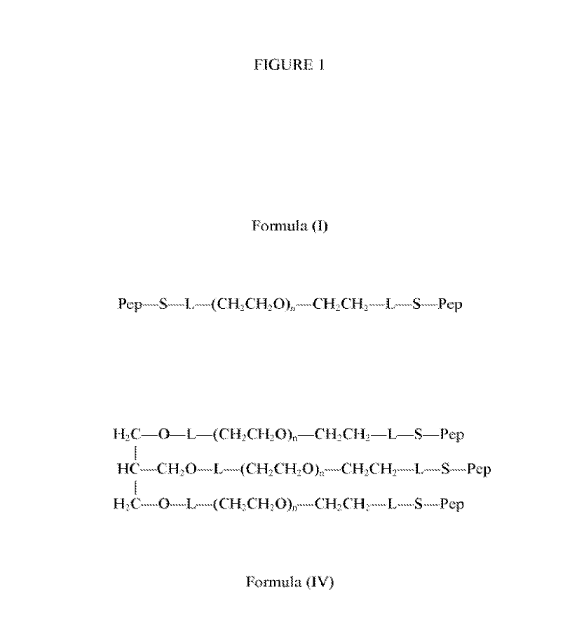

TABLES 1 and 2 provide SEQ ID NOs: 1-160.

### DETAILED DESCRIPTION OF THE INVENTION

The therapeutic utility of some proteins and peptides is limited by a short in vivo half life which can require a high frequency of dosing. For example, the therapeutic utility of enfuvirtide is limited by an in vivo half life of about 3.8 hours, while the therapeutic utility of exenatide is limited by an in vivo half life of about 2.4 hours. The invention described herein provides compounds that are characterized by desirable in vivo properties, pharmaceutical compositions comprising the same, methods of making the same, and methods of providing therapy to a subject.

The compounds according to the invention may include two or three peptides or peptide analogs in a covalently-bound complex. Thus, the desirable in vivo properties of the compounds of the invention may be understood by comparison to unmodified peptides. Without wishing to be bound by theory, the availability of multiple peptides in a single molecule may enhance the affinity of the compounds for their molecular target owing to the ability to bind multiple receptors concomitantly. This ability may provide compounds with improved efficacy and/or binding affinity. Alternatively, the compounds may have improved pharmacokinetic properties.

### DEFINITIONS

Unless defined otherwise, all technical and scientific terms used herein have the same meaning as commonly understood by a person of ordinary skill in the art.

As used herein, the term “peptide” is intended to mean any polymer of amino acids linked by peptide bonds. The term “peptide” is intended to include polymers that are assembled by enzymes as well as polymers assembled using a ribosome. In one embodiment, the peptide is produced synthetically. The term “peptide” may be considered synonymous with “protein,” or in various embodiments, the term “peptide” may be limited to a polymer of 50 or fewer amino acids wherein the polymer is produced synthetically or recombinantly.

As used herein, “consisting essentially of” may exclude those features not listed herein that would otherwise alter the operation of the invention. However, the use of the phrase “consisting essentially of” does not exclude features that do not alter the operation of the required components.

The term “polymer” is a molecule (or macromolecule) composed of repeating structural units connected by covalent chemical bonds.

A “patient,” “subject” or “host” to be treated with the composition of the present invention may mean either a human or non-human animal. The term “mammal” is known in the art, and exemplary mammals include humans, primates, bovines, porcines, canines, felines, and rodents (e.g. mice and rats).

Compounds

Compounds according to the invention may be modified forms of peptides, such as a fusion compound linking a peptide to one or more other moieties through a covalent bond. In another aspect, compounds according to the invention may be peptide analogs. For example, one or more amino acids from a peptide made be selectively altered so that the peptide analog has an amino acid sequence with at least one amino acid that is different from the sequence of the unmodified peptide, such as the inclusion or addition of cysteine, or at least one modified natural amino acid, or at least one modified or unmodified non-natural amino acid, or at least one amino acid analog, or combinations thereof.

Compounds according to the invention may be modified forms of enfuvirtide, such as a fusion compound linking enfuvirtide to one or more other moieties through a covalent bond. Alternatively, compounds according to the invention may be modified forms of exenatide, such as a fusion compound linking exenatide to one or more other moieties through a covalent bond. In another aspect, compounds according to the invention may be exenatide analogs.

In some embodiments, the compounds of the invention include a polyethylene glycol (PEG) group. The PEG group acts as a linker between peptide groups and may permit peptide groups of the same molecule to interact with different receptors. PEG may also improve the water solubility of the compounds, thereby providing more favorable bioavailability and physiological half-life. Improvement in these properties may provide more effective therapy, and can result in subjects taking smaller, more economical, more convenient, and less frequent doses.

In various embodiments, deficiencies or undesired properties in peptides or peptide analogs may be overcome with the compounds of the present invention. For example, without wishing to be bound by theory, an affinity to anti-thrombin may be considered disadvantageous. Thus, in various embodiments, the compounds of the invention do not have specific binding affinity for anti-thrombin, or do not have an increased binding affinity to anti-thrombin. In one embodiment, comparisons for anti-thrombin binding are made with respect to enfuvirtide.

In one aspect, compounds according to the invention may be represented by the formula:

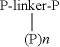

wherein P is a peptide modified to include a covalent bond to said linker, or P is a peptide analog, and n is 0 or 1. In various embodiments, the peptide analog has at least about 50% of the bioactivity of the non-modified peptide. For example, in certain embodiments, the peptide analog differs from the parent peptide in that the peptide analog contains at least one cysteine more than the parent peptide, or at least one modified natural amino acid, at least one modified or unmodified non-natural amino acid, at least one amino acid analog, or combinations thereof. Where n=0, compounds according to the invention may be represented by the formula:

P-linker-P.

In various embodiments, compounds, according to the invention may contain two or three groups derived from enfuvirtide, joined by a linker. For example, compounds according to the invention may be represented by the formula:

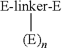

wherein E is enfuvirtide modified to include a covalent bond to said linker, or E is an enfuvirtide analog, and n is 0 or 1. In various embodiments, the enfuvirtide analog has at least about 50% of the bioactivity of enfuvirtide. For example, in certain embodiments, the enfuvirtide analog contains at least one cysteine, at least one modified natural amino acid, at least one modified or unmodified non-natural amino acid, at least one amino acid analog, or combinations thereof. Where n=0, compounds according to the invention may be represented by the formula:

E-linker-E.

In various embodiments, compounds according to the invention may contain two or three groups derived from exenatide, joined by a linker. For example, compounds according to the invention may be represented by the formula:

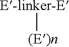

wherein E′ is exenatide modified to include a covalent bond to said linker, or E′ is an exenatide analog, and n is 0 or 1. In various embodiments, the exenatide analog has at least about 50% of the bioactivity of exenatide. For example, in certain embodiments, the exenatide analog contains at least one cysteine more than exenatide, at least one modified natural amino acid, at least one modified or unmodified non-natural amino acid, at least one amino acid analog, or combinations thereof. Where n=0, compounds according to the invention may be represented by the formula:

E′-linker-E′.

The linker, in addition to providing a covalent connection between the two or three appended groups, may also provide functional characteristics. For example, the linker may contain groups to enhance solubility and pharmacokinetic properties. In various embodiments, the linker contains a group selected from polyethylene glycol, polypropylene glycol, polyamine, polyamide, polyurethane, polyester, and combinations thereof.

In some embodiments, the invention provides a compound of the formula (I):

Pep-S-L-(CH₂CH₂O)ₙ—CH₂CH₂-L-S-Pep,

wherein
each Pep is independently a peptide of the sequence:
X¹-(X)^{m}-X^{m+1}, wherein:
X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy;
each (X) independently represents an amino acid or G;
m is the number of independent (X) groups ranging from 0-1000;
G is a sulfur-containing moiety selected from Cys and a non-natural amino acid, such that each Pep includes at least one G;
and X^{m+1} is —N(R^{A})₂, -G-N(R^{A})₂, —O(R^{O}), or -G-O(R^{O}) wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl;
   each S is independently the sulfur atom of a G residue;
   each L is a linker group; and
   and n is an integer from 0-1,000;
   or a pharmaceutically-acceptable salt thereof.

In various embodiments, each linker group L independently comprises an electron-withdrawing group and a hydrocarbyl group, and optionally further comprises one or more of a polyethylene glycol group, an ester linkage, an amide linkage, a carbamate linkage, an ether linkage, and an amine linkage.

In some embodiments, the invention provides a compound of the formula (I):

Pep-S-L-(CH₂CH₂O)ₙ—CH₂CH₂-L-S-Pep;

wherein
each Pep is independently a peptide of the sequence:
X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy; X² is Tyr or G; X³ is Thr or G; X⁴ is Ser or G; X⁵ is Leu or G; X⁶ is Ile or G; X⁷ is His or G; X⁸ is Ser or G; X⁹ is Leu or G; X¹⁰ is Ile or G; X¹¹ is Glu or G; X¹² is Glu or G; X¹³ is Ser or G; X¹⁴ is Gln or G; X¹⁵ is Asn or G; X¹⁶ is Gln or G; X¹⁷ is Gln or G; X¹⁸ is Glu or G; X¹⁹ is Lys or G; X²⁰ is Asn or G; X²¹ is Glu or G; X²² is Gln or G; X²³ is Glu or G; X²⁴ is Leu or G; X²⁵ is Leu or G; X²⁶ is Glu or G; X²⁷ is Leu or G; X²⁸ is Asp or G; X²⁹ is Lys or G; X³⁰ is Trp or G; X³¹ is Ala or G; X³² is Ser or G; X³³ is Leu or G; X³⁴ is Trp or G; X³⁵ is Asn or G; X³⁶ is Trp or G; X³⁷ is Phe or G; and X³⁸ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently the sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and n is an integer from 0-1,000, or a pharmaceutically-acceptable salt thereof.

In some embodiments, the invention provides a compound of formula (I):

Pep-S-L-(CH₂CH₂O)ₙ—CH₂CH₂-L-S-Pep,

wherein
each Pep is independently a peptide of the sequence:
X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸-X³⁹-X⁴⁰-X⁴¹, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy; X² is His or G; X³ is Gly or G; X⁴ is Glu or G; X⁵ is Gly or G; X⁶ is Thr or G; X⁷ is Phe or G; X⁸ is Thr or G, X⁹ is Ser or G; X¹⁰ is Asp or G; X¹¹ is Leu or G; X¹² is Ser or G; X¹³ is Lys or G; X¹⁴ is Gln or G; X¹⁵ is Met or G; X¹⁶ is Glu or G; X¹⁷ is Glu or G; X¹⁸ is Glu or G; X¹⁹ is Ala or G; X²⁰ is Val or G; X²¹ is Arg or G; X²² is Leu or G; X²³ is Phe or G; X²⁴ is Ile or G; X²⁵ is Glu or G; X²⁶ is Trp or G; X²⁷ is Leu or G; X²⁸ is Lys or G; X²⁹ is Asn or G; X³⁰ is Gly or G; X³¹ is Gly or G; X³² is Pro or G; X³³ is Ser or G; X³⁴ is Ser or G; X³⁵ is Gly or G; X³⁶ is Ala or G; X³⁷ is Pro or G; X³⁸ is Pro or G; X³⁹ is Pro or G; X⁴⁰ is Ser or G; and X⁴¹ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently the sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and n is an integer from 0-1.000, or a pharmaceutically-acceptable salt thereof.

In alternate embodiments, the invention provides a compound of the formula (IV):

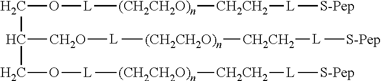

each Pep is independently a peptide of the sequence:
X¹-(X)^{m} X^{m+1}, wherein:
X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl aralkyl, substituted aralkyl, acyl, carbamoyl, or carboalkoxy;
each (X) independently represents an amino acid or G;
m is the number of independent (X) groups ranging from 0-1000;
G is a sulfur-containing moiety selected from Cys and a non-natural amino acid, such that each Pep includes at least one G;
and X^{m+1} is —N(R^{A})₂, -G-N(R^{A})₂, —O(R^{O}), or -G-O(R^{O}) wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl;
   each S is independently the sulfur atom of a G residue;
   each L is a linker group; and
   and n is an integer from 0-1,000;
   or a pharmaceutically-acceptable salt thereof.

In various embodiments, each linker group L independently comprises an electron-withdrawing group and a hydrocarbyl group, and optionally further comprises one or more of a polyethylene glycol group, an ester linkage, an amide linkage, a carbamate linkage, an ether linkage, and an amine linkage.

In alternate embodiments, the invention provides a compound of the formula (IV):

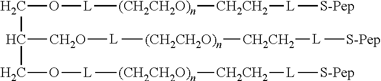

wherein
each Pep is independently a peptide of the sequence:
X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy; X² is Tyr or G; X³ is Thr or G; X⁴ is Ser or G; X⁵ is Leu or G; X⁶ is Ile or G; X⁷ is His or G; X⁸ is Ser or G; X⁹ is Leu or G; X¹⁰ is Ile or G; X¹¹ is Glu or G; X¹² is Glu or G; X¹³ is Ser or G; X¹⁴ is Gln or G; X¹⁵ is Asn or G; X¹⁶ is Gln or G; X¹⁷ is Gln or G; X¹⁸ is Glu or G; X¹⁹ is Lys or G; X²⁰ is Asn or G; X²¹ is Glu or G; X²² is Gln or G; X²³ is Glu or G; X²⁴ is Leu or G; X²⁵ is Leu or G; X²⁶ is Glu or G; X²⁷ is Leu or G; X²⁸ is Asp or G; X²⁹ is Lys or G; X³⁰ is Trp or G; X³¹ is Ala or G; X³² is Ser or G; X³³ is Leu or G; X³⁴ is Trp or G; X³⁵ is Asn or G; X³⁶ is Trp or G; X³⁷ is Phe or G; and X³⁸ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently the sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and n is an integer from 0-1,000, or a pharmaceutically-acceptable salt thereof.

In alternate embodiments, the invention provides a compound of the formula (IV):

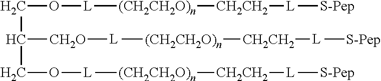

wherein
each Pep is independently a peptide of the sequence:
X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵-X²⁶-X²⁷-X²⁸-X²⁹-X³⁰-X³¹-X³²-X³³-X³⁴-X³⁵-X³⁶-X³⁷-X³⁸-X³⁹-X⁴⁰-X⁴¹, wherein: X¹ is (R^{A})₂N— or (R^{A})₂N-G-, wherein each R^{A} is independently H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, substituted aralkyl, acyl, carbamoyl, or carboalkoxy; X² is His or G; X³ is Gly or G; X⁴ is Glu or G; X⁵ is Gly or G; X⁶ is Thr or G; X⁷ is Phe or G; X⁸ is Thr or G, X⁹ is Ser or G; X¹⁰ is Asp or G; X¹¹ is Leu or G; X¹² is Ser or G; X¹³ is Lys or G; X¹⁴ is Gln or G; X¹⁵ is Met or G; X¹⁶ is Glu or G; X¹⁷ is Glu or G; X¹⁸ is Glu or G; X¹⁹ is Ala or G; X²⁰ is Val or G; X²¹ is Arg or G; X²² is Leu or G; X²³ is Phe or G; X²⁴ is Ile or G; X²⁵ is Glu or G; X²⁶ is Trp or G; X²⁷ is Leu or G; X²⁸ is Lys or G; X²⁹ is Asn or G; X³⁰ is Gly or G; X³¹ is Gly or G; X³² is Pro or G; X³³ is Ser or G; X³⁴ is Ser or G; X³⁵ is Gly or G; X³⁶ is Ala or G; X³⁷ is Pro or G; X³⁸ is Pro or G; X³⁹ is Pro or G; X⁴⁰ is Ser or G; and X⁴¹ is —N(R^{A})₂, -G-N(R^{A})₂, —O(R^{O}), or -G-O(R^{O}), wherein R^{O} is H, hydrocarbyl, substituted hydrocarbyl, heteroalkyl, substituted heteroalkyl, heterocyclyl, substituted heterocyclyl, heterocyclylalkyl, substituted heterocyclylalkyl, aryl, substituted aryl, aralkyl, or substituted aralkyl, wherein each Pep includes at least one G; G is a sulfur-containing moiety selected from Cys and a non-natural amino acid; each S is independently the sulfur atom of a G residue; L is a linker group, wherein each linker group independently comprises an electron-withdrawing group and a hydrocarbyl group, and optionally further comprises one or more of a polyethylene glycol group, an ester linkage, an amide linkage, a carbamate linkage, an ether linkage, and an amine linkage; and each n is independently an integer from 0-1,000, or a pharmaceutically-acceptable salt thereof.

In an alternate embodiment, a modified or unmodified, natural or unnatural amino acid or analog thereof is inserted between any two X moieties above.

Formula (I) provides compounds comprising two peptide units and a bivalent linker. Formula (IV) provides compounds comprising three peptide units, three bivalent linkers, and a trivalent core. Each bivalent linker is connected to a single peptide unit and to the trivalent core. In some embodiments, the linker is PEG, optionally functionalized for case of formation of covalent bonds. In some embodiments, the trivalent core is 2-hydroxymethyl-1,3-propanediol (the triol). In some embodiments the peptide is enfuvirtide, while in other embodiments, the peptide is exenatide.

In some embodiments, both occurrences of Pep have the same sequence.

In some embodiments, each Pep has one Cys residue.

In some embodiments, n is an integer from 0-100.

In some embodiments, n is an integer from 1-50, or from 5-20, or from 8-15.

In some embodiments, n is 9, 10, 11, 12, or 13. In some embodiments, n is 11.

In some embodiments, each L is independently:

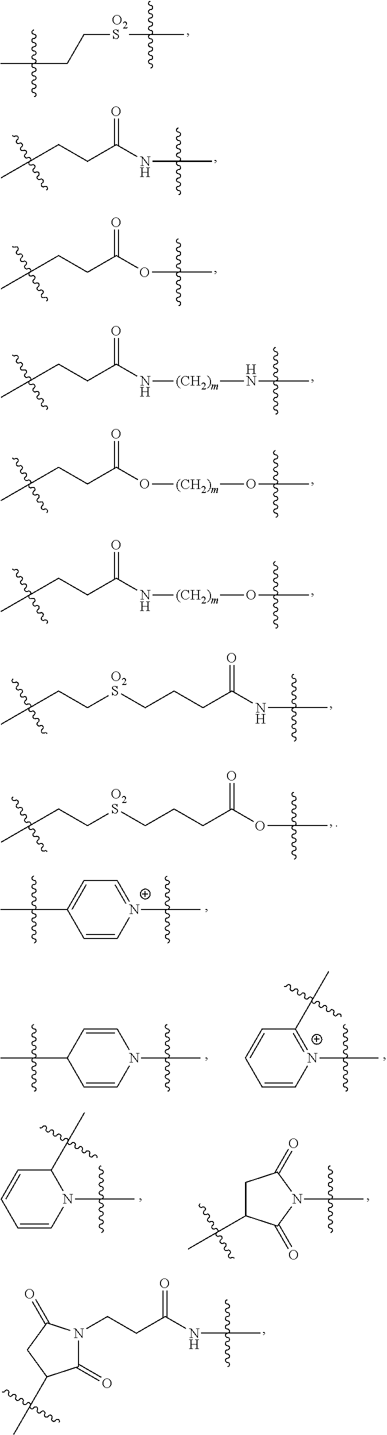

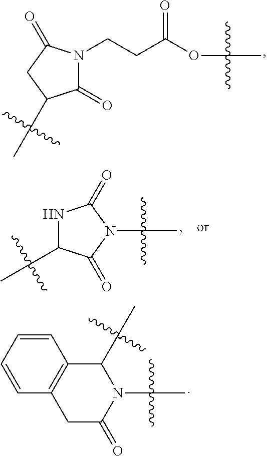 [SMILES: *OC(=O)CCN1C(=O)CC(*)C1=O ; *C1NC(=O)N(*)C1=O ; *C1c2ccccc2CC(=O)N1*]

In some embodiments, each occurrence of L has the same structure.

When Pep is an analog of enfuvirtide, the analog may have various amino acids in common with enfuvirtide. For example, in some embodiments, X¹⁵ is Asn; X¹⁶ is Gln; X¹⁷ is Gln; X¹⁸ is Glu; X¹⁹ is Lys; X²⁰ is Asn; X²¹ is Glu; X²² is Gln; X²³ is Glu; and X²⁴ is Leu. In some embodiments, X¹⁰ is Ile; X¹¹ is Glu; X¹² is Glu; X¹³ is Ser; X¹⁴ is Gln; X²⁵ is Leu; X²⁶ is Glu; X²⁷ is Leu; X²⁸ is Asp; and X²⁹ is Lys. In some embodiments, X⁶ is Ile; X⁷ is His; X⁸ is Ser; X⁹ is Leu; X³⁰ is Trp; X³¹ is Ala; X³² is Ser; and X³³ is Leu. In some embodiments, X⁴ is Ser; X⁵ is Leu; X³⁴ is Trp; and X³⁵ is Asn. In some embodiments, X³ is Thr; and X³⁶ is Trp.

In some embodiments, the compound has a longer period of physiological efficacy than enfuvirtide or exenatide.

In some embodiments, all occurrences of Pep are the same, and are any one of SEQ ID NO: 2-77, or SEQ ID NO: 79-150.

In some embodiments, n is 11 and m is from 2 to 10.

In some embodiments, the compound is:

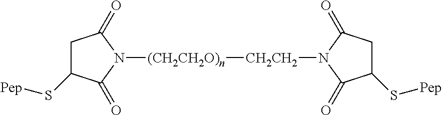

In some embodiments, all occurrences of:

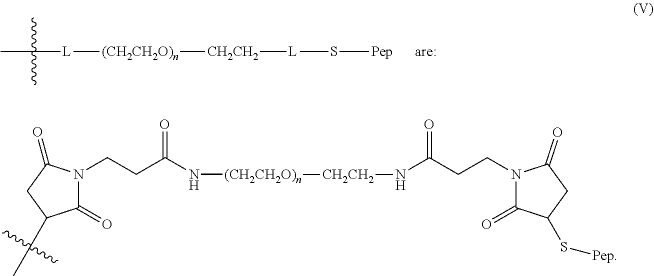

Non-limiting examples of compounds of the invention include:

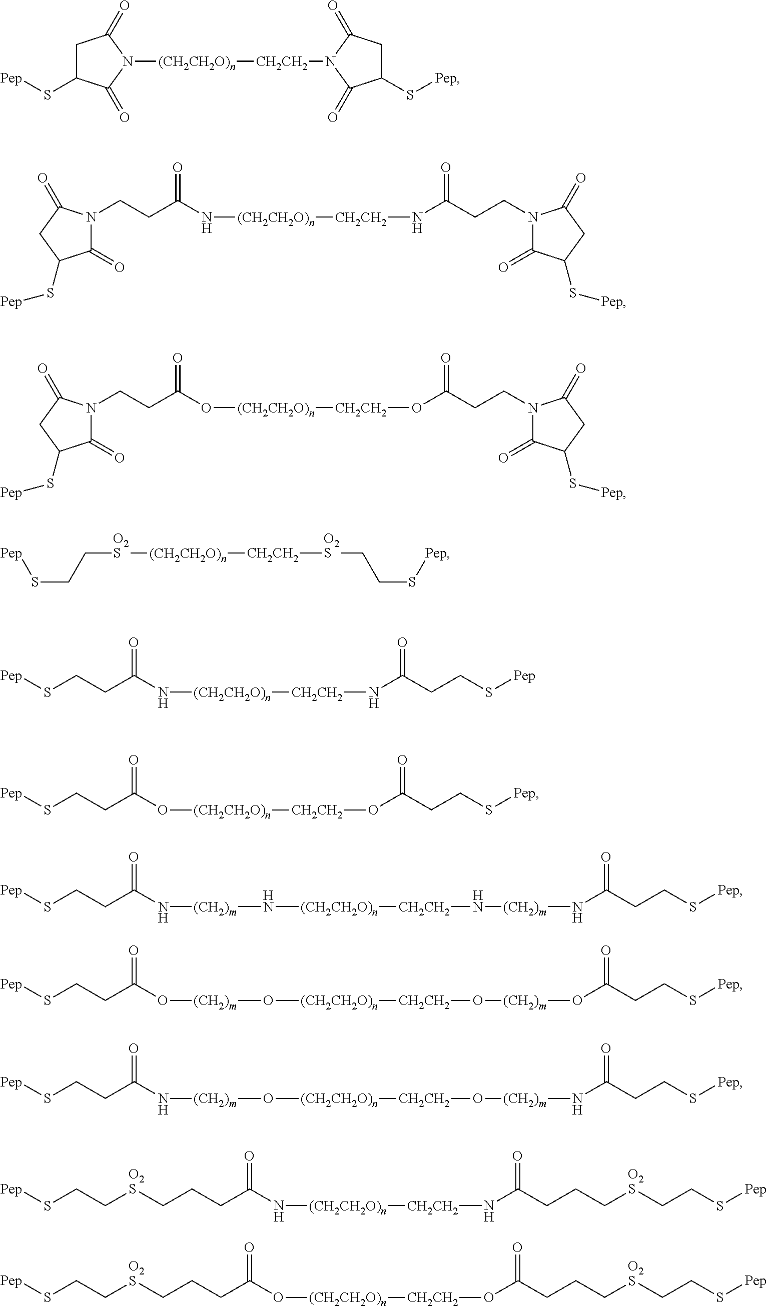

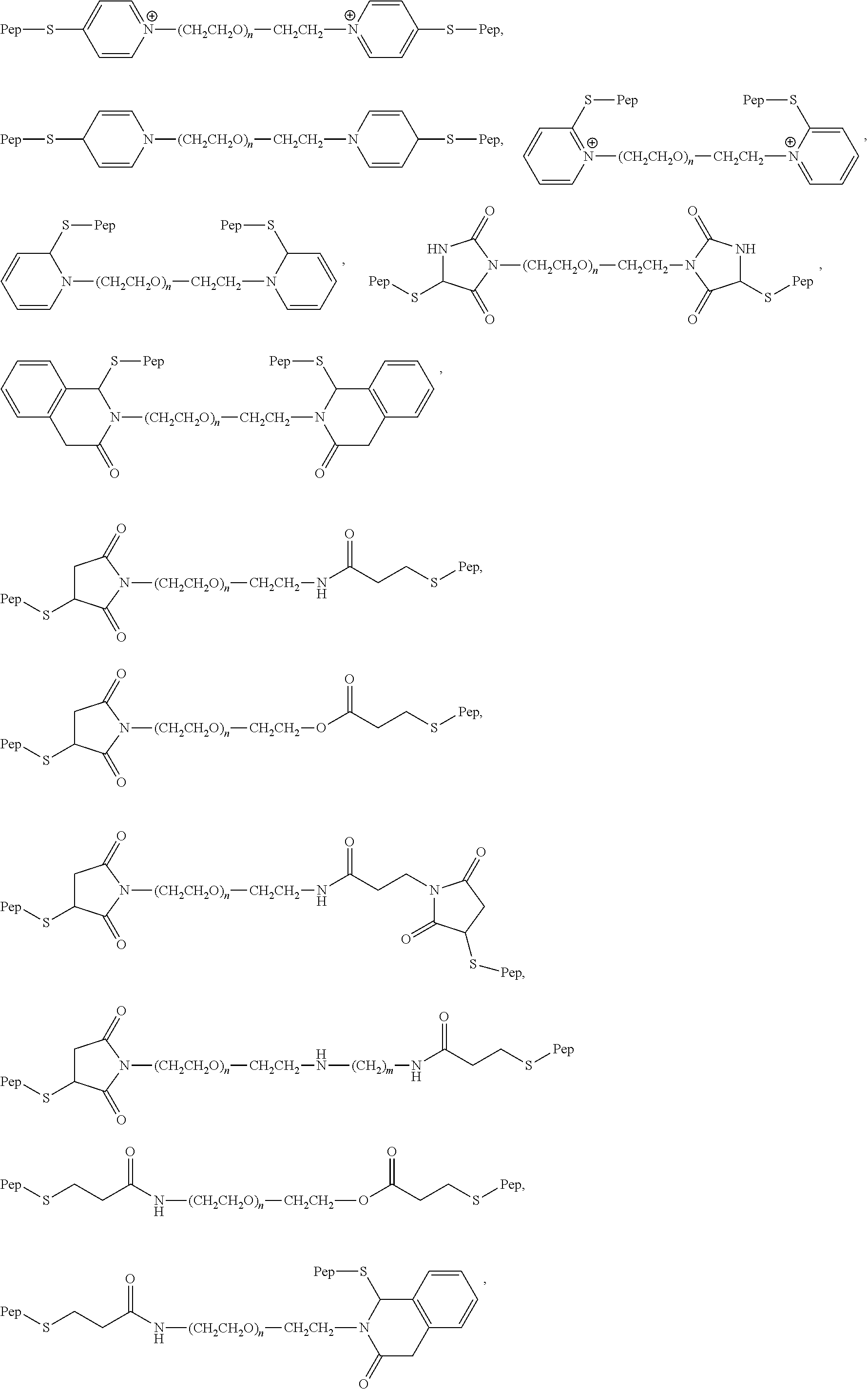

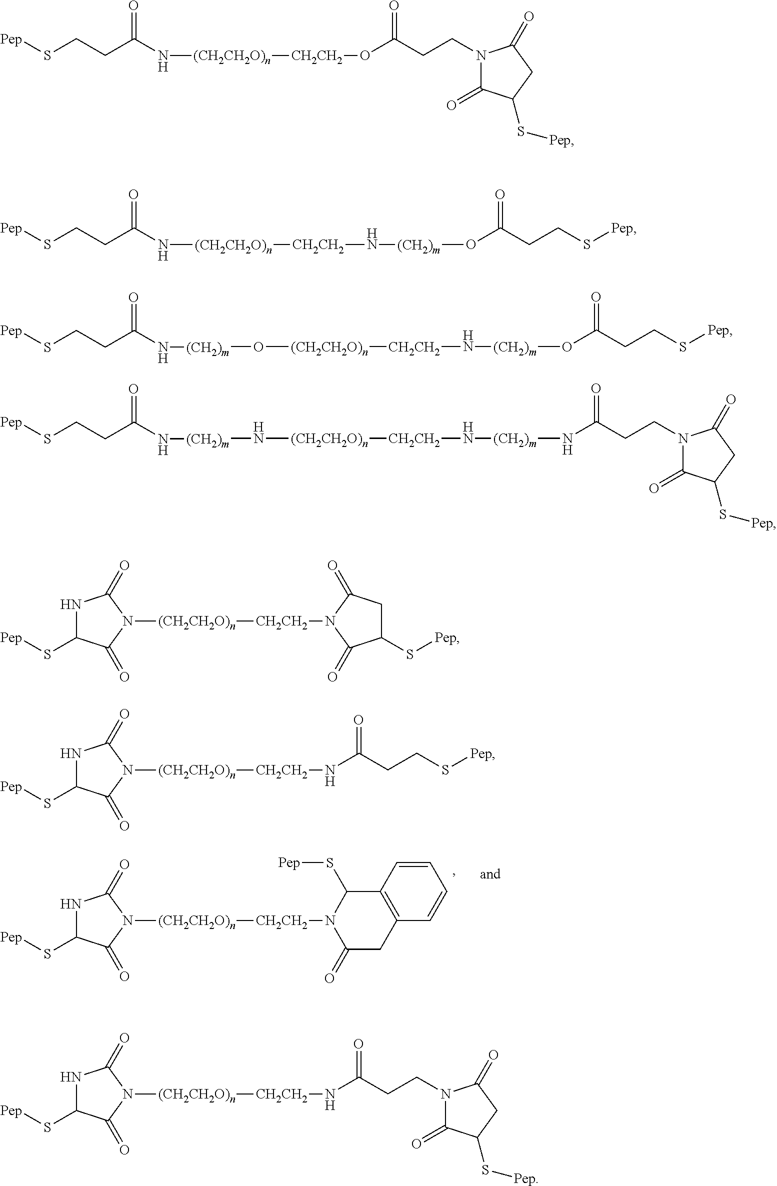

Additional non-limiting examples of compounds of the invention include:

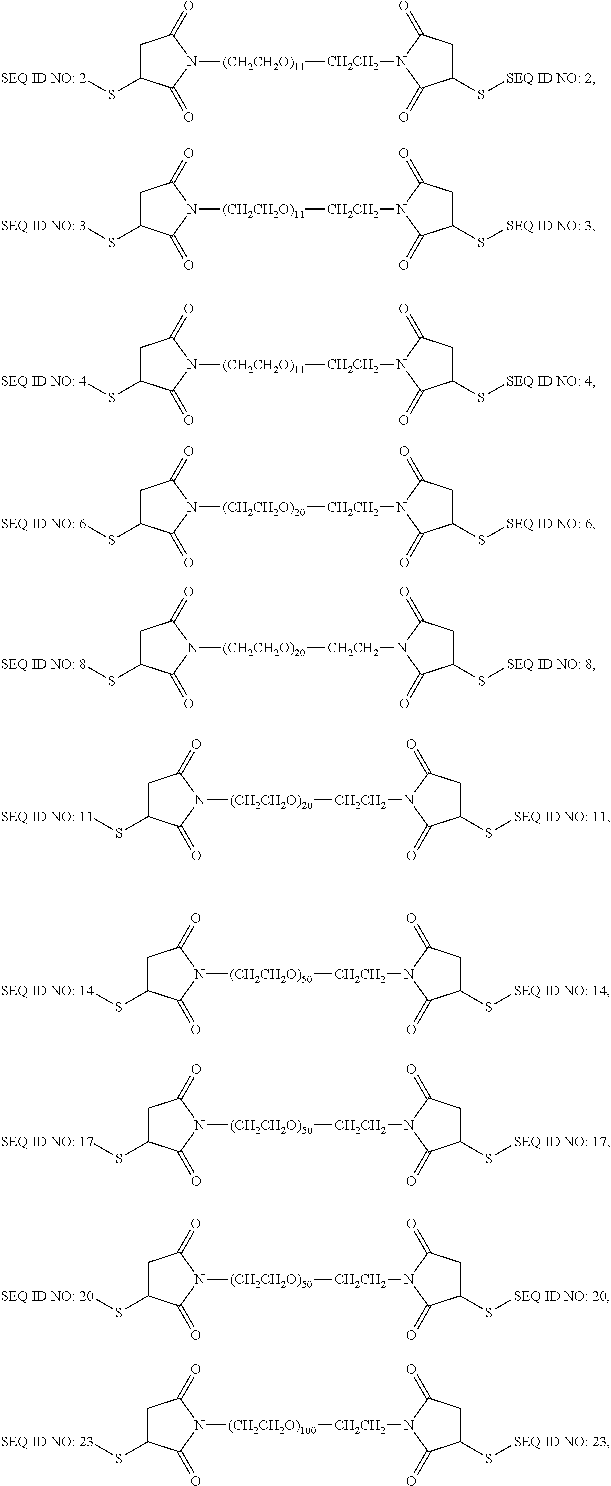

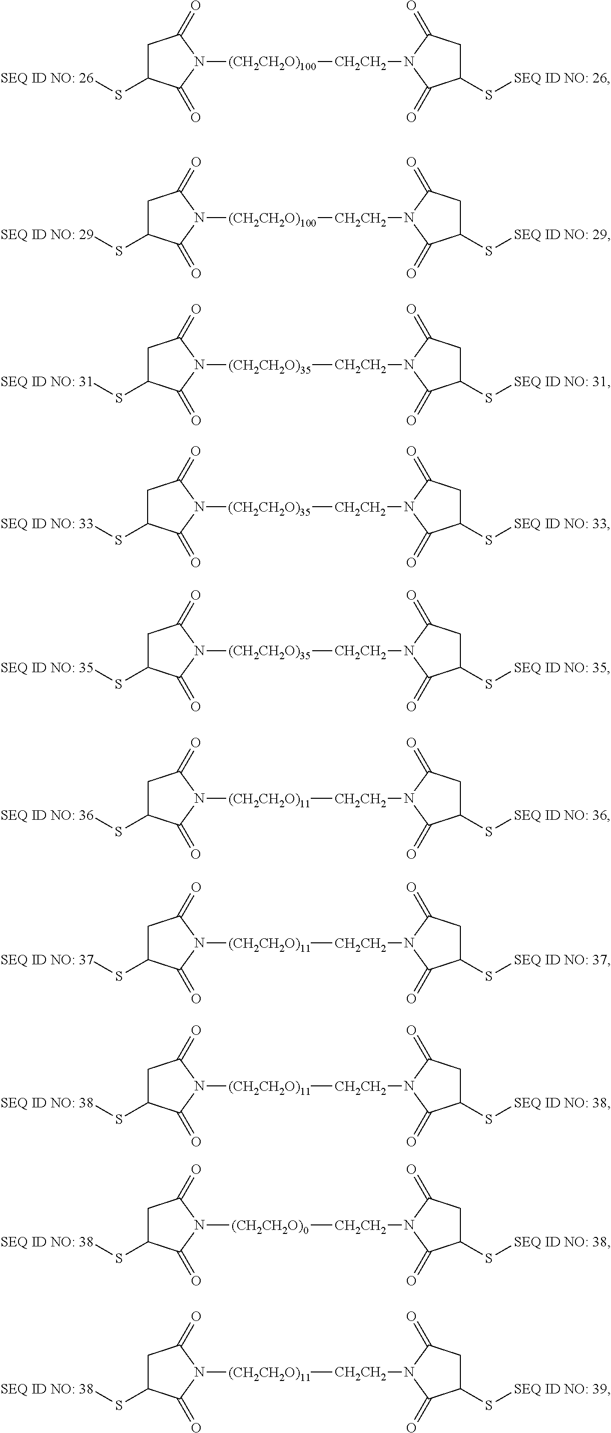

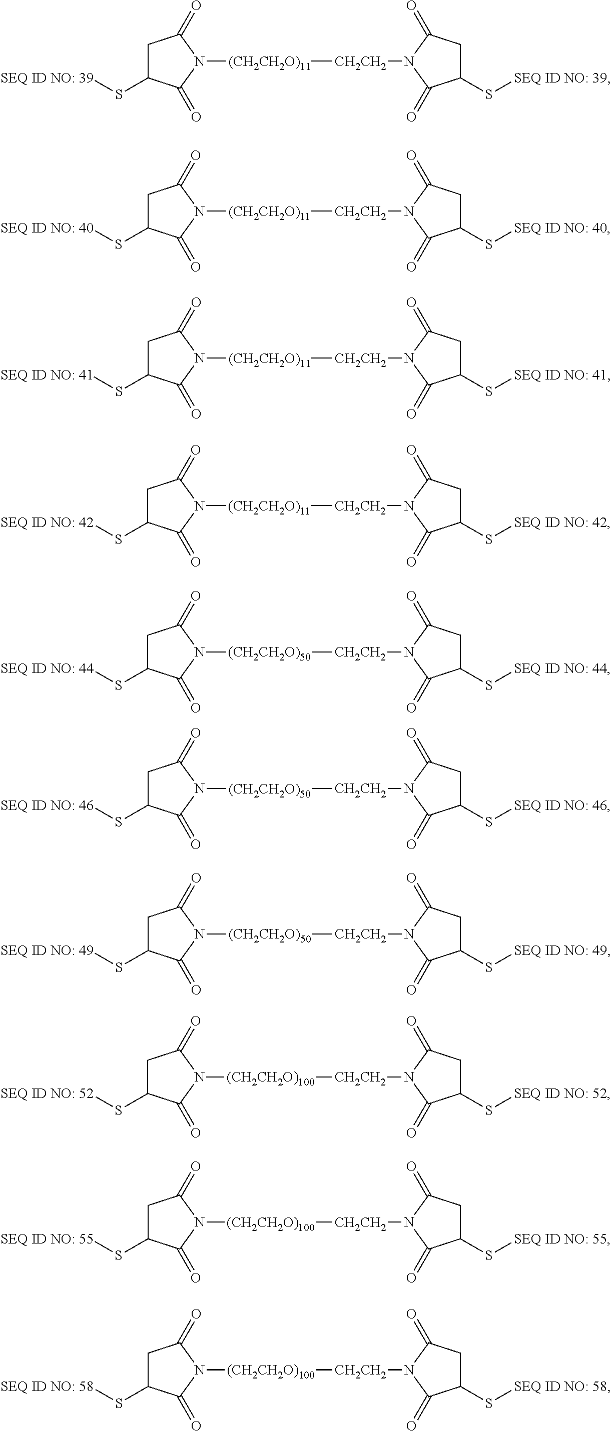

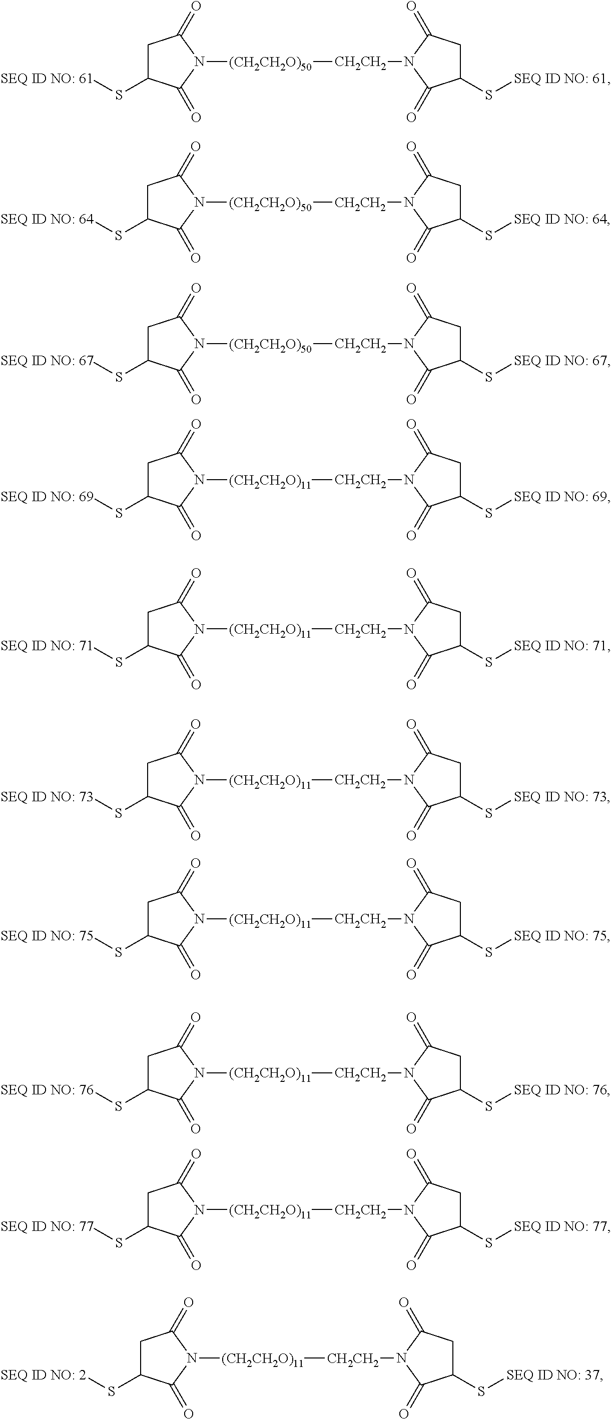

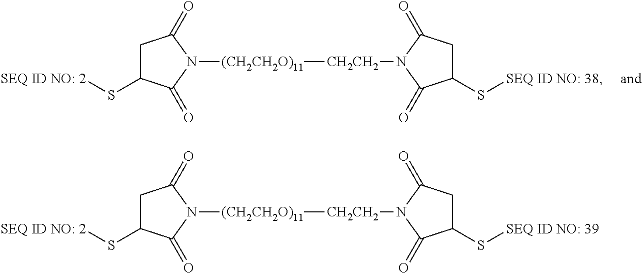

Non-limiting examples of compounds of the invention also include compounds of formula (IV):

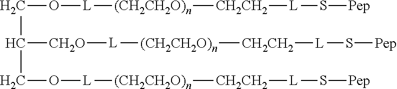

wherein each occurrence of formula (V):

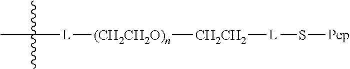

is independently:

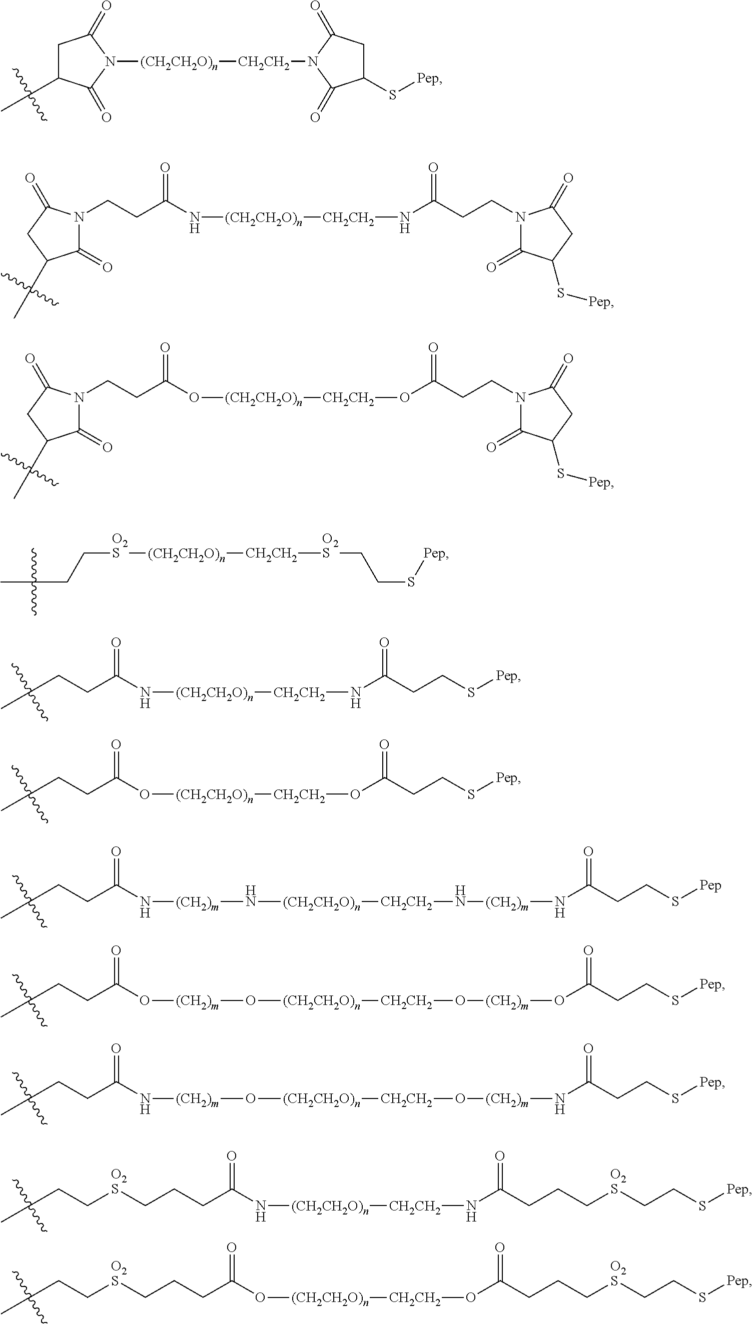

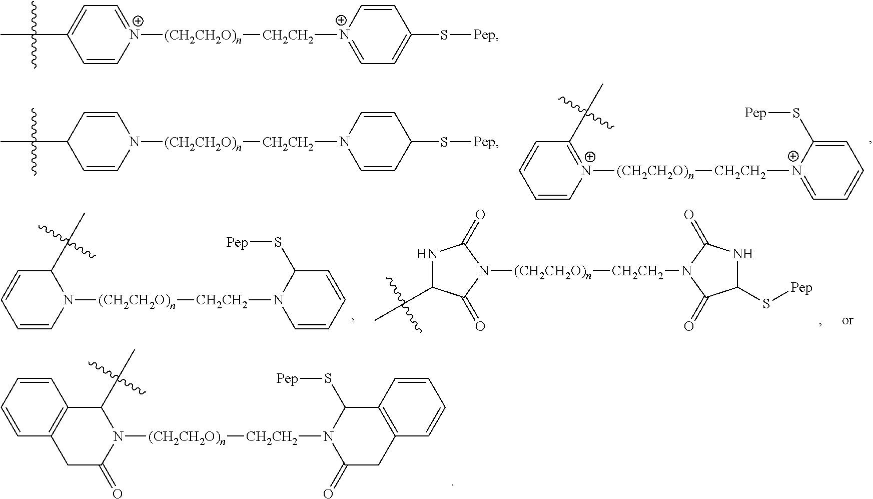

Peptides

In some embodiments, a compound of the invention comprises two peptide groups, each independently connected to a bivalent linker group. In some embodiments, a compound of the invention comprises three peptide groups, each independently connected to a, trivalent linker group, or each independently connected to a bivalent linker group which is further connected to a trivalent core.

In various embodiments, a peptide analog, either alone or when linked according to formulas (I) or (IV), will have bioactivity of at least 50% of the bioactivity of the unmodified peptide, with various embodiments having greater than 60%, greater than 70%, greater than 80%, greater than 90%, or greater than 95% of the bioactivity of the unmodified peptide, all amounts being “about”. Without wishing to be bound by theory, compounds according to Formulas (I) or (IV) may have higher avidity due to the ability to bind to two target molecules simultaneously with the flexibility of the linker, including PEG linkers. Bivalent or trivalent binding may be more stable than monovalent binding, as multiple peptides would have to dissociate simultaneously for a bivalently or trivalently bound molecule to detach from the surface of a receptor on a virus or cell. As such, various compounds according to the invention may have improved stability, greater solubility, and reduced antigenicity, among other advantages. In various embodiments, the peptide analog is an analog of enfuvirtide or exenatide.

The sequence of enfuvirtide is YTSLIHSLIE ESQNQQEKNE QELLELNKWA SLWNWF (SEQ ID NO: 1). The invention provides compounds comprising enfuvirtide analogs wherein the sequence of an enfuvirtide analog differs from the sequence of enfuvirtide by the insertion of at least one cysteine residue or the substitution of at least one residue of SEQ ID NO: 1 with a cysteine residue. The sulfur atom of the cysteine residue side chain may be used to connect the enfuvirtide analog to the linker. Non-limiting examples of the sequences of the present invention are provided in Table 1.

The sequence of exenatide is HGEGTFTSDL SKQMEEEAVR LFIEWLKNGG PSSGAPPPS (SEQ ID NO: 78). The invention provides compounds comprising exenatide analogs wherein the sequence of an exenatide analog differs from the sequence of exenatide by the insertion of at least one cysteine residue or the substitution of at least one residue of SEQ ID NO:78 with a cysteine residue. The sulfur atom of the cysteine residue side chain may be used to connect the exenatide analogs to the linker. Non-limiting examples of the sequences of the present invention are provided in Table 2.

In some embodiments, all the peptide groups of a single molecule of the present invention have the same sequence. In some embodiments, the peptide groups of a single molecule of the present invention do not have the same sequence. In some embodiments, all the peptide groups of a plurality of molecules of the present invention have the same sequence. In some embodiments, not all the peptide groups of a plurality of molecules of the present invention have the same sequence. In some embodiments, a single molecule containing peptide groups that are not of the same sequence has a therapeutic effect that is not the same as that of a molecule containing peptide groups wherein all the peptide groups are of the same sequence. In some embodiments, a plurality of molecules wherein not all of the peptide groups of the plurality of molecules have the same sequence has a therapeutic effect that is not the same as that of a plurality of molecules wherein all of the peptide groups of the plurality of molecules have the same sequence.

Further non-limiting embodiments of the invention include additional peptides and analogs thereof where Pep is selected from the group consisting of arginine vasopressin, AGG01, amylin (IAPP), amyloid beta, avian pancreatic polypeptide (APP), B-type natriuretic peptide (BNP), calcitonin peptides, calcitonin, colistin (polymyxin E), colistin copolymer 1 (Cop-1), cyclosporin, darbepoetin, PDpoetin, eledoisin, enfuvirtide, enkephalin pentapeptides, epoetin, epoetin delta, erythropoietin, exenatide, GHRH 1-24 (Growth Hormone Releasing Hormone 1-24), glucagon, growth hormone, glucagon-like peptide-1 (GLP-1), granulocyte-colony stimulating factor (G-CSF), granulocyte macrophage-colony stimulating factor (GM-CSF), insulin, hGH, interferon, kassinin, lactotripeptides, leptin, lixisenatide, luteinizing-hormone-releasing hormone, methoxy polyethylene glycol-epoetin beta (MIRCERA), neurokinin A, neurokinin B, NPY (NeuroPeptide Y), octreotide, pituitary adenylate cyclase activating peptide (PACAP), parathyroid hormone (PTH), peptide PHI 27 (Peptide Histidine Isoleucine 27), proopiomelanocortin (POMC) peptides, prodynorphin peptides, polymyxins, polymyxin B, PPY (Pancreatic PolYpeptide), PYY (Peptide YY), secretin, Substance P, thrombospondins (TSP), ubiquitin, or VIP (Vasoactive Intestinal Peptide; PHM27). In various embodiments, Pep is selected from peptides for diabetes treatment, such as exenatide, glucagon-like peptide-1 (GLP-1), or lixisenatide.

Salts

The invention provides pharmaceutically-acceptable salts. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base.

In some embodiments, a pharmaceutically-acceptable salt is a metal salt. Metal salts can arise from the addition of an inorganic base to a compound of the invention. The inorganic base consists of a metal cation paired with a basic counterion such as, for example, hydroxide, carbonate, bicarbonate, or phosphate. The metal may be an alkali metal, alkaline earth metal, transition metal, or main group metal. In some embodiments, the metal is lithium, sodium, potassium, cerium, magnesium, manganese, iron, calcium, aluminum, copper, or zinc.

In some embodiments, a pharmaceutically-acceptable salt is an ammonium salt. Ammonium salts can arise from the addition of ammonia or an organic amine to a compound of the invention. In some embodiments, the organic amine is triethylamine, ethanolamine, diethanolamine, triethanolamine, morpholine, piperidine, N-methylpiperidine. N-ethylpiperidine, dibenzylamine, piperazine, pyridine, pyrazine, or pipyrazine.

Acid addition salts can arise from the addition of an acid to a compound of the invention. In some embodiments, the acid is organic. In some embodiments, the acid is inorganic. In other embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, or maleic acid.

Linkers

In general, linkers according to the invention provide a covalent attachment between two or more peptides or peptide analogs. The linker groups of the compounds of the invention can be any chemical moieties suitable for connection between two or more peptide groups. A compound of the invention can have 1, 2, 3, 4, 5, 6, 7, 8, 9, or 10 linker groups. In some embodiments, a compound of the invention has 2 linker groups. In some embodiments, a compound of the invention has 6 linker groups. In some embodiments, a linker group is bifunctional. In some embodiments, a linker group comprises an electron-withdrawing group. In some embodiments, a linker group comprises an electron-withdrawing group and a hydrocarbyl group. In some embodiments, a linker group further comprises one or more of a polyethylene glycol group, an ester linkage, an amide linkage, a carbamate linkage, an ether linkage, and an amine linkage. In some embodiments, a linker group comprises an electron-withdrawing group and a hydrocarbyl group, and optionally further comprises one or more of a polyethylene glycol group, an ester linkage, an amide linkage, a carbamate linkage, an ether linkage, and an amine linkage.

In various embodiments, a linker group arises form a chemical reaction that conjugates a peptide group to a bivalent tether. The linkage can take place through the sulfhydryl of a cysteine residue of the peptide group. The precursor to the linker group is a reactive group comprising a functional group suitable for chemical reaction with the cysteine sulfyhydryl group. In some embodiments, the reactive group is an electrophile. In some embodiments, the reactive group is a Michael acceptor. In some embodiments, the reactive group is an electrophilic aromatic group. In some embodiments, the reactive group is an electrophilic heterocycle. In some embodiments, the reactive group comprises a leaving group. In some embodiments, the reactive group comprises an imine. In some embodiments, the reactive group comprises an iminium group. In some embodiments, a reactive group comprises a multiple bond in conjugation with an electron-withdrawing group. In some embodiments, a reactive group further comprises one or more of a hydrocarbyl group, a polyethylene glycol group, an ester linkage, an amide linkage, a carbamate linkage, an ether linkage, and an amine linkage. In some embodiments, a reactive group comprises a multiple bond in conjugation with an electron-withdrawing group, and optionally further comprises one or more of a hydrocarbyl group, a polyethylene glycol group, an ester linkage, an amide linkage, a carbamate linkage, an ether linkage, and an amine linkage.

Various compounds of the present invention comprise one or more PEG groups. A PEG group is given by formula (IX):

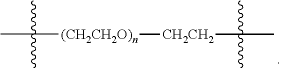

The water-solubility and pharmacokinetic properties of the compounds can be modulated by selecting different lengths or sizes for the PEG groups. In some embodiments, the therapeutic efficacy of a molecule can be modulated by selecting different lengths or sizes for the PEG groups.

In some embodiments, all the PEG groups of a molecule of the invention have the same length. In some embodiments, all the PEG groups of a molecule of the invention have about the same length. In some embodiments, not all the PEG groups of a molecule of the invention have the same length. In some embodiments, all the PEG groups of a plurality of molecules of the present invention have the same length. In some embodiments, all the PEG groups of a plurality of molecules of the present invention have about the same length. In some embodiments, not all the PEG groups of a plurality of molecules of the present invention have the same length. In some embodiments a single molecule containing PEG groups that are not all of the same length has a therapeutic effect that is not the same as that of a molecule containing PEG groups wherein all the PEG groups are of the same length. In some embodiments, a plurality of molecules wherein not all of the PEG groups of the plurality of molecules have the same length has a therapeutic effect that is not the same as that of a plurality of molecules wherein all the PEG groups of the plurality of molecules have the same length.

In some embodiments, the size of a PEG group is measured in the number of ethylene glycol units. In some embodiments, n is 5.000. In some embodiments, n is about 5,000. In some embodiments, n is 2,500. In some embodiments, n is about 2,500. In some embodiments, n is 1,000. In some embodiments, n is about 1,000. In some embodiments, n is 500. In some embodiments, n is about 500. In some embodiments, n is 250. In some embodiments, n is about 250. In some embodiments, n is 100. In some embodiments, n is about 100. In some embodiments, n is 50. In some embodiments, n is about 50. In some embodiments, n is 25. In some embodiments, n is about 25. In some embodiments, n is 10. In some embodiments, n is about 10. In some embodiments, n is from 1 to 5.000. In some embodiments, n is from 1 to about 5,000. In some embodiments, n is from 1 to 2,500. In some embodiments, n is from 1 to about 2,500. In some embodiments, n is from 1 to 1.000. In some embodiments, n is from 1 to about 1.000. In some embodiments, n is from 1 to 500. In some embodiments, n is from 1 to about 500. In some embodiments, n is from 1 to 250. In some embodiments, n is from 1 to about 250. In some embodiments, n is from 1 to 100. In some embodiments, n is from 1 to about 100. In some embodiments, n is from 1 to 50. In some embodiments, n is from 1 to about 50. In some embodiments, n is from 1 to 25. In some embodiments, n is from 1 to about 25. In some embodiments, n is 1, 2, 3, 4, 5, 6, 7, 8, 9, 10, 11, 12, 13, 14, 15, 16, 17, 18, 19, 20, 21, 22, 23, 24, or 25. In some embodiments, n is from 0 to 5.000. In some embodiments, n is from 0 to about 5.000. In some embodiments, n is from 0 to 2.500. In some embodiments, n is from 0 to about 2.500. In some embodiments, n is from 0 to 1.000. In some embodiments, n is from 0 to about 1.000. In some embodiments, n is from 0 to 500. In some embodiments, n is from 0 to about 500. In some embodiments, n is from 0 to 250. In some embodiments, n is from 0 to about 250. In some embodiments, n is from 0 to 100. In some embodiments, n is from 0 to about 100. In some embodiments, n is from 0 to 50. In some embodiments, n is from 0 to about 50. In some embodiments, n is from 0 to 25. In some embodiments, n is from 0 to about 25. In some embodiments, n is 0, 1, 2, 3, 4, 5, 6, 7, 8, 9, 10, 11, 12, 13, 14, 15, 16, 17, 18, 19, 20, 21, 22, 23, 24, or 25. In some embodiments, n is 0. In some embodiments, n is 1. In some embodiments, n is 10. In some embodiments, n is 11. In some embodiments, n is 12.

In some embodiments, the size of a PEG group is measured in molecular mass. In some embodiments, the molecular mass of a PEG group is about 500,000 Daltons, about 450,000 Daltons, about 400.000 Daltons, about 350,000 Daltons, about 300,000 Daltons, about 250,000 Daltons, about 200,000 Daltons, about 180.000 Daltons, about 160,000 Daltons, about 140,000 Daltons, about 120,000 Daltons, about 100,000 Daltons, about 90,000 Daltons, about 80,000 Daltons, about 70,000 Daltons, about 60,000 Daltons, about 50,000 Daltons, about 45,000 Daltons, about 40.000 Daltons, about 35,000 Daltons, about 30,000 Daltons, about 25.000 Daltons, about 20,000 Daltons, about 18,000 Daltons, about 16,000 Daltons, about 14,000 Daltons, about 12,000 Daltons, about 10.000 Daltons, about 9,000 Daltons, about 8,000 Daltons, about 7,000 Daltons, about 6,000 Daltons, about 5,000 Daltons, about 4,500 Daltons, about 4,000 Daltons, about 3,500 Daltons, about 3,000 Daltons, about 2,500 Daltons, about 2,000 Daltons, about 1,500 Daltons, about 1,000 Daltons, about 900 Daltons, about 800 Daltons, about 700 Daltons, about 600 Daltons, about 500 Daltons, about 400 Daltons, about 300 Daltons, about 250 Daltons, about 200 Daltons, about 150 Daltons, about 100 Daltons, or about 50 Daltons.

In some embodiments, the molecular mass of a PEG group is 440526 Daltons, 396,474 Daltons, 352,421 Daltons, 308.369 Daltons, 264,316 Daltons, 220,263 Daltons, 198,237 Daltons, 176,211 Daltons, 154,184 Daltons, 132,158 Daltons, 110,131 Daltons, 88,105 Daltons, 79,295 Daltons, 70,484 Daltons, 61,674 Daltons, 52,863 Daltons, 44,053 Daltons, 39,647 Daltons, 35,242 Daltons, 30,837 Daltons, 26,432 Daltons, 22,026 Daltons, 19,824 Daltons, 17,621 Daltons, 15,418 Daltons, 13,216 Daltons, 11,013 Daltons, 8,811 Daltons, 8,370 Daltons, 7,929 Daltons, 7,489 Daltons, 7.048 Daltons, 6,608 Daltons, 6.167 Daltons, 5.727 Daltons, 5,286 Daltons, 4.846 Daltons, 4,405 Daltons, 4,185 Daltons, 3,965 Daltons, 3,744 Daltons, 3,524 Daltons, 3,304 Daltons, 3,084 Daltons, 2.863 Daltons, 2,643 Daltons, 2,423 Daltons, 2.203 Daltons, 1,982 Daltons, 1,762 Daltons, 1,542 Daltons, 1.322 Daltons, 1,101 Daltons, 1.057 Daltons, 1.013 Daltons, 969 Daltons, 925 Daltons, 881 Daltons, 837 Daltons, 793 Daltons, 749 Daltons, 705 Daltons, 661 Daltons, 617 Daltons, 573 Daltons, 529 Daltons, 485 Daltons, 441 Daltons, 396 Daltons, 352 Daltons, 308 Daltons, 264 Daltons, 220 Daltons, 176 Daltons, 132 Daltons, 88 Daltons, or 44. In some embodiments, the molecular mass of a PEG group is about 440526 Daltons, about 396.474 Daltons, about 352,421 Daltons, about 308,369 Daltons, about 264,316 Daltons, about 220.263 Daltons, about 198,237 Daltons, about 176,211 Daltons, about 154,184 Daltons, about 132,158 Daltons, about 110,131 Daltons, about 88,105 Daltons, about 79,295 Daltons, about 70,484 Daltons, about 61,674 Daltons, about 52,863 Daltons, about 44,053 Daltons, about 39,647 Daltons, about 35,242 Daltons, about 30.837 Daltons, about 26,432 Daltons, about 22,026 Daltons, about 19,824 Daltons, about 17,621 Daltons, about 15,418 Daltons, about 13,216 Daltons, about 11,013 Daltons, about 8,811 Daltons, about 8.370 Daltons, about 7,929 Daltons, about 7,489 Daltons, about 7,048 Daltons, about 6,608 Daltons, about 6,167 Daltons, about 5,727 Daltons, about 5,286 Daltons, about 4,846 Daltons, about 4,405 Daltons, about 4,185 Daltons, about 3,965 Daltons, about 3,744 Daltons, about 3,524 Daltons, about 3,304 Daltons, about 3.084 Daltons, about 2.863 Daltons, about 2.643 Daltons, about 2.423 Daltons, about 2,203 Daltons, about 1.982 Daltons, about 1,762 Daltons, about 1,542 Daltons, about 1.322 Daltons, about 1,101 Daltons, about 1.057 Daltons, about 1,013 Daltons, about 969 Daltons, about 925 Daltons, about 881 Daltons, about 837 Daltons, about 793 Daltons, about 749 Daltons, about 705 Daltons, about 661 Daltons, about 617 Daltons, about 573 Daltons, about 529 Daltons, about 485 Daltons, about 441 Daltons, about 396 Daltons, about 352-Daltons, about 308 Daltons, about 264 Daltons, about 220 Daltons, about 176 Daltons, about 132 Daltons, about 88 Daltons, or about 44 Daltons.

Non-limiting examples of linker groups and reactive groups of the invention are illustrated in Table 3. Each row of Table 3 provides a single non-limiting example of the type of reactive group that can give rise to a corresponding linker group.

TABLE 3

| Linker Group | Reactive Group |
|---|---|
| 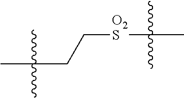 [SMILES: *CCS(*)(=O)=O] | 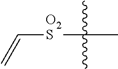 [SMILES: *S(=O)(=O)C=C] |
| 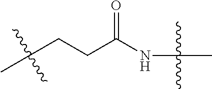 [SMILES: *CCC(=O)N*] | 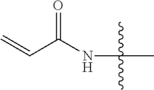 [SMILES: *NC(=O)C=C] |
| 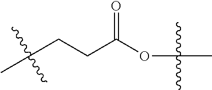 [SMILES: *CCC(=O)O*] | 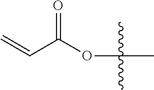 [SMILES: *OC(=O)C=C] |
| 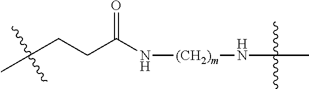 | 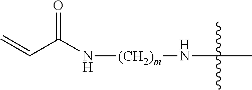 |
| 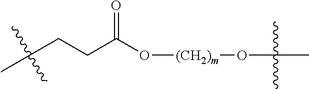 | 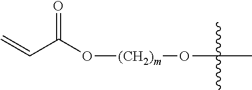 |
| 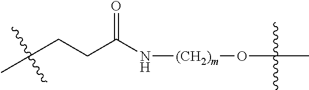 | 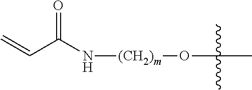 |
| 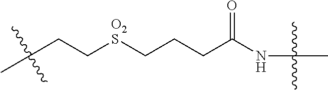 [SMILES: *CCS(=O)(=O)CCCC(=O)N*] | 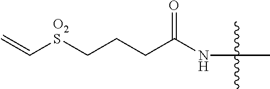 [SMILES: *NC(=O)CCCS(=O)(=O)C=C] |
| 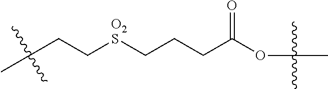 [SMILES: *CCS(=O)(=O)CCCC(=O)O*] | 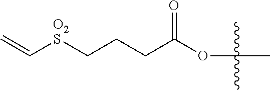 [SMILES: *OC(=O)CCCS(=O)(=O)C=C] |
| 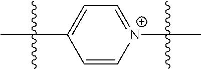 [SMILES: *c1cc[n+](*)cc1] | 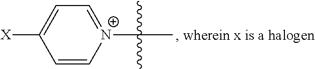 |
| 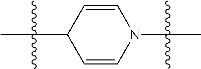 [SMILES: *C1C=CN(*)C=C1] | 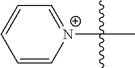 [SMILES: *[n+]1ccccc1] |
| 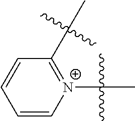 [SMILES: *c1cccc[n+]1*] | 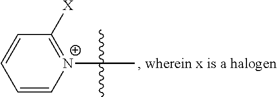 |
| 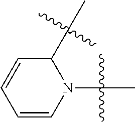 [SMILES: *C1C=CC=CN1*] | 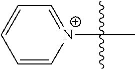 [SMILES: *[n+]1ccccc1] |
| 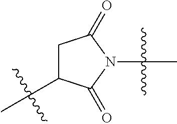 [SMILES: *C1CC(=O)N(*)C1=O] | 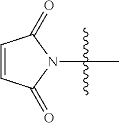 [SMILES: *N1C(=O)C=CC1=O] |
| 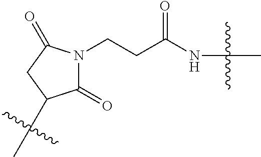 [SMILES: *NC(=O)CCN1C(=O)CC(*)C1=O] | 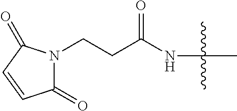 [SMILES: *NC(=O)CCN1C(=O)C=CC1=O] |
| 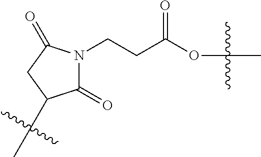 [SMILES: *OC(=O)CCN1C(=O)CC(*)C1=O] | 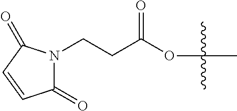 [SMILES: *OC(=O)CCN1C(=O)C=CC1=O] |
| 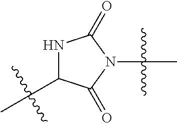 [SMILES: *C1NC(=O)N(*)C1=O] | 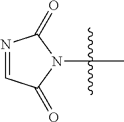 [SMILES: *N1C(=O)C=NC1=O] |
| 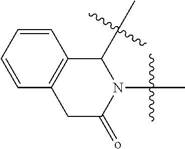 | 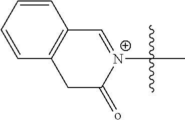 |

Methods of Making Compounds

Compounds of the invention can be made by any synthetic procedures known in the art. For teachings of synthetic organic chemistry theories, methods, strategies, and techniques, see March, ADVANCED ORGANIC CHEMISTRY 4^{th} Ed., (Wiley 1992); Carey and Sundberg, ADVANCED ORGANIC CHEMISTRY 4^{h} Ed., Vols. A and B (Plenum 2000, 2001): and Green and Wuts, PROTECTIVE GROUPS IN ORGANIC SYNTHESIS 3^{rd} Ed., (Wiley 1999), each of which is hereby incorporated by reference in its entirety for the teachings of synthetic organic chemistry theories, methods, strategies, and techniques.

The synthetic reactions can be monitored by any technique known in the art, for example, thin layer chromatography (TLC), mass spectrometry (MS), or high performance liquid chromatography (HPLC). Methods of MS include low resolution MS, high resolution MS, fast atom bombardment (FAB), electrospray (ES), and matrix-assisted laser desorption/ionization (MALDI). Products of the reactions can be analyzed by any technique known to one of skill in the art, including TLC, MS, HPLC, liquid chromatography/mass spectrometry (LCMS), nuclear magnetic resonance (NMR, for ¹H, ¹³C, and heteronuclei), infrared (IR), ultraviolet/visible light spectrophotometry (UV/VIS), melting point, optical rotation, and combustion.

Products of the reactions can be isolated and purified by any isolation or purification technique known to one of skill in the art, including extraction, filtration, silica gel chromatography (either ordinary or reverse phase), HPLC, preparative HPLC, TLC, preparative TLC, crystallization, and size exclusion chromatography. Peptides of the invention can be sequenced by MS techniques known to one of skill in the art.

The invention provides a method of making a compound of the formula (I) as described herein, the method comprising contacting a compound of the formula (II):

RG-(CH₂CH₂O)ₙ—CH₂CH₂-RG

with a compound of the formula (III):

Pep-S—H

wherein each Pep, n, and S are independently as described herein, and further wherein RG is a reactive group, wherein each reactive group independently comprises a multiple bond in conjugation with an electron-withdrawing group, and optionally further comprises one or more of a hydrocarbyl group, a polyethylene glycol group, an ester linkage, an amide linkage, a carbamate linkage, an ether linkage, and an amine linkage. In various embodiments, the Pep group is enfuvirtide or an enfuvirtide analog. Alternatively, the Pep group is exenatide or an exenatide analog.

In some embodiments, the invention provides a method of making a compound of the formula (IV) comprising contacting a compound of the formula (VI):

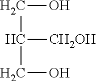

with a compound of the formula (II):

RG-(CH₂CH₂O)ₙ—CH₂CH₂-RG

thereby providing a compound of the formula (VII):

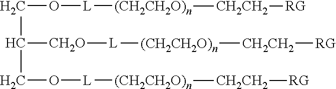

wherein each n and L are independently as described herein, and further wherein RG is a reactive group, wherein each reactive group independently comprises a multiple bond in conjugation with an electron-withdrawing group, and optionally further comprises one or more of a hydrocarbyl group, a polyethylene glycol group, an ester linkage, an amide linkage, a carbamate linkage, an ether linkage, and an amine linkage. The method further comprises contacting a compound of formula (VII) with a compound of the formula (III):

Pep-S—H

thereby providing a compound of the formula (IV), wherein Pep is as described herein. In various embodiments, the Pep group is enfuvirtide or an enfuvirtide analog. Alternatively, the Pep group is exenatide or an exenatide analog.

In some embodiments, the invention provides a method of making a compound of the formula (IV) comprising contacting a compound of the formula (II):

RG-(CH₂CH₂O)ₙ—CH₂CH₂-RG

with a compound of the formula (III):

Pep-S—H

thereby providing a compound of the formula (VIII):

RG-(CH₂CH₂O)ₙ—CH₂CH₂-L-S-Pep

wherein each n, L, S, and Pep are independently as described herein, and further wherein RG is a reactive group, wherein each reactive group independently comprises a multiple bond in conjugation with an electron-withdrawing group, and optionally further comprises one or more of a hydrocarbyl group, a polyethylene glycol group, an ester linkage, an amide linkage, a carbamate linkage, an ether linkage, and an amine linkage. The method further comprises contacting a compound of formula (VIII) with a compound of formula (VI):

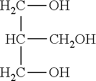

thereby providing a compound of formula (IV). In various embodiments, the Pep group is enfuvirtide or an enfuvirtide analog. Alternatively, the Pep group is exenatide or an exenatide analog.

In some embodiments, each RG is independently:

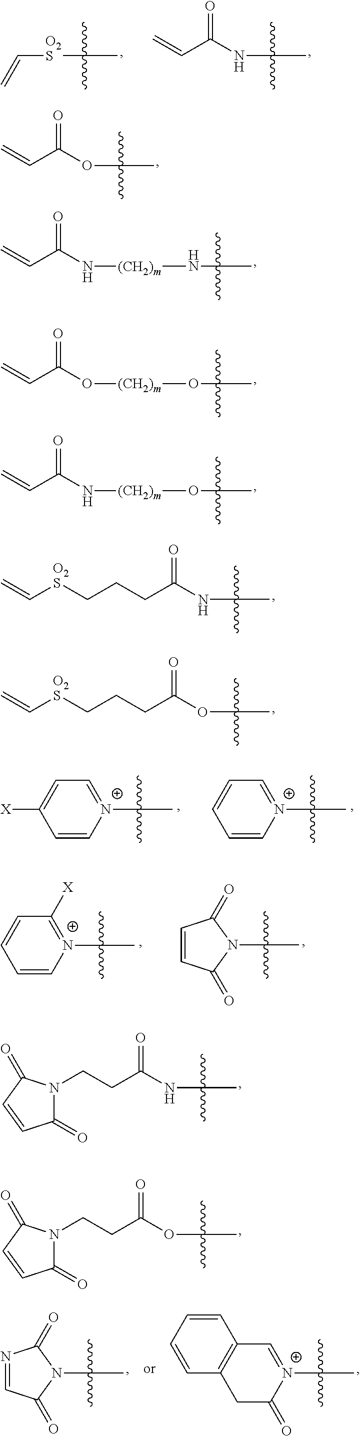

wherein each x is independently a halogen.

In some embodiments, each occurrence of RG has the same structure.

Non-limiting examples of starting materials suitable for the synthesis of bifunctional electrophiles of Formula (II):

RG-(CH₂CH₂O)ₙ—CH₂CH₂-RG (II),

include PEG (HO—(CH₂CH₂O)ₙ—CH₂CH₂—OH), and polyoxyethylene bis(amine) (POEBA) (H₂N—(CH₂CH₂O)ₙ—CH₂CH₂—NH₂), which comprises a PEG group. Both starting materials are known in the art. The termini of PEG are suitable for conjugation to a linker group by methods known in the art, for example, acylation, alkylation, esterification and etherification. The termini of POEBA are suitable for conjugation to a linker group by methods known in the art, for example, acylation, alkylation, amidation and amination.

Non-limiting examples of acylating PEG or POEBA are described herein. The bifunctional starting material is acylated with an acylating agent in a suitable solvent in the presence of a base, and optionally in the presence of a catalyst. In some embodiments, the acylating agent is an acid halide, such as an acid chloride, or an acid anhydride. Non-limiting examples of suitable solvents include tetrahydrofuran (THF), ether (Et₂O), glyme, diglyme, tetraglyme, dichloromethane (DCM), chloroform (CHCl₃), carbon tetrachloride (CCl₄), and acetonitrile (MeCN). Non-limiting examples of suitable bases include triethylamine (TEA), diisopropylethylamine (DIEA), pyridine, 2,6-lutidine, 2,6-di-t-butyl-4-methylpyridine, lithium carbonate, sodium carbonate, potassium carbonate, cesium carbonate, lithium bicarbonate, sodium bicarbonate, potassium bicarbonate, and cesium bicarbonate. Non-limiting examples of suitable catalysts include N,N-dimethylaminopyridine (DMAP). In some embodiments, the acylating agent is formed by contacting a carboxylic acid with a carbodiimide reagent such as dicyclohexylcarbodiimide (DCI), diisopropylcarbodiimide (DIC), or 1-ethyl-3-(3-dimethylaminopropyl) carbodiimide (EDAC) in the presence of a suitable catalyst, such as N-hydroxysuccinimide (HOSu), 1-hydroxybenzotriazole (HOBt), or 1-hydroxy-7-azabenzotriazole (HOAt).

In a representative example, the bifunctional starting material (PEG or POEBA) is taken into a suitably dry solvent, such as THF. If further drying is necessary, the mixture can be dried over molecular sieves (4 Å) and filtered. The mixture is contacted with acrylyl chloride in the presence of TEA and DMAP. In some embodiments, an excess of the acylating agent is used. The reaction can be performed at 0° C., 5° C., 10° C., 20° C., room temperature, 30° C., 40° C., 50° C. or at reflux. In some embodiments, the components are combined at a lower temperature, such as 0° C., and then warmed to a higher temperature, such as room temperature. The reaction can proceed for 0.5 h, 1 h, 2 h, 3 h, 4 h, 6 h, 8 h, 10 h, 12 h, 16 h, 20 h, 24 h, overnight, or for more than a day. In some embodiments, one or more operations are performed in an inert atmosphere, such as nitrogen or argon. Upon completion, the product is obtained by any isolation technique known in the art, for example, extraction, or chromatography.

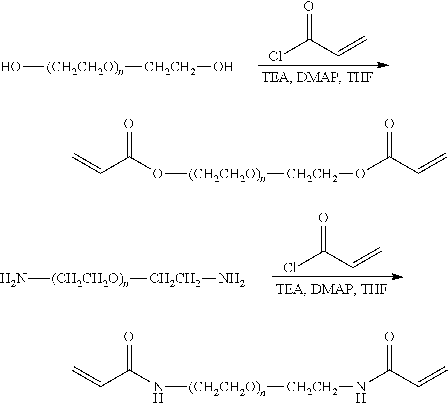

In another representative example, the acylating agent is 3-(N-maleimidyl)propionyl chloride. The experiment is performed as above.

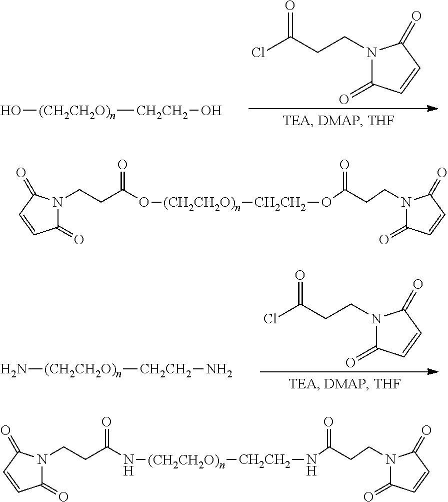

In another representative example, the acylating agent is maleic acid chloride. The experiment is performed as above.

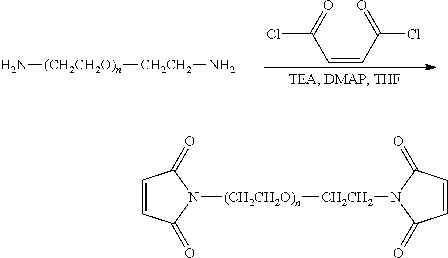

One of skill in the art recognizes that a large scope of compounds can be prepared by the synthetic techniques illustrated in these examples.

In some embodiments. PEG or POEBA is contacted with a mixture of acylating agents to provide a mixture of acylated products. Using a large number of acylating agents in such a procedure provides a library of products, which can be carried forward in a combinatorial synthesis of compounds of the invention.

The schemes illustrated above provide bifunctional electrophiles, which are capable of reacting with the sulfhydryl group of the side chain of a cysteine residue of a peptide or peptide analog such as enfuvirtide or exenatide. Compounds of Formula (I) can be prepared by contacting bifunctional electrophiles of Formula (II):

RG-(CH₂CH₂O)ₙ—CH₂CH₂-RG (II),

with peptides described herein.

In a representative example, a bifunctional electrophile is taken into a suitable solvent. Non-limiting examples of suitable solvents include methyl acetate (MeOAc), ethyl acetate (EtOAc), methanol (MeOH), ethanol (EtOH), isopropanol (iPrOH), dichloromethane (DCM), chloroform (CHCl₃), carbon tetrachloride (CCl₄), dimethylsulfoxide (DMSO), dimethylformamide (DMF), N-methylpyrrolidinone (NMP), acetonitrile (MeCN), and dimethylacetamide. A peptide described herein is contacted to the bifunctional electrophile. The peptide is optionally provided as a mixture in a solvent. The solvent can be the same as or different from the solvent into which the bifunctional electrophile was taken. In some embodiments, a base is optionally added to the reaction mixture. Non-limiting examples of suitable bases for the reaction include lithium hydroxide, sodium hydroxide, potassium hydroxide, cesium hydroxide, lithium methoxide, sodium methoxide, potassium methoxide, cesium methoxide, lithium ethoxide, sodium ethoxide, potassium ethoxide, cesium ethoxide, TEA, DIEA, 2,6-lutidine, and 2,6-di-t-butyl-4-methylpyridine. In some embodiments, a Lewis acid is optionally added to the mixture. Non-limiting examples of suitable Lewis acids include silica, alumina, trimethyl borate. The reaction can be performed at 0° C., 5° C., 10° C., 20° C., room temperature, 30° C., 40° C., 50° C., or at reflux. In some embodiments, the components are combined at a lower temperature, such as 0° C., and then warmed to a higher temperature, such as room temperature. The reaction is monitored by any technique known in the art, for example, thin layer chromatography (TLC), mass spectrometry (MS), or high performance liquid chromatography (HPLC). The reaction can proceed for 0.5 h, 1 h, 2 h, 3, h, 4 h, 6 h, 8 h, 10 h, 12 h, 16 h, 20 h, 24 h, overnight, or for more than a day. In some embodiments, one or more operations are performed in an inert atmosphere, such as nitrogen or argon. Upon completion, the product is obtained by any isolation technique known in the art, for example, extraction, or chromatography.

In a representative example, the bifunctional electrophile is bis(maleimide)PEG, and the peptide is the peptide of SEQ ID NO.: 38, and the reaction takes place in DCM without a base.

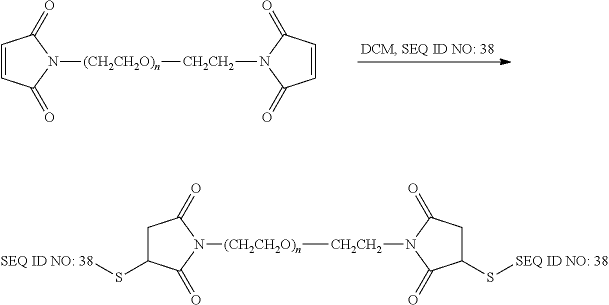

In some embodiments, a mixture of peptides is contacted to the bifunctional electrophile or to a mixture of several bifunctional electrophiles to provide a library of compounds of the invention.

Compounds of Formula (IV) can be prepared by an analogous method. A trifunctional electrophile is prepared by a process wherein the triol is contacted with a bifunctional electrophile in a solvent, optionally in the presence of a base. Non-limiting examples of suitable solvents include tetrahydrofuran (THF), ether (Et₂O), glyme, diglyme, tetraglyme, dichloromethane (DCM), chloroform (CHCl₃), carbon tetrachloride (CCl₄), acetonitrile (MeCN), methyl acetate (MeOAc), ethyl acetate (EtOAc), dimethylsulfoxide (DMSO), dimethylformamide (DMF), N-methylpyrrolidinone (NMP), and dimethylacetamide. Non-limiting examples of suitable bases include triethylamine (TEA), diisopropylethylamine (DIEA), pyridine, 2,6-lutidine, 2,6-di-t-butyl-4-methylpyridine, lithium carbonate, sodium carbonate, potassium carbonate, cesium carbonate, lithium bicarbonate, sodium bicarbonate, potassium bicarbonate, and cesium bicarbonate. In some embodiments, an excess of the bifunctional electrophile is used to prevent or lessen either cyclization or the attack of two equivalents of triol on the same bifunctional electrophile. In some embodiments, a highly-concentrated reaction mixture is used to promote intermolecular reactions and to prevent or lessen cyclization. In some embodiments, slow addition of the triol to a large excess of a bifunctional nucleophile is used to prevent or lessen the attack of two equivalents of triol on the same bifunctional electrophile. In some embodiments, a large excess of the bifunctional electrophile is used. In some embodiments, the molar excess of the bifunctional electrophile is 4, 5, 6, 7, 8, 9, 10, 15, 20, 30, 40, 50, 60, 70, 80, 90, or 100 equivalents of bifunctional electrophile for every one equivalent of triol.

In a representative example, a bifunctional electrophile, bis(maleimide)PEG, is contacted with the triol in THF in the presence of potassium carbonate (K₂CO₃) to provide a trifunctional electrophile.

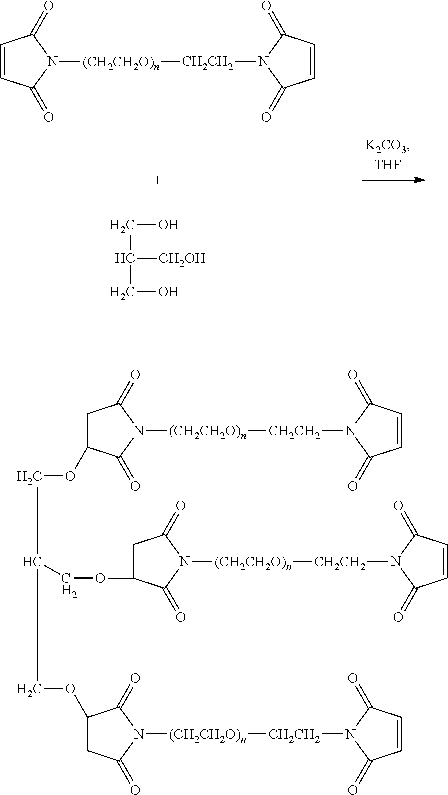

In some embodiments, a mixture of bifunctional electrophiles is contacted with the triol to provide a library of trifunctional electrophile's, which can be carried forward in the synthesis of compounds of the invention by a combinatorial approach.

A representative procedure for the introduction of the peptide groups is similar to the procedure described above for the introduction of peptide groups. The trifunctional electrophile illustrated above is contacted with the peptide of SEQ ID NO.: 38, and the reaction takes place in DCM without a base.

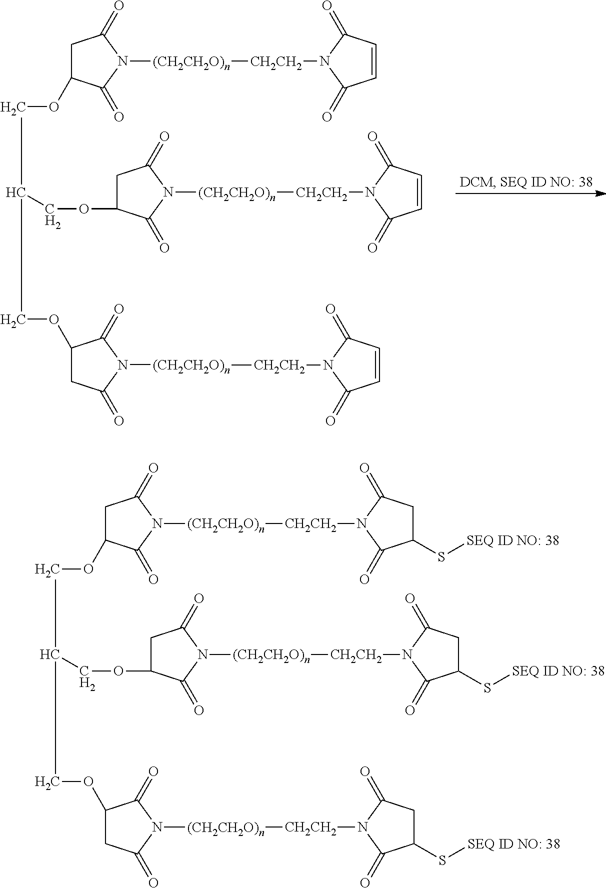

In some embodiments, a mixture of peptides is contacted to the trifunctional electrophile or to a mixture of several trifunctional electrophiles to provide a library of compounds of the invention.

Methods of Making Peptides

Synthesis of the peptides can be accomplished by any means known to one of skill in the art, including solid-phase peptide synthesis, solution-phase peptide synthesis, and automated peptide synthesis on a peptide synthesizer. Peptides can also be prepared physiologically by providing a microorganism with a vector suitable for the preparation of a peptide of the invention. Peptides can also be prepared from a broth containing transcriptional and translational machinery suitable for the synthesis of the peptides from a suitable nucleic acid molecule.

In addition, the invention is directed to a method of producing enfuvirtide and/or exenatide which may be used therapeutically or may be used as a starting material to produce a compound as described herein.

As an example, enfuvirtide may be produced with a vector comprising at least one promoter and sequences encoding each of the following: a) an affinity tag, b) an inclusion body targeting tag, c) a chemically cleavable tag, and d) SEQ ID NO: 1 (enfuvirtide) or an enfuvirtide analog having at least one difference in amino acid sequence from enfuvirtide. Such difference may be in having more than 36 amino acids, more than 36 amino acids with one or more substitutions, exactly 36 amino acids with one or more substitutions, deletions of one or more amino acids, or deletions of amino acids with substitutions of the remaining amino acids compared to enfuvirtide. Moreover, the enfuvirtide analog may contain at least one cysteine, at least one modified natural amino acid, at least one modified or unmodified non-natural amino acid, at least one amino acid analog, or combinations thereof. In various embodiments, an enfuvirtide analog has at least 50%, at least 60%, at least 70%, at least 80%, at least 90%, or more bioactivity compared to enfuvirtide. As a non-limiting example, the affinity tag may be poly-histidine, poly-lysine, poly-aspartic acid, or poly-glutamic acid. A non-limiting example of an inclusion body targeting tag is a ketoisomerase protein or fragment thereof. In various embodiments, the chemically cleavable tag is Trp. His-Met, or Pro-Met.

As another example, exenatide may be produced with a vector comprising at least one promoter and sequences encoding each of the following: a) an affinity tag, b) an inclusion body targeting tag, c) a chemically cleavable tag, and d) SEQ ID NO:78 (exenatide) or an exenatide analog having at least one difference in amino acid sequence from exenatide. Such difference may be in having more than 39 amino acids, more than 39 amino acids with one or more substitutions, exactly 39 amino acids with one or more substitutions, deletions of one or more amino acids, or deletions of amino acids with substitutions of the remaining amino acids compared to exenatide. Moreover, the exenatide analog may contain at least one cysteine, at least one modified natural amino acid, at least one modified or unmodified non-natural amino acid, at least one amino acid analog, or combinations thereof. In various embodiments, an exenatide analog has at least 50%, at least 60%, at least 70%, at least 80%, at least 90%, or more bioactivity compared to exenatide. As a non-limiting example, the affinity tag may be poly-histidine, poly-lysine, poly-aspartic acid, or poly-glutamic acid. A non-limiting example of an inclusion body targeting tag is a ketoisomerase protein or fragment thereof. In various embodiments, the chemically cleavable tag is Trp, His-Met, or Pro-Met.

In certain embodiments directed to a method of producing enfuvirtide or an enfuvirtide analog, the method according to the invention may comprise: i) obtaining a vector as described above; ii) transforming said vector into a host cell; iii) incubating the host cell for a time sufficient for production of peptides from said vector; iv) isolating enfuvirtide or an enfuvirtide analog from said incubating step. The method may also comprise the steps of v) separating inclusion bodies from the host cell; vi) extracting said inclusion bodies; vii) adding the extract to an affinity material; viii) washing the affinity material; ix) adding a chemical cleavage agent to the affinity material; x) separating cleaved product from the affinity material; and xi) optionally performing chemical modification of the amino and carboxy terminus of the cleaved product or chemical modification of one or more amino acid side chains.

In certain embodiments directed to a method of producing exenatide or an exenatide analog, the method according to the invention may comprise: i) obtaining a vector as described above; ii) transforming said vector into a host cell; iii) incubating the host cell for a time sufficient for production of peptides from said vector; iv) isolating exenatide or an exenatide analog from said incubating step. The method may also comprise the steps of v) separating inclusion bodies from the host cell; vi) extracting said inclusion bodies; vii) adding the extract to an affinity material; viii) washing the affinity material; ix) adding a chemical cleavage agent to the affinity material; x) separating cleaved product from the affinity material; and xi) optionally performing chemical modification of the amino and carboxy terminus of the cleaved product or chemical modification of one or more amino acid side chains.

Pharmaceutical Compositions, Administration, and Dosage

Compounds of the invention can be formulated into a variety of pharmaceutical compositions for use in therapy. The pharmaceutical composition facilitates administration of the compound to an organism. Pharmaceutical compositions can comprise other components in addition to active agents, such as carriers, stabilizers, diluents, dispersing agents, suspending agents, thickening agents, excipients, buffering agents, salts, surfactants, carbohydrates, anti-microbial agents, antioxidants, BSA, cosmotropic agents, and/or other peptide/protein stabilizing agents. A non-limiting list of protein stabilizing agents may include sucrose, trehalose, glycerol, betaine, amino acids, and trimethylamine oxide. Additionally, protein or peptide stabilizing agents may include polyols, sugars, amino acids and amino acid analogs. Some non-limiting examples include erythritol, sorbitol, glycerol, fructose, trehalose, proline, beta-alanine, taurine and glycine betaine. See Jeruzalmi & Steitz, J. Mol. Biol. 274: 748-756 (1997).

Buffering agents are advantageously present in disaggregating and/or refolding mixtures to maintain a desired pH value or pH range. Inorganic buffer systems (phosphate, carbonate, among others) and organic buffer systems (citrate, Tris, MOPS, MES, HEPES, among others) are known to the art.

Pharmaceutical composition containing compounds of the invention can be administered in therapeutically effective amounts as pharmaceutical compositions by any form and route known in the art including, but not limited to: subcutaneous, intravenous, intramuscular, transcutaneous, oral, aural, rectal, parenteral, ophthalmic, pulmonary, transdermal, vaginal, nasal, and topical administration. A pharmaceutical composition of the invention can be administered orally, for example, as a tablet or a capsule, or by injection, for example, intravenously, intramuscularly, or subcutaneously. In some embodiments, the composition is in the form of a powder for combination with water, a solution, a suspension, an oil, a tablet, or a capsule. In one embodiment, a compound of the invention is formulated as a powder for combination with sterile water for injection. In some embodiments, the administering is subcutaneous, topical, intraaural, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral.

For oral administration, compounds of the invention can be formulated as tablets, powders, pills, dragees, capsules, liquids, gels, syrups, elixirs, slurries, suspensions and solutions. Solid pharmaceutical compositions can be formulated with suitable coatings, additives, binders, flavoring agents, etc. Non-limiting examples include sugars, starch, gum arabic, lubricants such as talc and magnesium stearate, polyvinylpyrrolidone, carbopol gel, polyethylene glycol, titanium dioxide, lacquers, stabilizers, and suitable organic solvents or solvent mixtures.

In some embodiments, the administering is by subcutaneous injection.

Pharmaceutical compositions for injection or infusion can be provided as sterile suspensions, solutions or emulsions in oily or aqueous vehicles, and can contain formulatory agents such as suspending, stabilizing and/or dispersing agents. Non-limiting examples of suitable solvents or vehicles include fatty oils such as sesame oil, or synthetic fatty acid esters, such as ethyl oleate or triglycerides, or liposomes. Aqueous suspensions can contain thickeners, such as sodium carboxymethyl cellulose, sorbitol, or dextran or solubilizers.

Administrations can occur twice a day, daily, every other day, once every two days, once every three days, once every four days, once every five days, once every six days, three times a week, twice a week, weekly, three times a month, twice monthly, monthly, once every two months, or at the instruction of a physician. Compounds of the invention can provide a therapeutic effect with fewer administrations or less frequent administrations than an unmodified peptide (for example enfuvirtide, or exenatide). This phenomenon is the result of one or more properties of a compound of the invention that is superior to the analogous properties of enfuvirtide. Non-limiting examples of such properties include binding affinity, solubility, metabolic stability, physiological half-life, clearance, and distribution.

Doses of compounds of the invention can vary based on the identity, physiological properties, efficacy, and molecular weight of the compound. In some embodiments, a dose of a compound of the invention has about the same therapeutic effect as a 90 mg dose of enfuvirtide. In some embodiments, a dose of a compound of the invention provides a therapeutic effect that is greater than that of a 90 mg dose of enfuvirtide. The therapeutic effect can be greater for any reason described herein.

Doses of compounds of the invention can vary based on the identity, physiological properties, efficacy, and molecular weight of the compound. In some embodiments, a dose of a compound of the invention has about the same therapeutic effect as a 5 mcg dose of exenatide. In some embodiments, a dose of a compound of the invention provides a therapeutic effect that is greater than that of a 5 mcg dose of exenatide. The therapeutic effect can be greater for any reason described herein.

In some embodiments, a dose comprises a therapeutically-effective amount of enfuvirtide. In some embodiments, a dose comprises an amount of enfuvirtide that is therapeutically-effective for the treatment of HIV and/or AIDS. In some embodiments, a dose comprises an amount of enfuvirtide that is therapeutically-effective for the treatment of HIV-1. In some embodiments, a dose contains from 1 to 1,000 mg of a compound of the invention. In some embodiments, a dose contains from about 1 to about 1.000 mg of a compound of the invention. In some embodiments, a dose contains from 10 to 500 mg of a compound of the invention. In some embodiments, a dose contains from about 10 to about 500 mg of a compound of the invention. In some embodiments, a dose contains from 25 to 250 mg of a compound of the invention. In some embodiments, a dose contains from about 25 to about 250 mg of a compound of the invention. In some embodiments, a dose contains from 50 to 150 mg of a compound of the invention. In some embodiments, a dose contains from about 50 to about 150 mg of a compound of the invention. In some embodiments, a dose contains 100 mg of a compound of the invention. In some embodiments, a dose contains about 100 mg of a compound of the invention. In some embodiments, a dose contains 90 mg of a compound of the invention. In some embodiments, a dose contains about 90 mg of a compound of the invention. In some embodiments, a dose contains 10, 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, 900, 950, 1,000, 1,100, 1,200, 1,300, 1,400, 1,500, 1,600, 1,700, 1,800, 1,900, or 2,000 mg of a compound of the present invention.

In some embodiments, a dose comprises a therapeutically-effective amount of exenatide. In some embodiments, a dose comprises an amount of exenatide that is therapeutically-effective for the treatment of diabetes mellitus type 2. In some embodiments, a dose contains from 0.01 to 1,000 mcg of a compound of the invention. In some embodiments, a dose contains from about 0.01 to about 1,000 mcg of a compound of the invention. In some embodiments, a dose contains from 0.1 to 100 mcg of a compound of the invention. In some embodiments, a dose contains from about 0.1 to about 100 mcg of a compound of the invention. In some embodiments, a dose contains from 1 to 10 mcg of a compound of the invention. In some embodiments, a dose contains from about 1 to about 10 mcg of a compound of the invention. In some embodiments, a dose contains from 2 to 7 mcg of a compound of the invention. In some embodiments, a dose contains from about 2 to about 7 mcg of a compound of the invention. In some embodiments, a dose contains 5 mcg of a compound of the invention. In some embodiments, a dose contains about 5 mcg of a compound of the invention. In some embodiments, a dose contains 10 mcg of a compound of the invention. In some embodiments, a dose contains about 10 mg of a compound of the invention. In some embodiments, a dose contains 0.1, 1, 10, 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, 900, 950, or 1,000, mcg of a compound of the present invention.

In some embodiments, the amount of enfuvirtide-type compound is effective to provide about the same level of therapy as 90 mg of enfuvirtide. In some embodiments, the amount is effective to provide a level of therapy greater than the level of therapy provided by 90 mg of enfuvirtide. In some embodiments, the administered amount of enfuvirtide-type compound is about 10 to about 2,000 mg. In some embodiments, the amount is about 100 to about 1,000 mg. In some embodiments, the amount is about 250 to about 500 mg.

In some embodiments, the amount of exenatide-type compound is effective to provide about the same level of therapy as 5 mcg of exenatide. In some embodiments, the amount is effective to provide a level of therapy greater than the level of therapy provided by 5 mcg of exenatide. In some embodiments, the amount is about 0.01 to about 1,000 mcg. In some embodiments, the amount is about 0.1 to about 100 mcg. In some embodiments, the amount is about 1 to about 10 mcg.

In some embodiments, the administering takes place from 1 to 10 times daily, or twice daily, or weekly, semi-monthly, or monthly.

Therapeutic Methods

In some embodiments, compounds of the present invention and pharmaceutical compositions comprising the same are useful for providing therapy to subjects suffering from diseases or disorders that can be treated with peptides.

In some embodiments, compounds of the present invention and pharmaceutical compositions comprising the same are useful for providing therapy to subjects suffering from human immunodeficiency virus (HIV) and/or acquired immunodeficiency syndrome (AIDS). In some embodiments, a subject carries HIV-1. In some embodiments, a subject is in need or want of therapy for HIV and/or AIDS. In some embodiments, a compound of the invention interferes with the ability of an HIV virus to fuse with the surface of a target cell within the subject. In some embodiments, a compound of the invention interferes with the ability of an HIV virus to enter a target cell within the subject. In some embodiments, a compound of the invention slows, encumbers, or interferes with the proliferation, advancement, spread, or worsening of HIV and/or AIDS. In some embodiments, a compound of the invention improves the condition or quality of life of a subject suffering from HIV or AIDS.

In some embodiments, the invention provides the use of a compound in preparing a medicament for treating HIV and/or AIDS in a subject.

In some embodiments, compounds of the present invention and pharmaceutical compositions comprising the same are useful for providing therapy to subjects suffering from diabetes mellitus type 2. In some embodiments, compounds of the present invention and pharmaceutical compositions comprising the same are useful for providing therapy to subjects suffering from fatty liver or who are overweight. In some embodiments, a subject is in need or want of therapy for diabetes mellitus type 2. In some embodiments, a compound of the invention augments pancreas response (i.e., increases insulin secretion) in response to eating meals. In some embodiments, a compound of the invention suppresses pancreatic release of glucagon in response to eating. In some embodiments, a compound of the invention helps slow down gastric emptying and thus decreases the rate at which meal-derived glucose appears in the bloodstream. In some embodiments, a compound of the invention reduces liver fat content. In some embodiments, a compound of the invention improves the condition or quality of life of a subject suffering from diabetes mellitus type 2.

In some embodiments, the invention provides the use of a compound in preparing a medicament for treating diabetes mellitus type 2 in a subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

### Example 1

### Synthesis of a Compound of the Formula

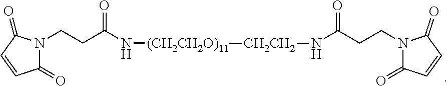

To a solution of POEBA [H₂N—(CH₂CH₂O)₁₁—CH₂CH₂—NH₂, 544.67 mg, 1 mmol] in DCM (50 mL) is added 3-(N-maleimidyl)propionyl chloride (2.1 mmol) in DCM (50 mL) at 0° C. under an argon atmosphere with stirring. Triethylamine (5 mmol) is added slowly via syringe, followed by DMAP (0.1 mmol). The mixture is allowed to come to room temperature slowly, and stirring is continued for 8 hours, or until the POEBA is no longer visible by TLC, HPLC, or MS. The mixture is diluted and washed with 1 N HCl (3×50 mL), then washed with saturated aqueous NaHCO₃ (3×50 mL), water (3×50 mL), and saturated aqueous sodium chloride (3×50 mL). The organic phase is dried over Na₂SO₄, filtered, and concentrated. Chromatography through a silica gel column provides the compound, which is analyzed by NMR, IR, MS, and HPLC.

### Example 2

### Synthesis of a Compound of the Formula

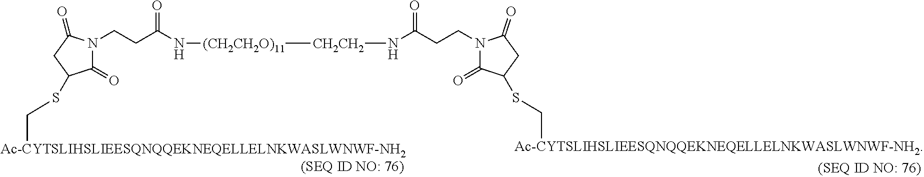

To a solution of the product of EXAMPLE 1 (1 mmol) in an aqueous buffer (100 mL) is added the peptide of SEQ ID NO.: 76 (2.1 mmol, 9.66 g) in aqueous buffer (300 mL) at room temperature and pH between 6-10. Prior to addition, the peptide is exposed to a 5-molar excess of dithiothreitol for 30 minutes to reduce and activate the sulfur of the peptide. The mixture is stirred for 24 hours under an argon atmosphere, or until the product of EXAMPLE 1 is no longer visible by TLC, HPLC, or MS. The reaction mixture is concentrated and the product is separated from the excess, unreacted peptide of SEQ ID NO.: 76 by HPLC. The product is analyzed by HPLC and MS.

### Example 3

### Synthesis of a Compound of the Formula

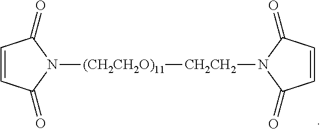

To a solution of POEBA [H₂N—(CH₂CH₂O)₁₁—CH₂CH₂—NH₂, 544.67 mg, 1 mmol] in DCM (50 mL) is added malonyl chloride (2.1 mmol) in DCM (50 mL) at 0° C. under an argon atmosphere with stirring. Triethylamine (10 mmol) is added slowly via syringe, followed by DMAP (0.1 mmol). The mixture is allowed to come to room temperature slowly, and stirring is continued for 24 hours, or until the POEBA is no longer visible by TLC. HPLC, or MS. The mixture is diluted and washed with 1 N HCl (3×50 mL), then washed with saturated aqueous NaHCO₃ (3×50 mL), water (3×50 mL), and saturated aqueous sodium chloride (3×50 mL). The organic phase is dried over Na₂SO₄, filtered, and concentrated to an oil. Chromatography through a silica gel column provides the compound, which is analyzed by NMR, IR, MS, and HPLC.

### Example 4

### Synthesis of a Compound of the Formula

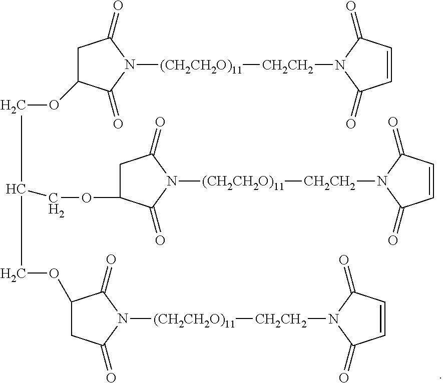

To a mixture of the product of EXAMPLE 3 (9 mmol) and K₂CO₃ (5 mmol) in THF (200 mL) is added a solution of 2-hydroxymethyl-1,3-propanediol (1 mmol) in THF (20 mL) dropwise over a period of 1 hour. The mixture is stirred for 8 hours, or until the triol is no longer visible by TLC, HPLC, or MS. The mixture is diluted with water (100 mL), the phases are separated, and the aqueous phase is extracted with EtOAc (3×50 mL). The organics are combined and washed with water (3×50 mL), saturated aqueous sodium chloride (3×50 mL). The organic phase is dried over Na₂SO₄, filtered, and concentrated to an oil. Chromatography through a silica gel column provides the compound, which is analyzed by NMR, IR, MS, and HPLC.

### Example 5

### Synthesis of a Compound of the Formula

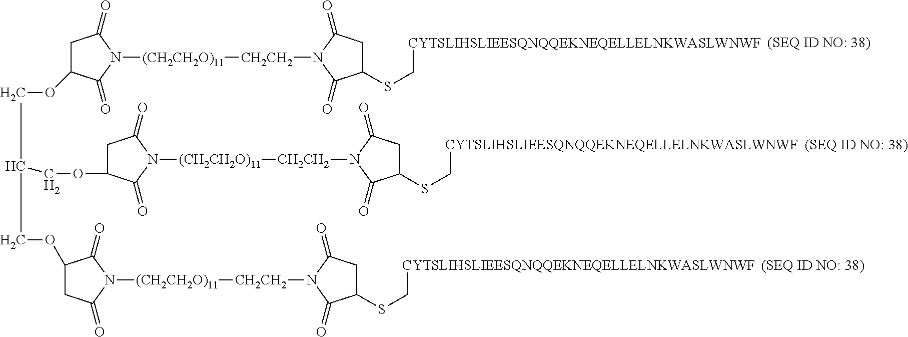

To a solution of the product of EXAMPLE 4 (1 mmol) in DCM (200 mL) is added the peptide of SEQ ID NO.: 38 (3.3 mmol, 15.18 g) in DCM (500 mL) at room temperature. The mixture is stirred for 24 hours under an argon atmosphere, or until the product of EXAMPLE 4 is no longer visible by TLC, HPLC, or MS. The reaction mixture is concentrated and the product is separated from the excess, unreacted peptide of SEQ ID NO.: 38 by HPLC. The product is analyzed by HPLC and MS.

### Example 6

### Expression and Modification of Enfuvirtide and Analogs

Wild type enfuvirtide and two analogs (also referred to as enfuvirtide muteins) are expressed via a bacterial expression system. The muteins contain added cysteine residues. The expressed polypeptides are fusion polypeptides including the n-terminal fragment of a ketosteroid isomerase as an inclusion body inducing tag. The expression vectors also contain an affinity tag (polyhistidine). Alternatively, PCR mutagenesis experiments are performed to add a cysteine residue to the N- or C-terminus of enfuvirtide for the eventual covalent linkage to a homobifunctional PEG linker. Alternatively, commercially available protein expression systems (such as Impact from New England Biolabs) are used to generate a peptide with an N-terminal cysteine.

Expressed fusion polypeptides are isolated by affinity chromatography (IMAC) under denaturing conditions, cleaved, and further purified. In the absence of any methionine residues, cyanogen bromide may be used for cleavage.

Polypeptides are optionally analyzed by RP-HPLC analysis. SDS-PAGE, mass spectral analysis, N-terminal analysis and/or peptide mapping. Commercial available enfuvirtide and recombinantly-produced native enfuvirtide are used as controls for the assays (with the assumption that the added cysteine will alter MW, retention times, etc.). Enfuvirtide is N-terminally acetylated and C-terminally amidated. If desired, muteins are enzymatically treated to introduce an amide group to the C-terminal carboxylic acid or chemically acetylated at an N-terminal amino acid.

Prior to addition of PEG, the cysteine muteins are partially reduced with dithiothreitol (DTT) in order to expose the free cysteine for PEGylation and to allow the PEGylation reaction to proceed efficiently. Typically a 5-fold molar excess of DTT for 30 min is sufficient. Excess DTT is removed by size exclusion chromatography or dialysis.

The reduced peptide is reacted with various concentrations of 10 kDa PEG-maleimide (PEG: protein molar ratios of around 1:2 for the dimer and 1:1 for the monomer). A variety of monofunctional and homobifunctional PEG reagents are available commercially (NOF, Japan). PEGylation of the peptide is monitored by a molecular weight shift using SDS-PAGE. Solvents or detergents are optionally added to the reaction to maintain solubility. Dimeric PEGylated peptide is purified from any mono-PEGylated and unPEGylated peptide by hydrophobic interaction or ion exchange chromatography. Concentrations of purified PEGylated peptides are measured using UV spectroscopy or by the Bradford protein assay since the PEG does not interfere with dye binding to a polypeptide. Additional post-PEGylation assays, including SEC-HPLC analysis, SDS-PAGE, mass spectral analysis, N-terminal analysis, peptide mapping or endotoxin determination, are performed.

The location of PEG attachment is analyzed by proteolytic digestion of the peptide, purification of the PEG peptide, and sequencing of the amino acid. The PEG-coupled amino acid appears as a blank during sequencing. The secondary structures of enfuvirtide, the enfuvirtide muteins and PEGylated enfuvirtide are evaluated using circular dichroism. PEG does not interfere with this assay; therefore this assay is a sensitive analytical technique for verifying conformation.

### Example 7A

### Selecting Active Compounds from a Library of Compounds of the Invention

A library of compounds is prepared using the protocols of EXAMPLES 1-6 with a variety of PEG and POEBA moieties, acylating agents, and peptides. The library is taken into DMSO and diluted in physiological saline. The resultant mixture is eluted through an affinity column containing gp41 supported on a resin. The column is flushed with saline to remove the low-affinity compounds. The column is then eluted with an aqueous suspension of enfuvirtide. Elution is reiterated until HPLC analysis of the eluent shows the presence of enfuvirtide only. The eluent fractions are resolved by HPLC to separate the compounds of the invention from the excess enfuvirtide. The high-affinity compounds identified by this protocol are analyzed and characterized by HPLC and MS.

### Example 7B

### Selecting Active Compounds from a Library of Compounds of the Invention

A library of compounds is prepared using the protocols of EXAMPLES 1-6 with a variety of linking moieties such as PEG and POEBA moieties, acylating agents, and peptides. The library is taken into DMSO and diluted in physiological saline. The resultant mixture is eluted through an affinity column containing anti-thrombin supported on a resin. The column is flushed with saline to remove the low-affinity compounds. The column is then eluted with an aqueous suspension of enfuvirtide or other agent for which affinity is being compared. Elution is reiterated until HPLC analysis of the eluent shows the presence of enfuvirtide or other agent only. The eluent fractions are resolved by HPLC to separate the compounds of the invention from the excess enfuvirtide or other agent. The compounds identified by this protocol are analyzed and characterized by HPLC and MS.

### Example 8

### Bioassays

The bioactivity of enfuvirtide cysteine muteins and PEGylated enfuvirtide peptides is evaluated in a cell-cell syncytium-formation assay. The syncytial inhibition assay is run with HeLa-CD4-LTR-b-galactosidase cells (Buckheit et al., 1994). Briefly, the cell-cell fusion inhibition assay is performed in flat-bottom, 96-well microtiter plates. HeLa-CD4-LTR-β-galactosidase cells (5×103) are added to each well, and the cells are incubated with test compound for 1 h prior to the addition of 5×103 HL2/3 cells. The cells were incubated for an additional 48 h and fixed and stained with X-Gal. Blue syncytia are counted microscopically. The validity of this assay for anti-HIV substances which inhibit syncytium formation has been confirmed using various known HIV entry inhibitors (Bukheit et al., 1994)

Pharmacokinetic (PK) studies of PEGylated enfuvirtide peptides are performed to determine to what extent PEGylation lengthens the in vivo half-life of the peptide. A PK study compares wild type enfuvirtide, 10 kDa PEGylated enfuvirtide and a 10 kDA PEGylated enfuvirtide dimer. Three rats receive a subcutaneous bolus injection (4 mg/kg) of one of the test peptides. Circulating levels of the proteins are measured over the course of 96 hr. Blood samples are collected at 0, 0.5, 1.5, 4, 8, 24, 48, 72 and 96 hr following administration. Peptide levels are determined by liquid chromatography-tandem mass spec (LC-MS/MS) after trypsin digestion of the plasma samples as described by Huet et al. 2010. Alternatively, an ELISA assay is performed. The protocol may be repeated with PEG linkers of different sizes (10, 20, and 40 kDa) and with different routes of administration (intravenous and subcutaneous).

### Example 9

### Virus-Free Cell Fusion Assay

This assay is performed using either HeLa-CD4-LTR (β-gal or U373-MAGI (Multinuclear Activation of a Galactosidase Indicator) cells expressing CD4 constitutively and β-galactosidase under the control of the HIV-1 LTR promoter; the U373-MAGI-CXCR4 (expressing in addition the CXCR4 gene); or the U373-MAGI-CCR5 (expressing in addition the CCR5 gene). Cells constitutively expressing the HIV-1 tat and GP160 (called HL160tat cells) are used because they express viral proteins that allow fusion with the HeLa or U373 cells. The tat protein switches on the LTR-driven β-galactosidase gene expression if the fusion occurs.

HeLa or U373 and HL160tat cells are co-cultivated in DMEM+2% FBS and incubated at 37° C. in 5% CO₂ in presence of ⅓ serial concentrations (ranging from 0.0045 to 10 μg/ml) of the compounds of the present invention for 24-48 hours.

The cells are fixed and the β-galactosidase reporter gene is detected with the X-gal substrate in case of fusion. The number of syncytia is counted and the IC₅₀ value is defined as the dilution that resulted in a 50% reduction of the syncytia formation.

In a variant of the above assay, testing is performed using U373 MAGI CXCR4 cells (CD4/CXCR4 expressing cells) and CHO-Wild Type cells (wild type HIV envelope protein-expressing cells). Both cells (250,000 cells each) are co-cultivated in EMEM+10% FBS and incubated overnight at 37° C. in 5% CO₂ in the presence of ⅓ serial concentrations (ranging from 0.4 to 300 nM) of the compounds of the present invention. The cells are then fixed using FIX-RAL 555 and syncytia are detected after cell surface staining with EOSINE-RAL 555 and BLEU-RAL 555. The number of syncytia is counted and the IC₅₀ value determined using Graphpad Prism Software.

Anti-HIV-1 Activity on MT-4 Cells

MT-4 cells are seeded in the presence of a compound of the invention and diluted with a composition containing HIV-1. Cytopathic effects induced by the virus are checked regularly by microscopy. After 4 days of infection, the cell viability is assessed spectrophotometrically using the MTT assay. The median inhibitory concentration (IC₅₀) is calculated from each dose-response curve.

The ability of the compounds of the present invention to decrease the cytopathic effect induced by HIV-1 on MT-4 cells is then observed.

UT7 Cell Proliferation Assay

The in vitro proliferative activity is assessed in human UT7 (acute myeloid leukaemia) cells.

UT7 cells are washed in α-MEM and starved in α-MEM with added 2 mM L-glutamine and 5% FBS for 4 hours. Then, the UT7 cells are washed in α-MEM, counted and suspended at 2×10⁵ cells/ml in α-MEM+10% FBS+Penicillin/streptomycin 1%+2 mM L-glutamine. A stock solution of a compound of the present invention is diluted, and then further diluted by 1:2 serial dilutions in α-MEM+10% FBS+Penicillin/streptomycin 1%+2 mM L-glutamine. The UT7 cell suspension and a dilution of a compound ranging from 0 to 2.7×10⁻¹⁰ M are mixed 1/1 (v/v), plated, and incubated at 37° C. in 5% CO₂ for approximately 68 hours.

The cell proliferation assay is assessed by adding Seroctec's Alamarblue reagent (10 μL). After 4-5 hours of incubation, the optical density of the mixture is measured.

The ability of the compounds of the present invention to induce UT7 cell proliferation is then observed.

Clonogenic Assay

The biological activity of the compounds of the present invention is assessed by the BFU-E clonogenic assay, which is well known in the art. Peripheral blood mononuclear cells (PBMC) are plated in methylcellulose-based medium containing FBS, rhuIL3 and SCF. The concentration of the compounds of the present invention is varied from 0 to 5.48×10⁻¹⁰ 10 M.

The PBMC cells are plated in 35 mm Petri dishes and incubated in a fully-humidified atmosphere with 5% CO₂ at 37° C. for 14 days.

The ability of the compounds of the present invention to enhance erythroid colony formation is then observed.

IFN-α

In Vitro Anti-Proliferative Activity in Human Tumor Cell Line ACHN

The anti-proliferative activity of compounds of the present invention is assessed in human ACHN (renal adenocarcinoma) cells.

ACHN cells are washed in EMEM, counted and suspended at 4×10⁴ cells/mL in EMEM+4-10% FBS+Penicillin/streptomycin 1%+2 mM L-glutamine (complete medium). 50 μL of cell suspension (2,000 cells/well) are plated and allowed to adhere.

For each concentration tested (ranging from 0 to 20.4×10⁻¹⁰ M), the compounds of the present invention are diluted in EMEM+Penicillin/streptomycin 1%+2 mM L-glutamine at a two times concentration and 50 μL of the diluent is added to the corresponding well.

The ACHN cells are incubated at 37° C. in 5% CO₂ for approximately 90 hours. The cell proliferation assay is then assessed by adding 3-(4,5-dimethylthiazol-2-yl)-2,5-diphenyltetrazolium (MT). After 4-5 hours of incubation, the optical density of the mixture is measured.

The ability of the compounds of the present invention to reduce ACHN cell proliferation is then observed.

Increase of MHC Class 1 Molecules Expression in MOLT-4 Cells

The increase of MHC class 1 molecules expression is assessed in human MOLT-4 (acute lymphoblastic leukaemia) cells.

The MOLT-4 cells are washed in RPMI 1640 and starved in RPMI 1640 with added L-glutamine overnight. Then, the MOLT-4 cells were washed in RPMI 1640, counted and suspended at 3×10⁵ cells/mL in RPMI 1640+10% FBS+Penicillin/streptomycin 1%+2 mM L-glutamine (compete medium) in the presence of various concentrations of the compounds of the present invention ranging from 0 to 5.2×10⁻¹⁰ M.

The MOLT-4 cells are incubated at 37° C. in 5% CO₂ for 48 hours, then stained with an antihuman HLA A,B,C from Serotec (0.1 μg/10 cells) and washed. The expression of MHC class 1 is assessed by flow cytometry.

The ability of the compounds of the present invention to increase the expression of MHC class 1 is then observed.

Alternatively, the bioactivity of enfuvirtide cysteine muteins and PEGylated enfuvirtide peptides is evaluated in a cell-cell syncytium-formation assay. The syncytial inhibition assay is run with HeLa-CD4-LTR-β-galactosidase cells as described by Buckheit et al., 1994. Briefly, the cell-cell fusion inhibition assay is performed in flat-bottom, 96-well microtiter plates. HeLa-CD4-LTR-β-galactosidase cells (5×10′) are added to each well, and the cells are incubated with test compound for 1 h prior to the addition of 5×10³ HL2/3 cells. The cells are incubated for an additional 48 h and fixed and stained with X-Gal. Blue syncytia are counted microscopically.

### Example 10

### Additional Pharmacokinetic Observations

PK studies of wild type enfuvirtide, 10 kDa PEGylated enfuvirtide and a 10 kDA PEGylated enfuvirtide dimer are performed to determine to what extent PEGylation lengthens the in vivo half-life of the peptide.

A solution of a compound of the present invention is prepared in Phosphate Buffered Saline (pH 7.4). If necessary, the compound is first dissolved in DMSO, then diluted in saline. The solution is administered intravenously to a female Wistar rat, and the pharmacokinetic properties are investigated after a single administration using a LCMS procedure. The blood of the animal is sampled on citrate tubes over a period of several days after the administration. After administration of a single intravenous dose in rats, the AUC_{(0-∞)} is also measured, in μg·h/ml.

In an alternate test, three rats receive a subcutaneous bolus injection (4 mg/kg) of a test compound. Circulating levels are measured over the course of 96 hr. Blood samples are collected at 0, 0.5, 1.5, 4, 8, 24, 48, 72 and 96 hr following administration. Peptide levels are determined by liquid chromatogrphy-tandem mass spec (LC-MS/MS) after trypsin digestion of the plasma samples as described by Huet et al. 2010. The assay can be modified for detection of a PEGylated peptide or for fragments of enfuvirtide after tryptic digestion to remove the PEG moiety.

### Example 11

### Creation of Novel Exenatide-Like Proteins

Novel bivalent PEGylated exenatide constructs using the maleimide linker chemistry that has already been approved for use in patients are created. Without wishing to be bound by theory, it is believed that this approach offers tunable pharmacokinetics based on the size of the PEG. The bivalent PEGylated exenatide has a higher avidity for its cellular target since it has the ability to bind to two surface receptors thanks to the flexibility of the PEG linker. Bivalent binding is more stable than monovalent binding since both ligands will have to dissociate simultaneously for a bivalently bound molecule to detach from a surface.

A novel exenatide-like protein with enhanced in vive characteristics such as an increased circulating half-life and improved efficacy through site-specific chemical modification of the protein is created by using the published structural information to rationally design polyethylene glycol (PEG)-exenatide conjugates using cysteine-reactive PEGs. A new “free” cysteine is introduced using site-directed mutagenesis in a region of exenatide that is believed to be non-essential for biological activity. The “free” cysteine residue serves as the site for the covalent modification of the peptide using a thiol-reactive PEG. Without wishing to be found by theory, it is believed that this allows for the creation of a novel, fully active PEG-Cys-exenatide analog of defined structure and overcomes the problem of reduced bioactivity and heterogeneity when peptides are modified using a standard amine-reactive PEG. The exenatide analog may be produced using recombinant/synthetic chemistry hybrid technology which is capable of generating gram quantities of peptide per fermentation liter. Lastly, a bivalent exenatide analog which is constructed using a homo bifunctional PEG reagent that is able to bind two exenatide molecules resulting in a dumbbell-like configuration is evaluated. This compound is measured for a higher avidity since it has the ability to bind to two surface receptors simultaneously thanks to the flexibility of the PEG linker. One or more PEG-modified exenatide peptides are created with a greater bioactivity and a significantly longer half-life in vivo versus the parent compound. The PEGylated exenatide peptides also have improved stability, greater solubility, and reduced antigenicity. These improved physical and biological characteristics allow for the rapid validation of efficacy in both pre-clinical and clinical studies for the treatment of type 2 diabetes.

Phase I

Phase I studies evaluate the in vitro bioactivity of a C-terminally linked PEGylated homodimer. Native exenatide and monoPEGylated exenatide are also tested. A pharmacokinetic animal study to determine to what extent 10 kDa PEG linker enhances the stability and circulating half-life of exenatide in vivo is performed.

Step 1. Wild type exenatide and a cysteine mutein of exenatide are expressed in bacteria.

Wild type exenatide is cloned and expressed using a bacterial peptide expression system as are 2 exenatide muteins, each containing a new “added” cysteine residue.

During the Phase I studies, native exenatide (exendin-4) as a C-terminal fusion to a larger polypeptide (an n-terminal fragment of ketosteroid isomerase) that will drive the expression of the attached peptide into bacterial inclusion bodies is cloned and expressed. The expression vector also contains an N-terminal affinity tag (polyhistidine). This approach minimizes the toxicity during bacterial growth, simplifies the downstream processing and lowers the overall cost of peptide manufacturing.

PCR mutagenesis experiments are performed to add a cysteine residue to the C-terminus of exendin-4 for the eventual covalent linkage to the homobifunctional PEG linker. The intein mediated protein expression system (Impact™ from New England Biolabs) is also capable of generating a peptide with a C-terminal cysteine.

Step 2. Each peptide is purified to homogeneity and initial characterization studies are performed.

The fusion protein is removed and each peptide is purified to homogeneity. Initial characterization studies are performed. The fusion proteins are isolated by affinity chromatography (IMAC) under denaturing conditions, cleaved, and further purified. Process and analytical methods development are performed simultaneously to enhance overall yields and verify the purity of each peptide produced. Standard assays performed prior to PEGylation include RP-HPLC analysis, SDS-PAGE, mass spectrometry. N-terminal analysis and peptide mapping. Commercially available exenatide and recombinantly-produced native exenatide are used as controls for these assays (with the assumption that the added cysteine will cause some variability in terms of MW, retention times, etc.). Exenatide is C-terminally amidated to protect the peptide from proteolytic digestion. The presence of a PEG moiety has a similar effect.

Step 3. Exenatide muteins are PEGylated with maleimide and bimaleimide 10 kDa PEGs, and the PEGylated peptides are purified.

PEGylate the exenatide mutein with a cysteine-reactive maleimide-10 kDa and bimaleimide 10 kDa PEGs. Purify the PEGylated peptides. Perform additional characterization studies.

After prokaryotic expression, the cysteine muteins are partially reduced with dithiothreitol (DTT) in order to expose the free cysteine for PEGylation to allow the PEGylation reaction to proceed efficiently. Although the free cysteine is not involved in a disulfide bond, it is largely unreactive to cysteine-reactive PEGs unless this reduction step is performed. Typically a 5-fold molar excess of DTT for 30 min is sufficient. Wild type exenatide contains no native cysteines so there is no risk of reducing a native disulfide. Excess DTT will be removed by size exclusion chromatography or dialysis. Alternatively the reducing agent tris(2-carboxyethyl)phosphine (TCEP) can be used which will not require dialysis. The reduced peptide is reacted with various concentrations of 10 kDa PEG-maleimide (PEG: protein molar ratios of around 1:2 for the dimer and 1:1 for the monomer) to determine the optimum ratio. A variety of monofunctional and homobifunctional PEG reagents are available from NOF (Japan). PEGylation of the peptide is monitored by a molecular weight shift using SDS-PAGE. Conversion yields are greater than 80% based. The dimeric PEGylated peptide from any mono-PEGylated and unPEGylated peptide by hydrophobic interaction or ion exchange chromatography is purified. Concentrations of purified PEGylated peptides are measured using UV spectroscopy or by the Bradford protein assay. Additional analytical assays performed post-PEGylation include SEC-HPLC analysis, SDS-PAGE, mass spectral analysis, N-terminal analysis, peptide mapping and endotoxin determination.

Step 4. In vitro bioactivities of wild type exenatide, the exenatide mutein, and the PEGylated exenatide muteins are measured in a cell-based assay.

The bioactivity of exenatide cysteine mutein and the PEGylated exenatide peptides is evaluated in a GLP-1 receptor binding assay cell. Rat pancreatic epithelial cells (pancreatic (3-cell model: ATCC) are treated with 5 μM staurosporine (an apoptosis inducer) in the presence of 0, 10, 20 or 40 nM exenatide for 16, 24, or 48 hours respectively. Cell viability is evaluated using Cell Titer-Glo® (Promega).

Step 5. A small pharmacokinetic experiment in rats using subcutaneous administration to demonstrate an increased circulating half-life for the 10 kDa PEGylated exenatide peptides versus wild type exenatide is performed.

PK studies of PEGylated exenatide peptides are performed to determine to what extent PEGylation lengthens the in vivo half-life of the peptide. This PK study tests wild type exenatide, 10 kDa PEGylated exenatide and a 10 kDA PEGylated exenatide dimer. Three rats receive a subcutaneous bolus injection (4 mg/kg) of one of the test peptides. Circulating levels of the proteins are measured over the course of 96 hr. Blood samples are collected at 0, 0.5, 1.5, 4, 8, 24, 48, 72 and 96 hr following administration. Peptide levels are determined by a commercially available ELISA kit (R&D Systems). The assay is calibrated using PEGylated exenatide standards because the PEG moiety will lower the anti-exenatide antibody's response. PEGylation significantly extends the circulating half-life of both the monoPEGylated exenatide and PEG dimer relative to unPEGylated exenatide.

Phase II

Step 1. Mutagenesis studies for identification of the best PEGylated exenatide construct for pre-clinical development are performed. Also newer more potent sequence variants of exenatide are cloned and expressed.

The effects of cysteine substitutions and PEGylation at additional sites in exenatide to determine the effects of these modifications on the bioactivity of the peptide are analyzed. The usefulness of extending the size of exenatide by including additional amino acids on one or both ends is examined. These types of modifications do not adversely impact the complexity of the manufacturing process as would be the case with a chemically synthesized peptide. These extensions are used as alternative sites for the addition of a free cysteine. Lastly, newly reported more potent sequence variants of exenatide are investigated.

Step 2. Methods are developed and biochemical and structural characterization of those muteins and their PEGylated variants with wild type activity to verify purity, stability, site of PEGylation, etc, is performed.

Once PEGylated exenatide muteins with high in vitro biological activity are identified, we confirm that the PEG molecule is attached to the peptide at the proper site. This is accomplished by proteolytic digestion of the peptide, purification of the PEG peptide, followed by amino acid sequencing. The secondary structures of exenatide, the exenatide muteins and PEGylated exenatides are evaluated using circular dichroism. PEG does not interfere with this assay; therefore it provides a sensitive analytical technique for verifying conformation. Other analytical assays that were previously mentioned are qualified for sensitivity, accuracy, precision, and robustness.

Step 3. The process is optimized for the production of PEGylated exenatide.

The bacterial expression, chemical cleavage, purification, and PEGylation protocols are optimized, evaluating final yields, purities, and specific activities for scale up.

Step 4. Gram quantities of the most promising PEGylated exenatide modified with 10, 20, or 40 kDa PEGs are prepared.

Step 5. More extensive pharmacokinetic studies are performed.

During Phase II, the PK studies initiated during Phase I are extended. PEG molecules of different sizes (10, 20, and 40 kDa) and different routes of administration (intravenous and subcutaneous) are explored. Protocols described in Phase I are followed for the injections and for analyzing blood samples.

Step 6. The relative efficacies of the PEGylated exenatides versus the parent molecule in additional in vitro and in vivo models of diabetes are compared.

Six-week-old male C57BL/6 db/db mice are used for the acute antidiabetic activity tests, after being acclimatized for 1-week in an animal facility. Under nonfasting conditions with free access to water and food, mice are administered a single subcutaneous injection of exenatide or a PEGylated version of exenatide (15 nmol/kg, 200 μL, s.c., 6 mice per group). Blood glucose levels are then monitored using a glucometer and tail-tip blood samples (0, 0.5, 1, 2, 4, 6, 8, 12, 20, and 24 h after administration). Data is expressed as the means±SDs. The student's t-test is used throughout, and values of p<0.05 are considered statistically significant.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

TABLE 1

| SEQ ID | |
|---|---|
| NO. | Sequence |
| SEQ ID | YTSLIHSLIEESQNQQEKNEQELLELNKWASLWNWF |
| NO.: 1 | |
| SEQ ID | CTSLIHSLIEESQNQQEKNEQELLELNKWASLWNWF |
| NO.: 2 | |
| SEQ ID | YCSLIHSLIEESQNQQEKNEQELLELNKWASLWNWF |
| NO.: 3 | |
| SEQ ID | YTCLIHSLIEESQNQQEKNEQELLELNKWASLWNWF |
| NO.: 4 | |
| SEQ ID | YTSCIHSLIEESQNQQEKNEQELLELNKWASLWNWF |
| NO.: 5 | |
| SEQ ID | YTSLCHSLIEESQNQQEKNEQELLELNKWASLWNWF |
| NO.: 6 | |
| SEQ ID | YTSLICSLIEESQNQQEKNEQELLELNKWASLWNWF |
| NO.: 7 | |
| SEQ ID | YTSLIHCLIEESQNQQEKNEQELLELNKWASLWNWF |
| NO.: 8 | |
| SEQ ID | YTSLIHSCIEESQNQQEKNEQELLELNKWASLWNWF |
| NO.: 9 | |
| SEQ ID | YTSLIHSCEESQNQQEKNEQELLELNKWASLWNWF |
| NO.: 10 | |
| SEQ ID | YTSLIHSLICESQNQQEKNEQELLELNKWASLWNWF |
| NO.: 11 | |
| SEQ ID | YTSLIHSLIECSQNQQEKNEQELLELNKWASLWNWF |
| NO.: 12 | |
| SEQ ID | YTSLIHSLIEECQNQQEKNEQELLELNKWASLWNWF |
| NO.: 13 | |
| SEQ ID | YTSLIHSLIEESCNQQEKNEQELLELNKWASLWNWF |
| NO.: 14 | |
| SEQ ID | YTSLIHSLIEESQCQQEKNEQELLELNKWASLWNWF |
| NO.: 15 | |
| SEQ ID | YTSLIHSLIEESQNCQEKNEQELLELNKWASLWNWF |
| NO.: 16 | |
| SEQ ID | YTSLIHSLIEESQNQCEKNEQELLELNKWASLWNWF |
| NO.: 17 | |
| SEQ ID | YTSLIHSLIEESQNQQCKNEQELLELNKWASLWNWF |
| NO.: 18 | |
| SEQ ID | YTSLIHSLIEESQNQQECNEQELLELNKWASLWNWF |
| NO.: 19 | |
| SEQ ID | YTSLIHSLIEESQNQQEKCEQELLELNKWASLWNWF |
| NO.: 20 | |
| SEQ ID | YTSLIHSLIEESQNQQEKNCQELLELNKWASLWNWF |
| NO.: 21 | |
| SEQ ID | YTSLIHSLIEESQNQQEKNECELLELNKWASLWNWF |
| NO.: 22 | |
| SEQ ID | YTSLIHSLIEESQNQQEKNEQCLLELNKWASLWNWF |
| NO.: 23 | |
| SEQ ID | YTSLIHSLIEESQNQQEKNEQECLELNKWASLWNWF |
| NO.: 24 | |
| SEQ ID | YTSLIHSLIEESQNQQEKNEQELCELNKWASLWNWF |
| NO.: 25 | |
| SEQ ID | YTSLIHSLIEESQNQQEKNEQELLCLNKWASLWNWF |
| NO.: 26 | |
| SEQ ID | YTSLIHSLIEESQNQQEKNEQELLECNKWASLWNWF |
| NO.: 27 | |
| SEQ ID | YTSLIHSLIEESQNQQEKNEQELLELCKWASLWNWF |
| NO.: 28 | |
| SEQ ID | YTSLIHSLIEESQNQQEKNEQELLELNCWASLWNWF |
| NO.: 29 | |
| SEQ ID | YTSLIHSLIEESQNQQEKNEQELLELNKCASLWNWF |
| NO.: 30 | |
| SEQ ID | YTSLIHSLIEESQNQQEKNEQELLELNKWCSLWNWF |
| NO.: 31 | |
| SEQ ID | YTSLIHSLIEESQNQQEKNEQELLELNKWACLWNWF |
| NO.: 32 | |
| SEQ ID | YTSLIHSLIEESQNQQEKNEQELLELNKWASCWNWF |
| NO.: 33 | |
| SEQ ID | YTSLIHSLIEESQNQQEKNEQELLELNKWASLCNWF |
| NO.: 34 | |
| SEQ ID | YTSLIHSLIEESQNQQEKNEQELLELNKWASLWCWF |
| NO.: 35 | |
| SEQ ID | YTSLIHSLIEESQNQQEKNEQELLELNKWASLWNCF |
| NO.: 36 | |
| SEQ ID | YTSLIHSLIEESQNQQEKNEQELLELNKWASLWNWC |
| NO.: 37 | |
| SEQ ID | CYTSLIHSLIEESQNQQEKNEQELLELNKWASLWNWF |
| NO.: 38 | |
| SEQ ID | YTSLIHSLIEESQNQQEKNEQELLELNKWASLWNWC |
| NO.: 39 | |
| SEQ ID | Ac—CTSLIHSLIEESQNQQEKNEQELLELNKWASLWNW |
| NO.: 40 | F—NH₂ |
| SEQ ID | Ac—YCSLIHSLIEESQNQQEKNEQELLELNKWASLWNW |
| NO.: 41 | F—NH₂ |
| SEQ ID | Ac—YTCLIHSLIEESQNQQEKNEQELLELNKWASLWNW |
| NO.: 42 | F—NH₂ |
| SEQ ID | Ac—YTSCIHSLIEESQNQQEKNEQELLELNKWASLWNW |
| NO.: 43 | F—NH₂ |
| SEQ ID | Ac—YTSLCHSLIEESQNQQEKNEQELLELNKWASLWNW |
| NO.: 44 | F—NH₂ |
| SEQ ID | Ac—YTSLICSLIEESQNQQEKNEQELLELNKWASLWNW |
| NO.: 45 | F—NH₂ |
| SEQ ID | Ac—YTSLIHCLIEESQNQQEKNEQELLELNKWASLWNW |
| NO.: 46 | F—NH₂ |
| SEQ ID | Ac—YTSLIHSCIEESQNQQEKNEQELLELNKWASLWNW |
| NO.: 47 | F—NH₂ |
| SEQ ID | Ac—YTSLIHSCEESQNQQEKNEQELLELNKWASLWNW |
| NO.: 48 | F—NH₂ |
| SEQ ID | Ac—YTSLIHSLICESQNQQEKNEQELLELNKWASLWNW |
| NO.: 49 | F—NH₂ |
| SEQ ID | Ac—YTSLIHSLIECSQNQQEKNEQELLELNKWASLWNW |
| NO.: 50 | F—NH₂ |
| SEQ ID | Ac—YTSLIHSLIEECQNQQEKNEQELLELNKWASLWNW |
| NO.: 51 | F—NH₂ |
| SEQ ID | Ac—YTSLIHSLIEESCNQQEKNEQELLELNKWASLWNW |
| NO.: 52 | F—NH₂ |
| SEQ ID | Ac—YTSLIHSLIEESQCQQEKNEQELLELNKWASLWNW |
| NO.: 53 | F—NH₂ |
| SEQ ID | Ac—YTSLIHSLIEESQNCQEKNEQELLELNKWASLWNW |
| NO.: 54 | F—NH₂ |
| SEQ ID | Ac—YTSLIHSLIEESQNQCEKNEQELLELNKWASLWNW |
| NO.: 55 | F—NH₂ |
| SEQ ID | Ac—YTSLIHSLIEESQNQQCKNEQELLELNKWASLWNW |
| NO.: 56 | F—NH₂ |
| SEQ ID | Ac—YTSLIHSLIEESQNQQECNEQELLELNKWASLWNW |
| NO.: 57 | F—NH₂ |
| SEQ ID | Ac—YTSLIHSLIEESQNQQEKCEQELLELNKWASLWNW |
| NO.: 58 | F—NH₂ |
| SEQ ID | Ac—YTSLIHSLIEESQNQQEKNCQELLELNKWASLWNW |
| NO.: 59 | F—NH₂ |
| SEQ ID | Ac—YTSLIHSLIEESQNQQEKNECELLELNKWASLWNW |
| NO.: 60 | F—NH₂ |
| SEQ ID | Ac—YTSLIHSLIEESQNQQEKNEQCLLELNKWASLWNW |
| NO.: 61 | F—NH₂ |
| SEQ ID | Ac—YTSLIHSLIEESQNQQEKNEQECLELNKWASLWNW |
| NO.: 62 | F—NH₂ |
| SEQ ID | Ac—YTSLIHSLIEESQNQQEKNEQELCELNKWASLWNW |
| NO.: 63 | F—NH₂ |
| SEQ ID | Ac—YTSLIHSLIEESQNQQEKNEQELLCLNKWASLWNW |
| NO.: 64 | F—NH₂ |
| SEQ ID | Ac—YTSLIHSLIEESQNQQEKNEQELLECNKWASLWNW |
| NO.: 65 | F—NH₂ |
| SEQ ID | Ac—YTSLIHSLIEESQNQQEKNEQELLELCKWASLWNW |
| NO.: 66 | F—NH₂ |
| SEQ ID | Ac—YTSLIHSLIEESQNQQEKNEQELLELNCWASLWNW |
| NO.: 67 | F—NH₂ |
| SEQ ID | Ac—YTSLIHSLIEESQNQQEKNEQELLELNKCASLWNW |
| NO.: 68 | F—NH₂ |
| SEQ ID | Ac—YTSLIHSLIEESQNQQEKNEQELLELNKWCSLWNW |
| NO.: 69 | F—NH₂ |
| SEQ ID | Ac—YTSLIHSLIEESQNQQEKNEQELLELNKWACLWNW |
| NO.: 70 | F—NH₂ |
| SEQ ID | Ac—YTSLIHSLIEESQNQQEKNEQELLELNKWASCWNW |
| NO.: 71 | F—NH₂ |
| SEQ ID | Ac—YTSLIHSLIEESQNQQEKNEQELLELNKWASLCNW |
| NO.: 72 | F—NH₂ |
| SEQ ID | Ac—YTSLIHSLIEESQNQQEKNEQELLELNKWASLWCW |
| NO.: 73 | F—NH₂ |
| SEQ ID | Ac—YTSLIHSLIEESQNQQEKNEQELLELNKWASLWNC |
| NO.: 74 | F—NH₂ |
| SEQ ID | Ac—YTSLIHSLIEESQNQQEKNEQELLELNKWASLWNW |
| NO.: 75 | C—NH₂ |
| SEQ ID | Ac—CYTSLIHSLIEESQNQQEKNEQELLELNKWASLWNW |
| NO.: 76 | F—NH₂ |
| SEQ ID | Ac—YTSLIHSLIEESQNQQEKNEQELLELNKWASLWNW |
| NO.: 77 | F—NH₂ |

TABLE 2

| SEQ ID | |
|---|---|
| NO. | Sequence |
| SEQ ID | HGEGTFTSDLSKQMEEEAVRLFIEWLKNGGPSSGAPPPS |
| NO.: 78 | |
| SEQ ID | CGEGTFTSDLSKQMEEEAVRLFIEWLKNGGPSSGAPPPS |
| NO.: 79 | |
| SEQ ID | HCEGTFTSDLSKQMEEEAVRLFIEWLKNGGPSSGAPPPS |
| NO.: 80 | |
| SEQ ID | HGCGTFTSDLSKQMEEEAVRLFIEWLKNGGPSSGAPPPS |
| NO.: 81 | |
| SEQ ID | HGECTFTSDLSKQMEEEAVRLFIEWLKNGGPSSGAPPPS |
| NO.: 82 | |
| SEQ ID | HGEGCFTSDLSKQMEEEAVRLFIEWLKNGGPSSGAPPPS |
| NO.: 83 | |
| SEQ ID | HGEGTCTSDLSKQMEEEAVRLFIEWLKNGGPSSGAPPPS |
| NO.: 84 | |
| SEQ ID | HGEGTFCSDLSKQMEEEAVRLFIEWLKNGGPSSGAPPPS |
| NO.: 85 | |
| SEQ ID | HGEGTFTCDLSKQMEEEAVRLFIEWLKNGGPSSGAPPPS |
| NO.: 86 | |
| SEQ ID | HGEGTFTSCLSKQMEEEAVRLFIEWLKNGGPSSGAPPPS |
| NO.: 87 | |
| SEQ ID | HGEGTFTSDCSKQMEEEAVRLFIEWLKNGGPSSGAPPPS |
| NO.: 88 | |
| SEQ ID | HGEGTFTSDLCKQMEEEAVRLFIEWLKNGGPSSGAPPPS |
| NO.: 89 | |
| SEQ ID | HGEGTFTSDLSCQMEEEAVRLFIEWLKNGGPSSGAPPPS |
| NO.: 90 | |
| SEQ ID | HGEGTFTSDLSKCMEEEAVRLFIEWLKNGGPSSGAPPPS |
| NO.: 91 | |
| SEQ ID | HGEGTFTSDLSKQCEEEAVRLFIEWLKNGGPSSGAPPPS |
| NO.: 92 | |
| SEQ ID | HGEGTFTSDLSKQMCEEAVRLFIEWLKNGGPSSGAPPPS |
| NO.: 93 | |
| SEQ ID | HGEGTFTSDLSKQMECEAVRLFIEWLKNGGPSSGAPPPS |
| NO.: 94 | |
| SEQ ID | HGEGTFTSDLSKQMEECAVRLFIEWLKNGGPSSGAPPPS |
| NO.: 95 | |
| SEQ ID | HGEGTFTSDLSKQMEEECVRLFIEWLKNGGPSSGAPPPS |
| NO.: 96 | |
| SEQ ID | HGEGTFTSDLSKQMEEEACRLFIEWLKNGGPSSGAPPPS |
| NO.: 97 | |
| SEQ ID | HGEGTFTSDLSKQMEEEAVCLFIEWLKNGGPSSGAPPPS |
| NO.: 98 | |
| SEQ ID | HGEGTFTSDLSKQMEEEAVRCFIEWLKNGGPSSGAPPPS |
| NO.: 99 | |
| SEQ ID | HGEGTFTSDLSKQMEEEAVRLCIEWLKNGGPSSGAPPPS |
| NO.: 100 | |
| SEQ ID | HGEGTFTSDLSKQMEEEAVRLFCEWLKNGGPSSGAPPPS |
| NO.: 101 | |
| SEQ ID | HGEGTFTSDLSKQMEEEAVRLFICWLKNGGPSSGAPPPS |
| NO.: 102 | |
| SEQ ID | HGEGTFTSDLSKQMEEEAVRLFIECLKNGGPSSGAPPPS |
| NO.: 103 | |
| SEQ ID | HGEGTFTSDLSKQMEEEAVRLFIEWCKNGGPSSGAPPPS |
| NO.: 104 | |
| SEQ ID | HGEGTFTSDLSKQMEEEAVRLFIEWLCNGGPSSGAPPPS |
| NO.: 105 | |
| SEQ ID | HGEGTFTSDLSKQMEEEAVRLFIEWLKCGGPSSGAPPPS |
| NO.: 106 | |
| SEQ ID | HGEGTFTSDLSKQMEEEAVRLFIEWLKNCGPSSGAPPPS |
| NO.: 107 | |
| SEQ ID | HGEGTFTSDLSKQMEEEAVRLFIEWLKNGCPSSGAPPPS |
| NO.: 108 | |
| SEQ ID | HGEGTFTSDLSKQMEEEAVRLFIEWLKNGGCSSGAPPPS |
| NO.: 109 | |
| SEQ ID | HGEGTFTSDLSKQMEEEAVRLFIEWLKNGGPCSGAPPPS |
| NO.: 110 | |
| SEQ ID | HGEGTFTSDLSKQMEEEAVRLFIEWLKNGGPSCGAPPPS |
| NO.: 111 | |
| SEQ ID | HGEGTFTSDLSKQMEEEAVRLFIEWLKNGGPSSCAPPPS |
| NO.: 112 | |
| SEQ ID | HGEGTFTSDLSKQMEEEAVRLFIEWLKNGGPSSGCPPPS |
| NO.: 113 | |
| SEQ ID | HGEGTFTSDLSKQMEEEAVRLFIEWLKNGGPSSGACPPS |
| NO.: 114 | |
| SEQ ID | HGEGTFTSDLSKQMEEEAVRLFIEWLKNGGPSSGAPCPS |
| NO.: 115 | |
| SEQ ID | HGEGTFTSDLSKQMEEEAVRLFIEWLKNGGPSSGAPPCS |
| NO.: 116 | |
| SEQ ID | HGEGTFTSDLSKQMEEEAVRLFIEWLKNGGPSSGAPPPC |
| NO.: 117 | |
| SEQ ID | CHGEGTFTSDLSKQMEEEAVRLFIEWLKNGGPSSGAPPPS |
| NO.: 118 | |
| SEQ ID | HGEGTFTSDLSKQMEEEAVRLFIEWLKNGGPSSGAPPPSC |
| NO.: 119 | |
| SEQ ID | CGEGTFTSDLSKQMEEEAVRLFIEWLKNGGPSSGAPPP |
| NO.: 120 | S—NH₂ |
| SEQ ID | HCEGTFTSDLSKQMEEEAVRLFIEWLKNGGPSSGAPPP |
| NO.: 121 | S—NH₂ |
| SEQ ID | HGCGTFTSDLSKQMEEEAVRLFIEWLKNGGPSSGAPPP |
| NO.: 122 | S—NH₂ |
| SEQ ID | HGECTFTSDLSKQMEEEAVRLFIEWLKNGGPSSGAPPP |
| NO.: 123 | S—NH₂ |
| SEQ ID | HGEGCFTSDLSKQMEEEAVRLFIEWLKNGGPSSGAPPP |
| NO.: 124 | S—NH₂ |
| SEQ ID | HGEGTCTSDLSKQMEEEAVRLFIEWLKNGGPSSGAPPP |
| NO.: 125 | S—NH₂ |
| SEQ ID | HGEGTFCSDLSKQMEEEAVRLFIEWLKNGGPSSGAPPP |
| NO.: 126 | S—NH₂ |
| SEQ ID | HGEGTFTCDLSKQMEEEAVRLFIEWLKNGGPSSGAPPP |
| NO.: 127 | S—NH₂ |
| SEQ ID | HGEGTFTSCLSKQMEEEAVRLFIEWLKNGGPSSGAPPP |
| NO.: 128 | S—NH₂ |
| SEQ ID | HGEGTFTSDCSKQMEEEAVRLFIEWLKNGGPSSGAPPP |
| NO.: 129 | S—NH₂ |
| SEQ ID | HGEGTFTSDLCKQMEEEAVRLFIEWLKNGGPSSGAPPP |
| NO.: 130 | S—NH₂ |
| SEQ ID | HGEGTFTSDLSCQMEEEAVRLFIEWLKNGGPSSGAPPP |
| NO.: 131 | S—NH₂ |
| SEQ ID | HGEGTFTSDLSKCMEEEAVRLFIEWLKNGGPSSGAPPP |
| NO.: 132 | S—NH₂ |
| SEQ ID | HGEGTFTSDLSKQCEEEAVRLFIEWLKNGGPSSGAPPP |
| NO.: 133 | S—NH₂ |
| SEQ ID | HGEGTFTSDLSKQMCEEAVRLFIEWLKNGGPSSGAPPP |
| NO.: 134 | S—NH₂ |
| SEQ ID | HGEGTFTSDLSKQMECEAVRLFIEWLKNGGPSSGAPPP |
| NO.: 135 | S—NH₂ |
| SEQ ID | HGEGTFTSDLSKQMEECAVRLFIEWLKNGGPSSGAPPP |
| NO.: 136 | S—NH₂ |
| SEQ ID | HGEGTFTSDLSKQMEEECVRLFIEWLKNGGPSSGAPPP |
| NO.: 137 | S—NH₂ |
| SEQ ID | HGEGTFTSDLSKQMEEEACRLFIEWLKNGGPSSGAPPP |
| NO.: 138 | S—NH₂ |
| SEQ ID | HGEGTFTSDLSKQMEEEAVCLFIEWLKNGGPSSGAPPP |
| NO.: 139 | S—NH₂ |
| SEQ ID | HGEGTFTSDLSKQMEEEAVRCFIEWLKNGGPSSGAPPP |
| NO.: 140 | S—NH₂ |
| SEQ ID | HGEGTFTSDLSKQMEEEAVRLCIEWLKNGGPSSGAPPP |
| NO.: 141 | S—NH₂ |
| SEQ ID | HGEGTFTSDLSKQMEEEAVRLFCEWLKNGGPSSGAPPP |
| NO.: 142 | S—NH₂ |
| SEQ ID | HGEGTFTSDLSKQMEEEAVRLFICWLKNGGPSSGAPPP |
| NO.: 143 | S—NH₂ |
| SEQ ID | HGEGTFTSDLSKQMEEEAVRLFIECLKNGGPSSGAPPP |
| NO.: 144 | S—NH₂ |
| SEQ ID | HGEGTFTSDLSKQMEEEAVRLFIEWCKNGGPSSGAPPP |
| NO.: 145 | S—NH₂ |
| SEQ ID | HGEGTFTSDLSKQMEEEAVRLFIEWLCNGGPSSGAPPP |
| NO.: 146 | S—NH₂ |
| SEQ ID | HGEGTFTSDLSKQMEEEAVRLFIEWLKCGGPSSGAPPP |
| NO.: 147 | S—NH₂ |
| SEQ ID | HGEGTFTSDLSKQMEEEAVRLFIEWLKNCGPSSGAPPP |
| NO.: 148 | S—NH₂ |
| SEQ ID | HGEGTFTSDLSKQMEEEAVRLFIEWLKNGCPSSGAPPP |
| NO.: 149 | S—NH₂ |
| SEQ ID | HGEGTFTSDLSKQMEEEAVRLFIEWLKNGGCSSGAPPP |
| NO.: 150 | S—NH₂ |
| SEQ ID | HGEGTFTSDLSKQMEEEAVRLFIEWLKNGGPCSGAPPP |
| NO.: 151 | S—NH₂ |
| SEQ ID | HGEGTFTSDLSKQMEEEAVRLFIEWLKNGGPSCGAPPP |
| NO.: 152 | S—NH₂ |
| SEQ ID | HGEGTFTSDLSKQMEEEAVRLFIEWLKNGGPSSCAPPP |
| NO.: 153 | S—NH₂ |
| SEQ ID | HGEGTFTSDLSKQMEEEAVRLFIEWLKNGGPSSGCPPP |
| NO.: 154 | S—NH₂ |
| SEQ ID | HGEGTFTSDLSKQMEEEAVRLFIEWLKNGGPSSGACPP |
| NO.: 155 | S—NH₂ |
| SEQ ID | HGEGTFTSDLSKQMEEEAVRLFIEWLKNGGPSSGAPCP |
| NO.: 156 | S—NH₂ |
| SEQ ID | HGEGTFTSDLSKQMEEEAVRLFIEWLKNGGPSSGAPPC |
| NO.: 157 | S—NH₂ |
| SEQ ID | HGEGTFTSDLSKQMEEEAVRLFIEWLKNGGPSSGAPPP |
| NO.: 158 | C—NH₂ |
| SEQ ID | CHGEGTFTSDLSKQMEEEAVRLFIEWLKNGGPSSGAPPP |
| NO.: 159 | S—NH₂ |
| SEQ ID | HGEGTFTSDLSKQMEEEAVRLFIEWLKNGGPSSGAPPPS |
| NO.: 160 | C—NH₂ |

1-51. (canceled)

52. A pharmaceutical composition comprising:
(i) a compound of formula 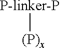
or a pharmaceutically acceptable salt thereof; and
(ii) one or more pharmaceutically acceptable excipients;
wherein:
P is a therapeutic polypeptide, or an analog thereof, covalently linked to a linker; and
x is 0 or 1.

53. The pharmaceutical composition of claim 52, wherein x is 0.

54. The pharmaceutical composition of claim 52, wherein the linker is selected from a group consisting of polyethylene glycol, polypropylene glycol, polyamine, polyamide, polyurethane, polyester, and combinations thereof.

55. The pharmaceutical composition of claim 52, wherein each P comprises at least one Cys residue.

56. The pharmaceutical composition of claim 52, wherein the linker is L-(CH₂CH₂O)ₙ—CH₂CH₂-L, wherein n is an integer and each L is independently a reactive group.

57. The pharmaceutical composition of 56, wherein each L is independently selected from a group consisting of 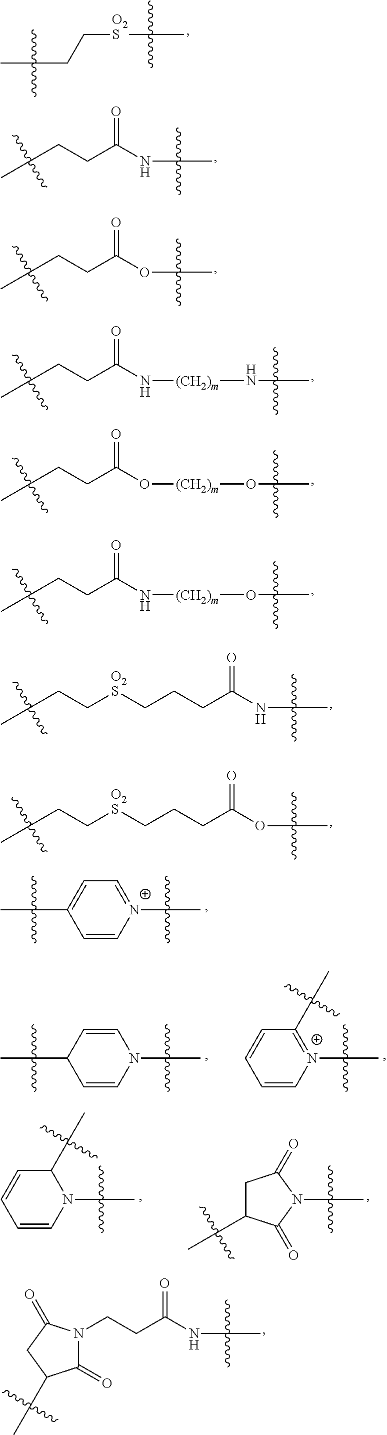 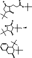
wherein each m is independently an integer from 2 to 10.

58. The pharmaceutical composition of claim 52, wherein the compound of formula is:

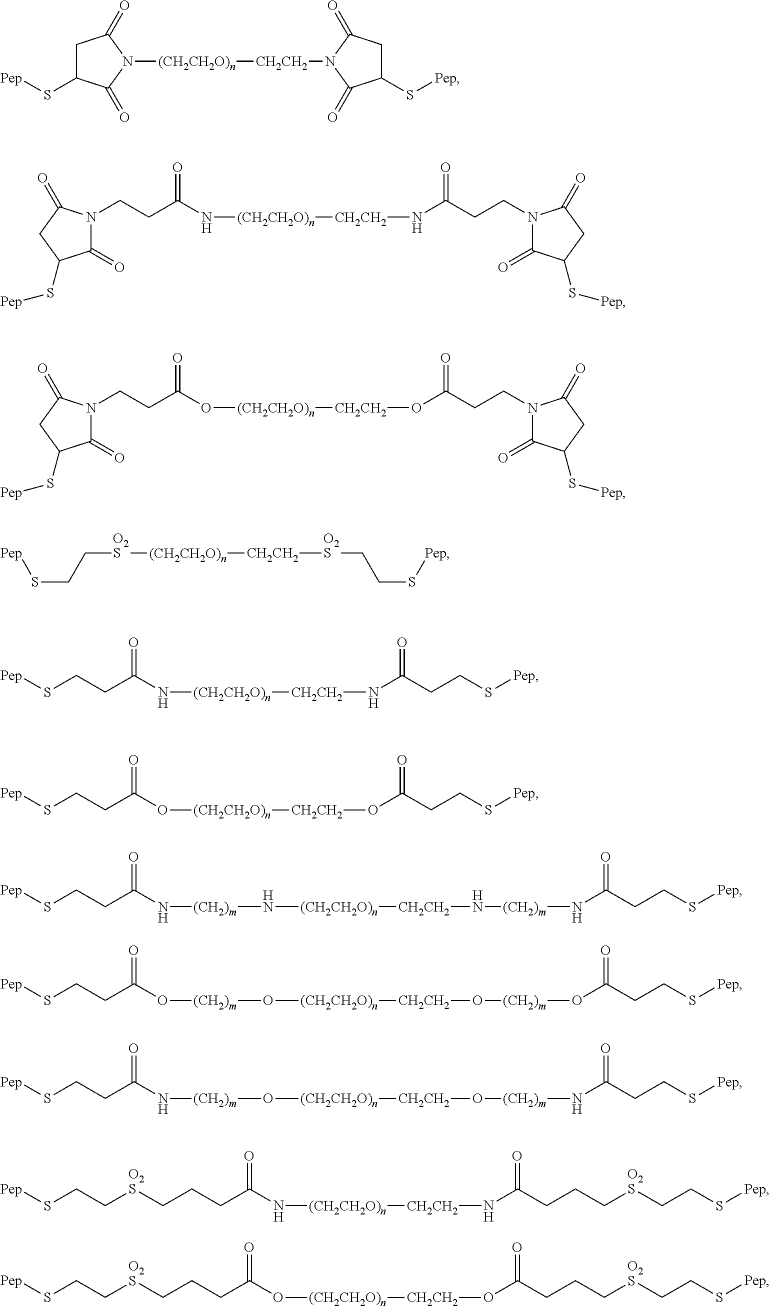

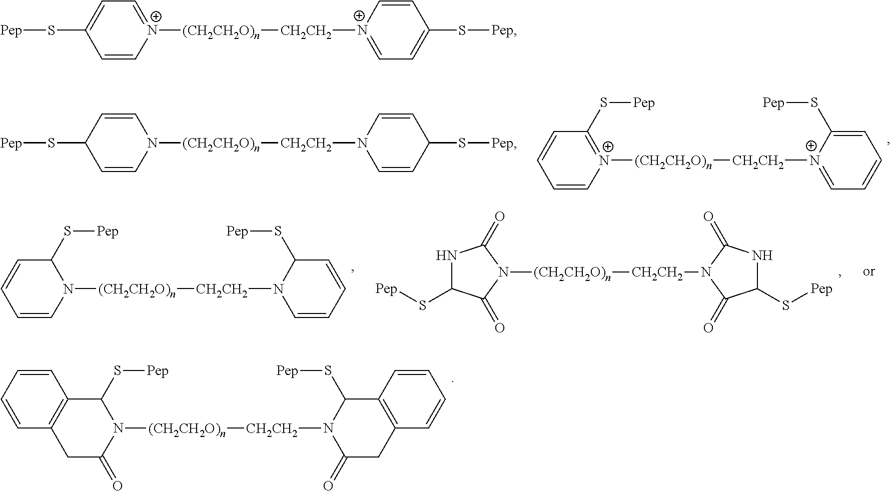

59. The pharmaceutical composition of claim 52, wherein each P is independently enfuvirtide or an analog thereof.

60. The pharmaceutical composition of claim 59, wherein the enfuvirtide analog has greater than about 90% sequence homology with enfuvirtide.

61. The pharmaceutical composition of claim 52, wherein each P is independently insulin or an analog thereof.

62. The pharmaceutical composition of claim 61, wherein the insulin analog has greater than about 90% sequence homology with insulin.

63. The pharmaceutical composition of claim 52, wherein each P is independently selected from a group consisting of exenatide, glucagon-like peptide-1 (GLP-1), insulin, lisxisenatide and analogs thereof.

64. The pharmaceutical composition of claim 63, wherein the analogs of exenatide, glucagon-like peptide-1 (GLP-1), insulin and lisxisenatide have greater than about 90% sequence homology with exenatide, glucagon-like peptide-1 (GLP-1), insulin and lisxisenatide respectively.

65. The pharmaceutical composition of claim 52, wherein the compound is:

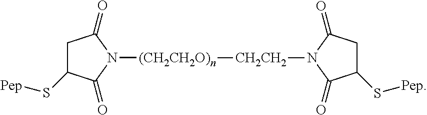

66. The pharmaceutical composition of claim 52 or 53, wherein the linker has a molecular weight selected from a group consisting of about 5,000 Daltons, about 6,000 Daltons, about 7,000 Daltons, about 8,000 Daltons, about 9,000 Daltons, about 10,000 Daltons, about 12,000 Daltons, about 14,000 Daltons, about 16,000 Daltons, about 18,000 Daltons, and about 20,000 Daltons.

67. The pharmaceutical composition of claim 52 or 53, wherein the linker comprises two carbon monomer and wherein number of the two carbon monomers in the linker is 1, 2, 3, 4, 5, 6, 7, 8, 9, 10, 11, 12, 13, 14, 15, 16, 17, 18, 19, 20, 21, 22, 23, 24, 25, or 26.

68. The pharmaceutical composition of claim 52, wherein the compound has an in vivo elimination half-life selected from a group consisting of greater than about 6 hours, greater than about 12 hours, and greater than about 18 hours.

69. A method of treating a disease or disorder, the method comprising administering to a subject a therapeutically effective amount of a pharmaceutical composition comprising:
(i) a compound of formula 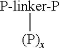
or a pharmaceutically acceptable salt thereof; and
(ii) one or more pharmaceutically-acceptable excipients;
wherein:
each P is a therapeutic peptide, or an analog thereof; and
x is 0 or 1.

70. The method of claim 69, wherein x is 0.

71. The method of claim 69, wherein the linker is selected from a group consisting of polyethylene glycol, polypropylene glycol, polyamine, polyamide, polyurethane, polyester, and combinations thereof.

72. The method of claim 69, wherein each P comprises at least one Cys residue.

73. The method of claim 69, wherein the linker is L-(CH₂CH₂O)ₙ—CH₂CH₂-L, wherein n is an integer and each L is independently a reactive group.

74. The method of claim 73, wherein each L is independently selected from a group consisting of 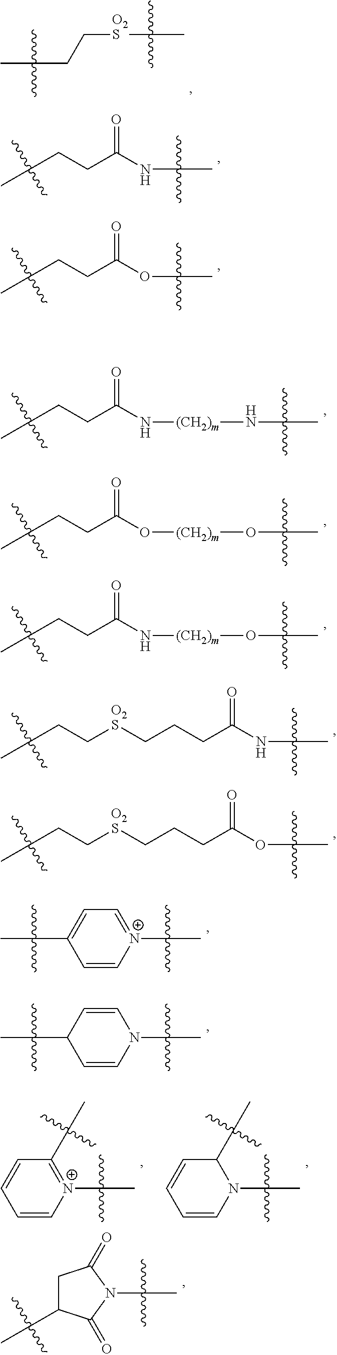 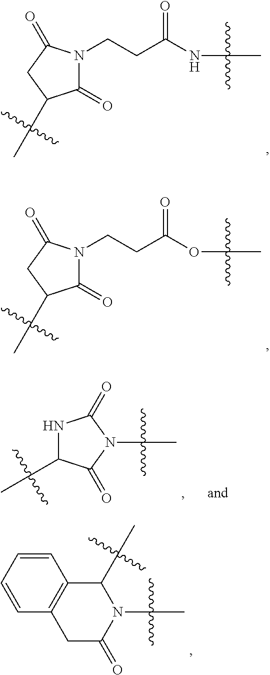
wherein each m is independently an integer from 2 to 10.

75. The method of claim 69, wherein the compound:

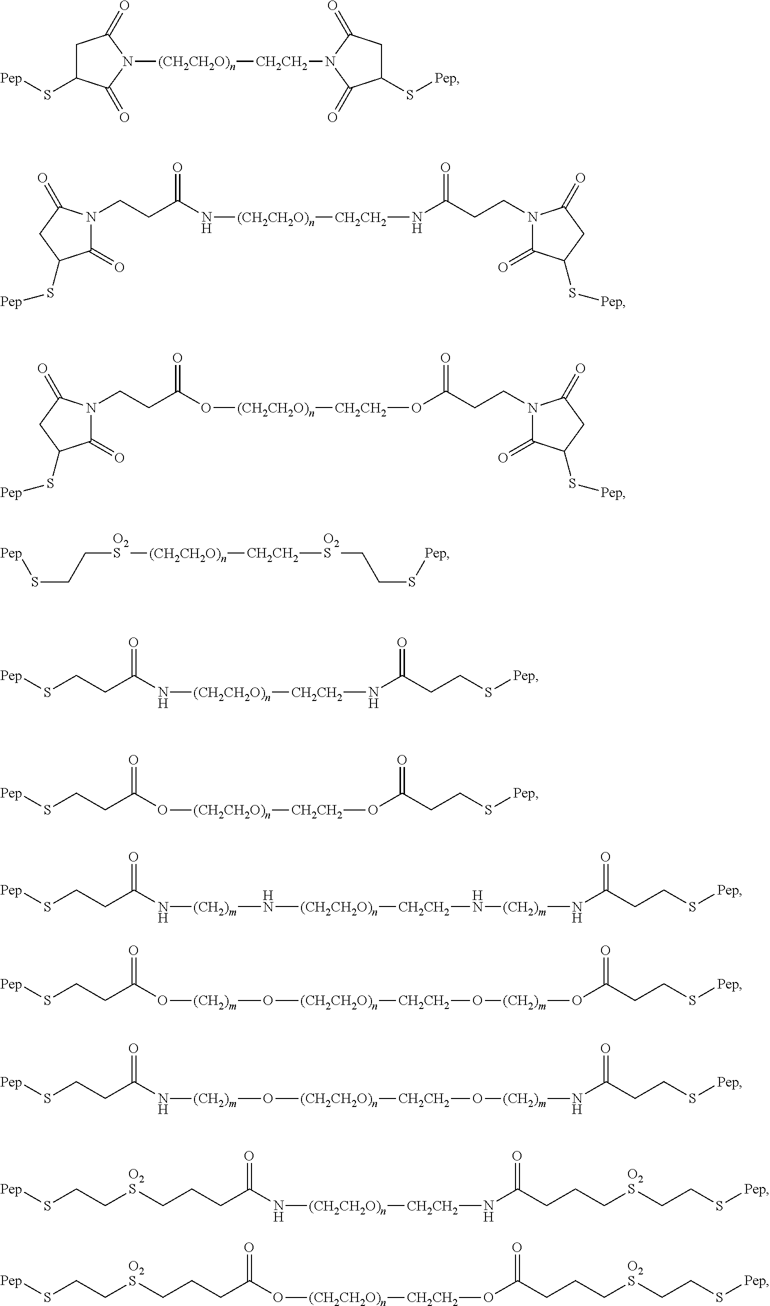

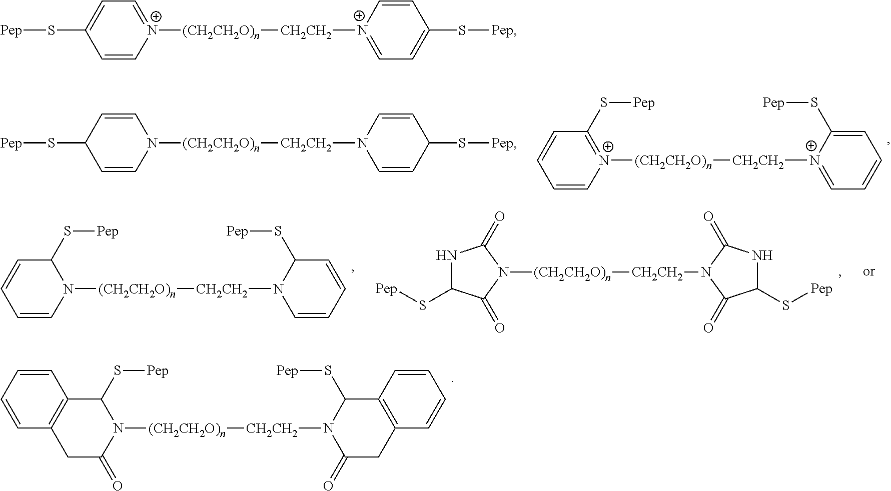

76. The method of claim 69, wherein the disease or disorder is HIV.

77. The method of claim 69, wherein the disease or disorder is AIDS.

78. The method of claim 69, wherein the disease or disorder is diabetes.

79. The method of claim 69, wherein each P is independently a peptide selected from a group consisting of exenatide, glucagon-like peptide-1 (GLP-1), insulin, lixisenatide, and analogs thereof.

80. The method of claim 79, wherein the analogs of exenatide, glucagon-like peptide-1 (GLP-1), insulin, and lixisenatide have greater than about 90% sequence homology with exenatide, glucagon-like peptide-1 (GLP-1), insulin, and lixisenatide respectively.

81. The method of claim 69, wherein at least one P is insulin or an analog thereof.

82. The method of claim 81, wherein the insulin analog has greater than about 90% sequence homology with insulin.

83. The method of claim 69, wherein at least one P is enfuvirtide or an analog thereof.

84. The method of claim 83, wherein the enfuvirtide analog has greater than about 90% sequence homology with enfuvirtide.

85. The method of claim 69 or 70, wherein the linker has a molecular weight selected from a group consisting of about 5,000 Daltons, about 6,000 Daltons, about 7,000 Daltons, about 8,000 Daltons, about 9,000 Daltons, about 10,000 Daltons, about 12,000 Daltons, about 14,000 Daltons, about 16,000 Daltons, about 18,000 Daltons, and about 20,000 Daltons.

86. The method of claim 69 or 70, wherein the linker comprises two carbon monomer and wherein number of the two carbon monomers in the linker is 1, 2, 3, 4, 5, 6, 7, 8, 9, 10, 11, 12, 13, 14, 15, 16, 17, 18, 19, 20, 21, 22, 23, 24, 25, or 26.


SEQUENCE LISTING (167 sequences)

SEQ ID NO: 1 - protein, 36 aa, Artificial Sequence
  Description of Artificial Sequence Synthetic polypeptide
YTSLIHSLIEESQNQQEKNEQELLELNKWASLWNWF

SEQ ID NO: 2 - protein, 36 aa, Artificial Sequence
  Description of Artificial Sequence Synthetic polypeptide
CTSLIHSLIEESQNQQEKNEQELLELNKWASLWNWF

SEQ ID NO: 3 - protein, 36 aa, Artificial Sequence
  Description of Artificial Sequence Synthetic polypeptide
YCSLIHSLIEESQNQQEKNEQELLELNKWASLWNWF

SEQ ID NO: 4 - protein, 36 aa, Artificial Sequence
  Description of Artificial Sequence Synthetic polypeptide
YTCLIHSLIEESQNQQEKNEQELLELNKWASLWNWF

SEQ ID NO: 5 - protein, 36 aa, Artificial Sequence
  Description of Artificial Sequence Synthetic polypeptide
YTSCIHSLIEESQNQQEKNEQELLELNKWASLWNWF

SEQ ID NO: 6 - protein, 36 aa, Artificial Sequence
  Description of Artificial Sequence Synthetic polypeptide
YTSLCHSLIEESQNQQEKNEQELLELNKWASLWNWF

SEQ ID NO: 7 - protein, 36 aa, Artificial Sequence
  Description of Artificial Sequence Synthetic polypeptide
YTSLICSLIEESQNQQEKNEQELLELNKWASLWNWF

SEQ ID NO: 8 - protein, 36 aa, Artificial Sequence
  Description of Artificial Sequence Synthetic polypeptide
YTSLIHCLIEESQNQQEKNEQELLELNKWASLWNWF

SEQ ID NO: 9 - protein, 36 aa, Artificial Sequence
  Description of Artificial Sequence Synthetic polypeptide
YTSLIHSCIEESQNQQEKNEQELLELNKWASLWNWF

SEQ ID NO: 10 - protein, 35 aa, Artificial Sequence
  Description of Artificial Sequence Synthetic polypeptide
YTSLIHSCEESQNQQEKNEQELLELNKWASLWNWF

SEQ ID NO: 11 - protein, 36 aa, Artificial Sequence
  Description of Artificial Sequence Synthetic polypeptide
YTSLIHSLICESQNQQEKNEQELLELNKWASLWNWF

SEQ ID NO: 12 - protein, 36 aa, Artificial Sequence
  Description of Artificial Sequence Synthetic polypeptide
YTSLIHSLIECSQNQQEKNEQELLELNKWASLWNWF

SEQ ID NO: 13 - protein, 36 aa, Artificial Sequence
  Description of Artificial Sequence Synthetic polypeptide
YTSLIHSLIEECQNQQEKNEQELLELNKWASLWNWF

SEQ ID NO: 14 - protein, 36 aa, Artificial Sequence
  Description of Artificial Sequence Synthetic polypeptide
YTSLIHSLIEESCNQQEKNEQELLELNKWASLWNWF

SEQ ID NO: 15 - protein, 36 aa, Artificial Sequence
  Description of Artificial Sequence Synthetic polypeptide
YTSLIHSLIEESQCQQEKNEQELLELNKWASLWNWF

SEQ ID NO: 16 - protein, 36 aa, Artificial Sequence
  Description of Artificial Sequence Synthetic polypeptide
YTSLIHSLIEESQNCQEKNEQELLELNKWASLWNWF

SEQ ID NO: 17 - protein, 36 aa, Artificial Sequence
  Description of Artificial Sequence Synthetic polypeptide
YTSLIHSLIEESQNQCEKNEQELLELNKWASLWNWF

SEQ ID NO: 18 - protein, 36 aa, Artificial Sequence
  Description of Artificial Sequence Synthetic polypeptide
YTSLIHSLIEESQNQQCKNEQELLELNKWASLWNWF

SEQ ID NO: 19 - protein, 36 aa, Artificial Sequence
  Description of Artificial Sequence Synthetic polypeptide
YTSLIHSLIEESQNQQECNEQELLELNKWASLWNWF

SEQ ID NO: 20 - protein, 36 aa, Artificial Sequence
  Description of Artificial Sequence Synthetic polypeptide
YTSLIHSLIEESQNQQEKCEQELLELNKWASLWNWF

SEQ ID NO: 21 - protein, 36 aa, Artificial Sequence
  Description of Artificial Sequence Synthetic polypeptide
YTSLIHSLIEESQNQQEKNCQELLELNKWASLWNWF

SEQ ID NO: 22 - protein, 36 aa, Artificial Sequence
  Description of Artificial Sequence Synthetic polypeptide
YTSLIHSLIEESQNQQEKNECELLELNKWASLWNWF

SEQ ID NO: 23 - protein, 36 aa, Artificial Sequence
  Description of Artificial Sequence Synthetic polypeptide
YTSLIHSLIEESQNQQEKNEQCLLELNKWASLWNWF

SEQ ID NO: 24 - protein, 36 aa, Artificial Sequence
  Description of Artificial Sequence Synthetic polypeptide
YTSLIHSLIEESQNQQEKNEQECLELNKWASLWNWF

SEQ ID NO: 25 - protein, 36 aa, Artificial Sequence
  Description of Artificial Sequence Synthetic polypeptide
YTSLIHSLIEESQNQQEKNEQELCELNKWASLWNWF

SEQ ID NO: 26 - protein, 36 aa, Artificial Sequence
  Description of Artificial Sequence Synthetic polypeptide
YTSLIHSLIEESQNQQEKNEQELLCLNKWASLWNWF

SEQ ID NO: 27 - protein, 36 aa, Artificial Sequence
  Description of Artificial Sequence Synthetic polypeptide
YTSLIHSLIEESQNQQEKNEQELLECNKWASLWNWF

SEQ ID NO: 28 - protein, 36 aa, Artificial Sequence
  Description of Artificial Sequence Synthetic polypeptide
YTSLIHSLIEESQNQQEKNEQELLELCKWASLWNWF

SEQ ID NO: 29 - protein, 36 aa, Artificial Sequence
  Description of Artificial Sequence Synthetic polypeptide
YTSLIHSLIEESQNQQEKNEQELLELNCWASLWNWF

SEQ ID NO: 30 - protein, 36 aa, Artificial Sequence
  Description of Artificial Sequence Synthetic polypeptide
YTSLIHSLIEESQNQQEKNEQELLELNKCASLWNWF

SEQ ID NO: 31 - protein, 36 aa, Artificial Sequence
  Description of Artificial Sequence Synthetic polypeptide
YTSLIHSLIEESQNQQEKNEQELLELNKWCSLWNWF

SEQ ID NO: 32 - protein, 36 aa, Artificial Sequence
  Description of Artificial Sequence Synthetic polypeptide
YTSLIHSLIEESQNQQEKNEQELLELNKWACLWNWF

SEQ ID NO: 33 - protein, 36 aa, Artificial Sequence
  Description of Artificial Sequence Synthetic polypeptide
YTSLIHSLIEESQNQQEKNEQELLELNKWASCWNWF

SEQ ID NO: 34 - protein, 36 aa, Artificial Sequence
  Description of Artificial Sequence Synthetic polypeptide
YTSLIHSLIEESQNQQEKNEQELLELNKWASLCNWF

SEQ ID NO: 35 - protein, 36 aa, Artificial Sequence
  Description of Artificial Sequence Synthetic polypeptide
YTSLIHSLIEESQNQQEKNEQELLELNKWASLWCWF

SEQ ID NO: 36 - protein, 36 aa, Artificial Sequence
  Description of Artificial Sequence Synthetic polypeptide
YTSLIHSLIEESQNQQEKNEQELLELNKWASLWNCF

SEQ ID NO: 37 - protein, 36 aa, Artificial Sequence
  Description of Artificial Sequence Synthetic polypeptide
YTSLIHSLIEESQNQQEKNEQELLELNKWASLWNWC

SEQ ID NO: 38 - protein, 37 aa, Artificial Sequence
  Description of Artificial Sequence Synthetic polypeptide
CYTSLIHSLIEESQNQQEKNEQELLELNKWASLWNWF

SEQ ID NO: 39 - protein, 37 aa, Artificial Sequence
  Description of Artificial Sequence Synthetic polypeptide
YTSLIHSLIEESQNQQEKNEQELLELNKWASLWNWFC

SEQ ID NO: 40 - protein, 36 aa, Artificial Sequence; identical to SEQ ID NO: 2
  Description of Artificial Sequence Synthetic polypeptide

SEQ ID NO: 41 - protein, 36 aa, Artificial Sequence; identical to SEQ ID NO: 3
  Description of Artificial Sequence Synthetic polypeptide

SEQ ID NO: 42 - protein, 36 aa, Artificial Sequence; identical to SEQ ID NO: 4
  Description of Artificial Sequence Synthetic polypeptide

SEQ ID NO: 43 - protein, 36 aa, Artificial Sequence; identical to SEQ ID NO: 5
  Description of Artificial Sequence Synthetic polypeptide

SEQ ID NO: 44 - protein, 36 aa, Artificial Sequence; identical to SEQ ID NO: 6
  Description of Artificial Sequence Synthetic polypeptide

SEQ ID NO: 45 - protein, 36 aa, Artificial Sequence; identical to SEQ ID NO: 7
  Description of Artificial Sequence Synthetic polypeptide

SEQ ID NO: 46 - protein, 36 aa, Artificial Sequence; identical to SEQ ID NO: 8
  Description of Artificial Sequence Synthetic polypeptide

SEQ ID NO: 47 - protein, 36 aa, Artificial Sequence; identical to SEQ ID NO: 9
  Description of Artificial Sequence Synthetic polypeptide

SEQ ID NO: 48 - protein, 35 aa, Artificial Sequence; identical to SEQ ID NO: 10
  Description of Artificial Sequence Synthetic polypeptide

SEQ ID NO: 49 - protein, 36 aa, Artificial Sequence; identical to SEQ ID NO: 11
  Description of Artificial Sequence Synthetic polypeptide

SEQ ID NO: 50 - protein, 36 aa, Artificial Sequence; identical to SEQ ID NO: 12
  Description of Artificial Sequence Synthetic polypeptide

SEQ ID NO: 51 - protein, 36 aa, Artificial Sequence; identical to SEQ ID NO: 13
  Description of Artificial Sequence Synthetic polypeptide

SEQ ID NO: 52 - protein, 36 aa, Artificial Sequence; identical to SEQ ID NO: 14
  Description of Artificial Sequence Synthetic polypeptide

SEQ ID NO: 53 - protein, 36 aa, Artificial Sequence; identical to SEQ ID NO: 15
  Description of Artificial Sequence Synthetic polypeptide

SEQ ID NO: 54 - protein, 36 aa, Artificial Sequence; identical to SEQ ID NO: 16
  Description of Artificial Sequence Synthetic polypeptide

SEQ ID NO: 55 - protein, 36 aa, Artificial Sequence; identical to SEQ ID NO: 17
  Description of Artificial Sequence Synthetic polypeptide

SEQ ID NO: 56 - protein, 36 aa, Artificial Sequence; identical to SEQ ID NO: 18
  Description of Artificial Sequence Synthetic polypeptide

SEQ ID NO: 57 - protein, 36 aa, Artificial Sequence; identical to SEQ ID NO: 19
  Description of Artificial Sequence Synthetic polypeptide

SEQ ID NO: 58 - protein, 36 aa, Artificial Sequence; identical to SEQ ID NO: 20
  Description of Artificial Sequence Synthetic polypeptide

SEQ ID NO: 59 - protein, 36 aa, Artificial Sequence; identical to SEQ ID NO: 21
  Description of Artificial Sequence Synthetic polypeptide

SEQ ID NO: 60 - protein, 36 aa, Artificial Sequence; identical to SEQ ID NO: 22
  Description of Artificial Sequence Synthetic polypeptide

SEQ ID NO: 61 - protein, 36 aa, Artificial Sequence; identical to SEQ ID NO: 23
  Description of Artificial Sequence Synthetic polypeptide

SEQ ID NO: 62 - protein, 36 aa, Artificial Sequence; identical to SEQ ID NO: 24
  Description of Artificial Sequence Synthetic polypeptide

SEQ ID NO: 63 - protein, 36 aa, Artificial Sequence; identical to SEQ ID NO: 25
  Description of Artificial Sequence Synthetic polypeptide

SEQ ID NO: 64 - protein, 36 aa, Artificial Sequence; identical to SEQ ID NO: 26
  Description of Artificial Sequence Synthetic polypeptide

SEQ ID NO: 65 - protein, 36 aa, Artificial Sequence; identical to SEQ ID NO: 27
  Description of Artificial Sequence Synthetic polypeptide

SEQ ID NO: 66 - protein, 36 aa, Artificial Sequence; identical to SEQ ID NO: 28
  Description of Artificial Sequence Synthetic polypeptide

SEQ ID NO: 67 - protein, 36 aa, Artificial Sequence; identical to SEQ ID NO: 29
  Description of Artificial Sequence Synthetic polypeptide

SEQ ID NO: 68 - protein, 36 aa, Artificial Sequence; identical to SEQ ID NO: 30
  Description of Artificial Sequence Synthetic polypeptide

SEQ ID NO: 69 - protein, 36 aa, Artificial Sequence; identical to SEQ ID NO: 31
  Description of Artificial Sequence Synthetic polypeptide

SEQ ID NO: 70 - protein, 36 aa, Artificial Sequence; identical to SEQ ID NO: 32
  Description of Artificial Sequence Synthetic polypeptide

SEQ ID NO: 71 - protein, 36 aa, Artificial Sequence; identical to SEQ ID NO: 33
  Description of Artificial Sequence Synthetic polypeptide

SEQ ID NO: 72 - protein, 36 aa, Artificial Sequence; identical to SEQ ID NO: 34
  Description of Artificial Sequence Synthetic polypeptide

SEQ ID NO: 73 - protein, 36 aa, Artificial Sequence; identical to SEQ ID NO: 35
  Description of Artificial Sequence Synthetic polypeptide

SEQ ID NO: 74 - protein, 36 aa, Artificial Sequence; identical to SEQ ID NO: 36
  Description of Artificial Sequence Synthetic polypeptide

SEQ ID NO: 75 - protein, 36 aa, Artificial Sequence; identical to SEQ ID NO: 37
  Description of Artificial Sequence Synthetic polypeptide

SEQ ID NO: 76 - protein, 37 aa, Artificial Sequence; identical to SEQ ID NO: 38
  Description of Artificial Sequence Synthetic polypeptide

SEQ ID NO: 77 - protein, 37 aa, Artificial Sequence; identical to SEQ ID NO: 39
  Description of Artificial Sequence Synthetic polypeptide

SEQ ID NO: 78 - protein, 39 aa, Artificial Sequence
  Description of Artificial Sequence Synthetic polypeptide
HGEGTFTSDLSKQMEEEAVRLFIEWLKNGGPSSGAPPPS

SEQ ID NO: 79 - protein, 39 aa, Artificial Sequence
  Description of Artificial Sequence Synthetic polypeptide
CGEGTFTSDLSKQMEEEAVRLFIEWLKNGGPSSGAPPPS

SEQ ID NO: 80 - protein, 39 aa, Artificial Sequence
  Description of Artificial Sequence Synthetic polypeptide
HCEGTFTSDLSKQMEEEAVRLFIEWLKNGGPSSGAPPPS

SEQ ID NO: 81 - protein, 39 aa, Artificial Sequence
  Description of Artificial Sequence Synthetic polypeptide
HGCGTFTSDLSKQMEEEAVRLFIEWLKNGGPSSGAPPPS

SEQ ID NO: 82 - protein, 39 aa, Artificial Sequence
  Description of Artificial Sequence Synthetic polypeptide
HGECTFTSDLSKQMEEEAVRLFIEWLKNGGPSSGAPPPS

SEQ ID NO: 83 - protein, 39 aa, Artificial Sequence
  Description of Artificial Sequence Synthetic polypeptide
HGEGCFTSDLSKQMEEEAVRLFIEWLKNGGPSSGAPPPS

SEQ ID NO: 84 - protein, 39 aa, Artificial Sequence
  Description of Artificial Sequence Synthetic polypeptide
HGEGTCTSDLSKQMEEEAVRLFIEWLKNGGPSSGAPPPS

SEQ ID NO: 85 - protein, 39 aa, Artificial Sequence
  Description of Artificial Sequence Synthetic polypeptide
HGEGTFCSDLSKQMEEEAVRLFIEWLKNGGPSSGAPPPS

SEQ ID NO: 86 - protein, 39 aa, Artificial Sequence
  Description of Artificial Sequence Synthetic polypeptide
HGEGTFTCDLSKQMEEEAVRLFIEWLKNGGPSSGAPPPS

SEQ ID NO: 87 - protein, 39 aa, Artificial Sequence
  Description of Artificial Sequence Synthetic polypeptide
HGEGTFTSCLSKQMEEEAVRLFIEWLKNGGPSSGAPPPS

SEQ ID NO: 88 - protein, 39 aa, Artificial Sequence
  Description of Artificial Sequence Synthetic polypeptide
HGEGTFTSDCSKQMEEEAVRLFIEWLKNGGPSSGAPPPS

SEQ ID NO: 89 - protein, 39 aa, Artificial Sequence
  Description of Artificial Sequence Synthetic polypeptide
HGEGTFTSDLCKQMEEEAVRLFIEWLKNGGPSSGAPPPS

SEQ ID NO: 90 - protein, 39 aa, Artificial Sequence
  Description of Artificial Sequence Synthetic polypeptide
HGEGTFTSDLSCQMEEEAVRLFIEWLKNGGPSSGAPPPS

SEQ ID NO: 91 - protein, 39 aa, Artificial Sequence
  Description of Artificial Sequence Synthetic polypeptide
HGEGTFTSDLSKCMEEEAVRLFIEWLKNGGPSSGAPPPS

SEQ ID NO: 92 - protein, 39 aa, Artificial Sequence
  Description of Artificial Sequence Synthetic polypeptide
HGEGTFTSDLSKQCEEEAVRLFIEWLKNGGPSSGAPPPS

SEQ ID NO: 93 - protein, 39 aa, Artificial Sequence
  Description of Artificial Sequence Synthetic polypeptide
HGEGTFTSDLSKQMCEEAVRLFIEWLKNGGPSSGAPPPS

SEQ ID NO: 94 - protein, 39 aa, Artificial Sequence
  Description of Artificial Sequence Synthetic polypeptide
HGEGTFTSDLSKQMECEAVRLFIEWLKNGGPSSGAPPPS

SEQ ID NO: 95 - protein, 39 aa, Artificial Sequence
  Description of Artificial Sequence Synthetic polypeptide
HGEGTFTSDLSKQMEECAVRLFIEWLKNGGPSSGAPPPS

SEQ ID NO: 96 - protein, 39 aa, Artificial Sequence
  Description of Artificial Sequence Synthetic polypeptide
HGEGTFTSDLSKQMEEECVRLFIEWLKNGGPSSGAPPPS

SEQ ID NO: 97 - protein, 39 aa, Artificial Sequence
  Description of Artificial Sequence Synthetic polypeptide
HGEGTFTSDLSKQMEEEACRLFIEWLKNGGPSSGAPPPS

SEQ ID NO: 98 - protein, 39 aa, Artificial Sequence
  Description of Artificial Sequence Synthetic polypeptide
HGEGTFTSDLSKQMEEEAVCLFIEWLKNGGPSSGAPPPS

SEQ ID NO: 99 - protein, 39 aa, Artificial Sequence
  Description of Artificial Sequence Synthetic polypeptide
HGEGTFTSDLSKQMEEEAVRCFIEWLKNGGPSSGAPPPS

SEQ ID NO: 100 - protein, 39 aa, Artificial Sequence
  Description of Artificial Sequence Synthetic polypeptide
HGEGTFTSDLSKQMEEEAVRLCIEWLKNGGPSSGAPPPS

SEQ ID NO: 101 - protein, 39 aa, Artificial Sequence
  Description of Artificial Sequence Synthetic polypeptide
HGEGTFTSDLSKQMEEEAVRLFCEWLKNGGPSSGAPPPS

SEQ ID NO: 102 - protein, 39 aa, Artificial Sequence
  Description of Artificial Sequence Synthetic polypeptide
HGEGTFTSDLSKQMEEEAVRLFICWLKNGGPSSGAPPPS

SEQ ID NO: 103 - protein, 39 aa, Artificial Sequence
  Description of Artificial Sequence Synthetic polypeptide
HGEGTFTSDLSKQMEEEAVRLFIECLKNGGPSSGAPPPS

SEQ ID NO: 104 - protein, 39 aa, Artificial Sequence
  Description of Artificial Sequence Synthetic polypeptide
HGEGTFTSDLSKQMEEEAVRLFIEWCKNGGPSSGAPPPS

SEQ ID NO: 105 - protein, 39 aa, Artificial Sequence
  Description of Artificial Sequence Synthetic polypeptide
HGEGTFTSDLSKQMEEEAVRLFIEWLCNGGPSSGAPPPS

SEQ ID NO: 106 - protein, 39 aa, Artificial Sequence
  Description of Artificial Sequence Synthetic polypeptide
HGEGTFTSDLSKQMEEEAVRLFIEWLKCGGPSSGAPPPS

SEQ ID NO: 107 - protein, 39 aa, Artificial Sequence
  Description of Artificial Sequence Synthetic polypeptide
HGEGTFTSDLSKQMEEEAVRLFIEWLKNCGPSSGAPPPS

SEQ ID NO: 108 - protein, 39 aa, Artificial Sequence
  Description of Artificial Sequence Synthetic polypeptide
HGEGTFTSDLSKQMEEEAVRLFIEWLKNGCPSSGAPPPS

SEQ ID NO: 109 - protein, 39 aa, Artificial Sequence
  Description of Artificial Sequence Synthetic polypeptide
HGEGTFTSDLSKQMEEEAVRLFIEWLKNGGCSSGAPPPS

SEQ ID NO: 110 - protein, 39 aa, Artificial Sequence
  Description of Artificial Sequence Synthetic polypeptide
HGEGTFTSDLSKQMEEEAVRLFIEWLKNGGPCSGAPPPS

SEQ ID NO: 111 - protein, 39 aa, Artificial Sequence
  Description of Artificial Sequence Synthetic polypeptide
HGEGTFTSDLSKQMEEEAVRLFIEWLKNGGPSCGAPPPS

SEQ ID NO: 112 - protein, 39 aa, Artificial Sequence
  Description of Artificial Sequence Synthetic polypeptide
HGEGTFTSDLSKQMEEEAVRLFIEWLKNGGPSSCAPPPS

SEQ ID NO: 113 - protein, 39 aa, Artificial Sequence
  Description of Artificial Sequence Synthetic polypeptide
HGEGTFTSDLSKQMEEEAVRLFIEWLKNGGPSSGCPPPS

SEQ ID NO: 114 - protein, 39 aa, Artificial Sequence
  Description of Artificial Sequence Synthetic polypeptide
HGEGTFTSDLSKQMEEEAVRLFIEWLKNGGPSSGACPPS

SEQ ID NO: 115 - protein, 39 aa, Artificial Sequence
  Description of Artificial Sequence Synthetic polypeptide
HGEGTFTSDLSKQMEEEAVRLFIEWLKNGGPSSGAPCPS

SEQ ID NO: 116 - protein, 39 aa, Artificial Sequence
  Description of Artificial Sequence Synthetic polypeptide
HGEGTFTSDLSKQMEEEAVRLFIEWLKNGGPSSGAPPCS

SEQ ID NO: 117 - protein, 39 aa, Artificial Sequence
  Description of Artificial Sequence Synthetic polypeptide
HGEGTFTSDLSKQMEEEAVRLFIEWLKNGGPSSGAPPPC

SEQ ID NO: 118 - protein, 40 aa, Artificial Sequence
  Description of Artificial Sequence Synthetic polypeptide
CHGEGTFTSDLSKQMEEEAVRLFIEWLKNGGPSSGAPPPS

SEQ ID NO: 119 - protein, 40 aa, Artificial Sequence
  Description of Artificial Sequence Synthetic polypeptide
HGEGTFTSDLSKQMEEEAVRLFIEWLKNGGPSSGAPPPSC

SEQ ID NO: 120 - protein, 39 aa, Artificial Sequence; identical to SEQ ID NO: 79
  Description of Artificial Sequence Synthetic polypeptide

SEQ ID NO: 121 - protein, 39 aa, Artificial Sequence; identical to SEQ ID NO: 80
  Description of Artificial Sequence Synthetic polypeptide

SEQ ID NO: 122 - protein, 39 aa, Artificial Sequence; identical to SEQ ID NO: 81
  Description of Artificial Sequence Synthetic polypeptide

SEQ ID NO: 123 - protein, 39 aa, Artificial Sequence; identical to SEQ ID NO: 82
  Description of Artificial Sequence Synthetic polypeptide

SEQ ID NO: 124 - protein, 39 aa, Artificial Sequence; identical to SEQ ID NO: 83
  Description of Artificial Sequence Synthetic polypeptide

SEQ ID NO: 125 - protein, 39 aa, Artificial Sequence; identical to SEQ ID NO: 84
  Description of Artificial Sequence Synthetic polypeptide

SEQ ID NO: 126 - protein, 39 aa, Artificial Sequence; identical to SEQ ID NO: 85
  Description of Artificial Sequence Synthetic polypeptide

SEQ ID NO: 127 - protein, 39 aa, Artificial Sequence; identical to SEQ ID NO: 86
  Description of Artificial Sequence Synthetic polypeptide

SEQ ID NO: 128 - protein, 39 aa, Artificial Sequence; identical to SEQ ID NO: 87
  Description of Artificial Sequence Synthetic polypeptide

SEQ ID NO: 129 - protein, 39 aa, Artificial Sequence; identical to SEQ ID NO: 88
  Description of Artificial Sequence Synthetic polypeptide

SEQ ID NO: 130 - protein, 39 aa, Artificial Sequence; identical to SEQ ID NO: 89
  Description of Artificial Sequence Synthetic polypeptide

SEQ ID NO: 131 - protein, 39 aa, Artificial Sequence; identical to SEQ ID NO: 90
  Description of Artificial Sequence Synthetic polypeptide

SEQ ID NO: 132 - protein, 39 aa, Artificial Sequence; identical to SEQ ID NO: 91
  Description of Artificial Sequence Synthetic polypeptide

SEQ ID NO: 133 - protein, 39 aa, Artificial Sequence; identical to SEQ ID NO: 92
  Description of Artificial Sequence Synthetic polypeptide

SEQ ID NO: 134 - protein, 39 aa, Artificial Sequence; identical to SEQ ID NO: 93
  Description of Artificial Sequence Synthetic polypeptide

SEQ ID NO: 135 - protein, 39 aa, Artificial Sequence; identical to SEQ ID NO: 94
  Description of Artificial Sequence Synthetic polypeptide

SEQ ID NO: 136 - protein, 39 aa, Artificial Sequence; identical to SEQ ID NO: 95
  Description of Artificial Sequence Synthetic polypeptide

SEQ ID NO: 137 - protein, 39 aa, Artificial Sequence; identical to SEQ ID NO: 96
  Description of Artificial Sequence Synthetic polypeptide

SEQ ID NO: 138 - protein, 39 aa, Artificial Sequence; identical to SEQ ID NO: 97
  Description of Artificial Sequence Synthetic polypeptide

SEQ ID NO: 139 - protein, 39 aa, Artificial Sequence; identical to SEQ ID NO: 98
  Description of Artificial Sequence Synthetic polypeptide

SEQ ID NO: 140 - protein, 39 aa, Artificial Sequence; identical to SEQ ID NO: 99
  Description of Artificial Sequence Synthetic polypeptide

SEQ ID NO: 141 - protein, 39 aa, Artificial Sequence; identical to SEQ ID NO: 100
  Description of Artificial Sequence Synthetic polypeptide

SEQ ID NO: 142 - protein, 39 aa, Artificial Sequence; identical to SEQ ID NO: 101
  Description of Artificial Sequence Synthetic polypeptide

SEQ ID NO: 143 - protein, 39 aa, Artificial Sequence; identical to SEQ ID NO: 102
  Description of Artificial Sequence Synthetic polypeptide

SEQ ID NO: 144 - protein, 39 aa, Artificial Sequence; identical to SEQ ID NO: 103
  Description of Artificial Sequence Synthetic polypeptide

SEQ ID NO: 145 - protein, 39 aa, Artificial Sequence; identical to SEQ ID NO: 104
  Description of Artificial Sequence Synthetic polypeptide

SEQ ID NO: 146 - protein, 39 aa, Artificial Sequence; identical to SEQ ID NO: 105
  Description of Artificial Sequence Synthetic polypeptide

SEQ ID NO: 147 - protein, 39 aa, Artificial Sequence; identical to SEQ ID NO: 106
  Description of Artificial Sequence Synthetic polypeptide

SEQ ID NO: 148 - protein, 39 aa, Artificial Sequence; identical to SEQ ID NO: 107
  Description of Artificial Sequence Synthetic polypeptide

SEQ ID NO: 149 - protein, 39 aa, Artificial Sequence; identical to SEQ ID NO: 108
  Description of Artificial Sequence Synthetic polypeptide

SEQ ID NO: 150 - protein, 39 aa, Artificial Sequence; identical to SEQ ID NO: 109
  Description of Artificial Sequence Synthetic polypeptide

SEQ ID NO: 151 - protein, 39 aa, Artificial Sequence; identical to SEQ ID NO: 110
  Description of Artificial Sequence Synthetic polypeptide

SEQ ID NO: 152 - protein, 39 aa, Artificial Sequence; identical to SEQ ID NO: 111
  Description of Artificial Sequence Synthetic polypeptide

SEQ ID NO: 153 - protein, 39 aa, Artificial Sequence; identical to SEQ ID NO: 112
  Description of Artificial Sequence Synthetic polypeptide

SEQ ID NO: 154 - protein, 39 aa, Artificial Sequence; identical to SEQ ID NO: 113
  Description of Artificial Sequence Synthetic polypeptide

SEQ ID NO: 155 - protein, 39 aa, Artificial Sequence; identical to SEQ ID NO: 114
  Description of Artificial Sequence Synthetic polypeptide

SEQ ID NO: 156 - protein, 39 aa, Artificial Sequence; identical to SEQ ID NO: 115
  Description of Artificial Sequence Synthetic polypeptide

SEQ ID NO: 157 - protein, 39 aa, Artificial Sequence; identical to SEQ ID NO: 116
  Description of Artificial Sequence Synthetic polypeptide

SEQ ID NO: 158 - protein, 39 aa, Artificial Sequence; identical to SEQ ID NO: 117
  Description of Artificial Sequence Synthetic polypeptide

SEQ ID NO: 159 - protein, 40 aa, Artificial Sequence; identical to SEQ ID NO: 118
  Description of Artificial Sequence Synthetic polypeptide

SEQ ID NO: 160 - protein, 40 aa, Artificial Sequence; identical to SEQ ID NO: 119
  Description of Artificial Sequence Synthetic polypeptide

SEQ ID NO: 161 - protein, 38 aa, Artificial Sequence
  Description of Artificial Sequence Synthetic polypeptide
XXXXXXXXXXXXXXXXXXXXXXXXXXXXXXXXXXXXXX

SEQ ID NO: 162 - protein, 41 aa, Artificial Sequence
  Description of Artificial Sequence Synthetic polypeptide
XXXXXXXXXXXXXXXXXXXXXXXXXXXXXXXXXXXXXXXXX

SEQ ID NO: 163 - protein, 10 aa, Artificial Sequence
  Description of Artificial Sequence Synthetic peptide
NQQEKNEQEL

SEQ ID NO: 164 - protein, 5 aa, Artificial Sequence
  Description of Artificial Sequence Synthetic peptide
IEESQ

SEQ ID NO: 165 - protein, 5 aa, Artificial Sequence
  Description of Artificial Sequence Synthetic peptide
LELDK

SEQ ID NO: 166 - protein, 4 aa, Artificial Sequence
  Description of Artificial Sequence Synthetic peptide
IHSL

SEQ ID NO: 167 - protein, 4 aa, Artificial Sequence
  Description of Artificial Sequence Synthetic peptide
WASL